(19) United States
(12) Patent Application Publication
(10) Pub. No.: US 2026/0248614 A1
(43) Pub. Date: Aug. 27, 2026

(54) SYSTEMS AND METHODS FOR TREATING A NATIVE VALVE

(71) Applicant: Edwards Lifesciences Corporation, Irvine, CA (US)

(72) Inventors: Nikolai Brent Poulsen, Tustin, CA (US); Mark Chau, Laguna Hills, CA (US); Sam Shafigh, Mission Viejo, CA (US); Travis Zenyo Oba, Yorba Linda, CA (US); Andrew Charles May, Irvine, CA (US); Alana Tyler Stein, Newport Beach, CA (US); Nicolas Schleiger, Huntington Beach, CA (US); Paul Kaye, Modiin (IL); Bhumica A. Amin, Modesto, CA (US); David M. Taylor, Lake Forest, CA (US); Bar Eytan-Vaisman, Gedera (IL)

(21) Appl. No.: 19/650,166

(22) Filed: Apr. 16, 2026

Related U.S. Application Data
(63) Continuation of application No. PCT/US2024/050783, filed on Oct. 10, 2024.
(60) Provisional application No. 63/612,293, filed on Dec. 19, 2023.
(60) Provisional application No. 63/591,745, filed on Oct. 19, 2023.

(51) Int. Cl. A61F 2/24; A61B 18/14; A61B 18/00

(52) U.S. Cl. CPC A61F 2/2463; A61B 18/1492; A61F 2/246; A61F 2/2466; A61B 2018/00369; A61F 2250/0059

(57) ABSTRACT

A device configured to be positioned within a native heart valve to repair the valve. The device includes a leaflet pinching or capturing mechanism to capture a first leaflet and second leaflet of the native valve and then attach the first leaflet to the second leaflet. The device includes a first surface and a second surface, wherein the first surface and the second surface are movable between an open condition and a closed condition to pinch a first leaflet and a second leaflet of the native valve therebetween. Once the first and second leaflets are captured, portions of the leaflets are fused together, adhered together, or fused and adhered together. In some implementations, a system is configured to access a papillary muscle and ablate tissue of the papillary muscle to disable a conductive pathway to the papillary muscle.

### RELATED APPLICATIONS

The present application is a continuation of Patent Cooperation Treaty Application number PCT/US2024/050783, filed on October 10, 2024, which claims the benefit of US Provisional Patent Application No. 63/612,293, filed on December 19, 2023, and US Provisional Patent Application No. 63/591,745, filed October 19, 2023, which are all incorporated herein by reference in their entireties.

### BACKGROUND

The native heart valves (i.e., the aortic, pulmonary, tricuspid, and mitral valves) serve critical functions in assuring the forward flow of an adequate supply of blood through the cardiovascular system. These heart valves may be damaged, and thus rendered less effective, for example, by congenital malformations, inflammatory processes, infectious conditions, disease, etc. Such damage to the valves may result in serious cardiovascular compromise or death. Damaged valves can be surgically repaired or replaced during open heart surgery. However, open heart surgeries are highly invasive, and complications may occur. Transvascular techniques can be used to introduce and deploy/implant devices to treat a heart in a manner that is much less invasive than open heart surgery. As one example, a transvascular technique useable for accessing the native mitral and aortic valves is the trans-septal technique. The trans-septal technique comprises advancing a catheter into the right atrium (e.g., inserting a catheter into the right femoral vein, up the inferior vena cava and into the right atrium). The septum is then punctured, and the catheter passed into the left atrium. A similar transvascular technique can be used to deploy/implant a device within the tricuspid valve that begins similarly to the trans-septal technique but stops short of puncturing the septum and instead turns the delivery catheter toward the tricuspid valve in the right atrium.

A healthy heart has a generally conical shape that tapers to a lower apex. The heart is four-chambered and comprises the left atrium, right atrium, left ventricle, and right ventricle. The left and right sides of the heart are separated by a wall generally referred to as the septum. The native mitral valve of the human heart connects the left atrium to the left ventricle. The mitral valve has a very different anatomy than other native heart valves. The mitral valve includes an annulus portion, which is an annular portion of the native valve tissue surrounding the mitral valve orifice, and a pair of cusps, or leaflets, extending downward from the annulus into the left ventricle. The mitral valve annulus may form a “D”-shaped, oval, or otherwise out-of-round cross-sectional shape having major and minor axes. The anterior leaflet may be larger than the posterior leaflet, forming a generally “C”-shaped boundary between the abutting sides of the leaflets when they are closed together.

When operating properly, the anterior leaflet and the posterior leaflet function together as a one-way valve to allow blood to flow only from the left atrium to the left ventricle. The left atrium receives oxygenated blood from the pulmonary veins. When the muscles of the left atrium contract and the left ventricle dilates (also referred to as “ventricular diastole” or “diastole”), the oxygenated blood that is collected in the left atrium flows into the left ventricle. When the muscles of the left atrium relax and the muscles of the left ventricle contract (also referred to as “ventricular systole” or “systole”), the increased blood pressure in the left ventricle urges the sides of the two leaflets together, thereby closing the one-way mitral valve so that blood cannot flow back to the left atrium and is instead expelled out of the left ventricle through the aortic valve. To prevent or inhibit the two leaflets from prolapsing under pressure and folding back through the mitral annulus toward the left atrium, a plurality of fibrous cords called chordae tendineae tether the leaflets to papillary muscles in the left ventricle.

Valvular regurgitation involves the valve improperly allowing some blood to flow in the wrong direction through the valve. For example, mitral regurgitation occurs when the native mitral valve fails to close properly and blood flows into the left atrium from the left ventricle during the systolic phase of heart contraction. Mitral regurgitation is one of the most common forms of valvular heart disease. Mitral regurgitation may have many different causes, such as leaflet prolapse, dysfunctional papillary muscles, stretching of the mitral valve annulus resulting from dilation of the left ventricle, more than one of these, etc. Mitral regurgitation at a central portion of the leaflets can be referred to as central jet mitral regurgitation and mitral regurgitation nearer to one commissure (i.e., location where the leaflets meet) of the leaflets can be referred to as eccentric jet mitral regurgitation. Central jet regurgitation occurs when the edges of the leaflets do not meet in the middle and thus the valve does not close, and regurgitation is present. Tricuspid regurgitation may be similar, but on the right side of the heart.

This summary is meant to provide some examples and is not intended to be limiting of the scope of the disclosed subject matter in any way. For example, any feature included in an example of this summary is not required by the claims, unless the claims explicitly recite the feature. Also, the features, components, steps, concepts, etc. described in examples in this summary and elsewhere in this disclosure can be combined in a variety of ways. Various features and steps as described elsewhere in this disclosure can be included in the examples summarized here.

Devices for repairing and/or treating a native valve of a patient are disclosed. The devices can be valve repair devices, implantable devices, valve treatment devices, implants, etc. While the devices may be described and/or depicted as implantable devices in some examples herein, similar configurations can be used on other devices, e.g., valve repair devices, treatment devices, etc., that are not necessarily implanted and may be removed after treatment.

In some implementations, a device for repairing a native valve is configured to be positioned within the native heart valve. In some implementations, the device includes a leaflet pinching or capturing mechanism to capture a first leaflet and second leaflet of the native valve. In some implementations, the device is configured to attach (e.g., fuse, adhere, connect, etc.) the first leaflet and the second leaflet together.

In some implementations, a device for repairing a native valve includes a first surface (e.g., a first surface of a first arm of the device, etc.) and a second surface (e.g., a second surface of a second arm of the device, etc.). The first surface and the second surface are movable between an open condition and a closed condition to pinch a first leaflet and a second leaflet of the native valve therebetween.

In some implementations, the first surface is a surface of a first arm of the device and the second surface is a surface of a second arm of the device. In some implementations, the first surface and/or first arm and the second surface and/or second arm are movable between an open condition and a closed condition to pinch a first leaflet and a second leaflet of the native valve therebetween.

In some implementations, the device is configured to attach (e.g., fuse, adhere, connect, etc.) the first leaflet to the second leaflet. In some implementations, the device is configured to directly attach the first leaflet to the second leaflet. In some implementations, the device is configured to indirectly attach the first leaflet to the second leaflet (e.g., with each leaflet attached to an intermediate component, such as a coaptation element).

In some implementations, the device is configured to attach the first leaflet to the second leaflet by fusing the first leaflet to the second leaflet. In some implementations, the device includes a radio frequency generator operatively coupled to one or more of the first surface and the second surface.

In some implementations, the first surface is a first contact surface on a first arm of the device and the second surface is a second contact surface on a second arm of the device. In some implementations, the first contact surface and the second contact surface are configured to output energy to the first leaflet and the second leaflet, respectively, to fuse the first leaflet to the second leaflet. In some implementations, the first contact surface and the second contact surface are configured to fuse the first leaflet to the second leaflet at two or more spaced apart locations.

In some implementations, one or more of the first surface, the first arm, the second surface, and/or the second arm has an inner face that is U-shaped.

In some implementations, the device includes a coaptation element. In some implementations, the first surface and/or first arm is configured to capture the first leaflet against the coaptation element. In some implementations, the second surface and/or second arm is configured to capture the second leaflet against the coaptation element. I

In some implementations, the second surface and/or second arm is movable to capture the second leaflet independent of the first surface and/or second arm.

In some implementations, the device is configured to attach the first leaflet to the second leaflet indirectly via a coaptation element.

In some implementations, the coaptation element is movable from between the first leaflet and the second leaflet prior to attaching the first leaflet to the second leaflet. In some implementations, the coaptation element is movable from between the first leaflet and the second leaflet after the first leaflet is attached to the second leaflet.

In some implementations, the first surface is configured to fuse the first leaflet to the second leaflet at a first location lateral to the coaptation element and at a second location lateral to the coaptation element and opposite the first location.

In some implementations, the first surface is configured to capture the first leaflet against a first portion of the coaptation element and against a second portion of the coaptation element, and wherein the first surface is configured to fuse the first leaflet to the second leaflet at a location between the first portion and the second portion.

In some implementations, the first surface is configured to fuse the first leaflet to the coaptation element. In some implementations, the second surface is configured to fuse the second leaflet to the coaptation element. In some implementations, the first surface is configured to fuse the first leaflet to the coaptation element independent of the second surface.

In some implementations, the coaptation element has an exterior surface configured to be fused to the first leaflet, wherein the exterior surface is cylindrical. In some implementations, the coaptation element has an exterior surface configured to be fused to the first leaflet, wherein the exterior surface is conical. In some implementations, the exterior surface comprises collagen.

In some implementations, the device includes a third surface (e.g., a surface of a third arm, etc.) and a fourth surface (e.g., a surface of a fourth arm, etc.). In some implementations, the third surface and the fourth surface are movable between an open condition and a closed condition to pinch the first leaflet and the second leaflet of the native valve therebetween at a second location spaced apart from the first location at which the first surface and the second surface pinch the first leaflet and the second leaflet together.

In some implementations, the first surface and/or first arm has a first inner face and the second surface and/or second arm has a second inner face. In some implementations, the first inner face and the second inner face have complementary textured surface configurations. In some implementations, the textured surface configuration comprises an undulating surface. In some implementations, the textured surface configuration comprises a plurality of projections.

In some implementations, the device is configured to attach the first leaflet to the second leaflet with an adhesive. In some implementations, the device includes a coaptation element. In some implementations, the first surface and/or first arm is configured to capture the first leaflet against the coaptation element.

In some implementations, the adhesive is embedded in the coaptation element. In some implementations, the coaptation element comprises a spongey material infused with the adhesive. In some implementations, the coaptation element and/or spongey material includes additional attachment-enhancing features, such as barbs, hooks, folds, ridges, extensions, loops, etc.

In some implementations, the device includes an adhesive delivery conduit configured to deliver the adhesive between the first leaflet and the second leaflet. In some implementations, the adhesive delivery conduit includes a static mixer configured to mix the adhesive prior to the adhesive being delivered between the first leaflet and the second leaflet.

In some implementations, at least one component of the adhesive is encapsulated by an encapsulating structure. In some implementations, the encapsulating structure is configured to de-encapsulate when the adhesive is positioned between the first leaflet and the second leaflet, and the first surface and the second surface pinch the first leaflet and second leaflet therebetween. In some implementations, the encapsulating structure is dissolvable. In some implementations, the adhesive is a two-component adhesive.

In some implementations, the adhesive is embedded in a substate attached to an exterior surface of the coaptation element. In some implementations, the adhesive is disposed onto a substrate attached to an exterior surface of the coaptation element. In some implementations, the adhesive is disposed on an exterior surface of the coaptation element. In some implementations, the coaptation element and/or substrate includes additional attachment-enhancing features, such as barbs, hooks, folds, ridges, extensions, loops, etc.

In some implementations, the exterior surface of the coaptation element includes a textured surface configuration. In some implementations, the textured surface configuration comprises a plurality of recesses and projections.

In some implementations, a system for repairing a native valve includes a delivery system for delivering the device to the native valve. In some implementations, the delivery system includes a catheter, and the device is operatively coupled to a distal end of the catheter.

In some implementations, a method for repairing a native valve includes pinching a first leaflet of the native valve and a second leaflet of the native valve together and attaching the first leaflet to the second leaflet.

In some implementations, attaching the first leaflet to the second leaflet further comprises fusing the first leaflet to the second leaflet. In some implementations, fusing the first leaflet to the second leaflet with radio frequency energy. In some implementations, fusing the first leaflet to the second leaflet further includes fusing the first leaflet to the second leaflet at two or more spaced apart locations.

In some implementations, the method includes capturing the first leaflet against a coaptation element. In some implementations, the method includes capturing the second leaflet against the coaptation element. In some implementations, the second leaflet is captured independent of the first leaflet.

In some implementations, the method includes removing the coaptation element from between the first leaflet and the second leaflet prior to attaching the first leaflet to the second leaflet. In some implementations, the method includes removing the coaptation element from between the first leaflet and the second leaflet after attaching the first leaflet to the second leaflet.

In some implementations, fusing the first leaflet to the second leaflet includes fusing the first leaflet to the second leaflet at a first location lateral to the coaptation element and fusing the first leaflet to the second leaflet at a second location lateral to the coaptation element and opposite the first location.

In some implementations, capturing the first leaflet against the coaptation element includes pinching the first leaflet against the coaptation element at a first location and at a second location spaced apart from the first location.

In some implementations, fusing the first leaflet to the second leaflet further includes fusing the first leaflet to the second leaflet at a third location between the first location and the second location.

In some implementations, attaching the first leaflet to the second leaflet includes fusing the first leaflet and the second leaflet to an exterior surface of the coaptation element.

In some implementations, pinching the first leaflet of the native valve and the second leaflet of the native valve together includes indenting the first leaflet and the second leaflet.

In some implementations, the first leaflet of the native valve and the second leaflet of the native valve together includes creating an undulating pattern in the first leaflet and the second leaflet.

In some implementations, attaching the first leaflet to the second leaflet includes attaching the first leaflet to the second leaflet with an adhesive.

In some implementations, the method includes capturing the first leaflet against a coaptation element.

In some implementations, pinching the first leaflet of the native valve and the second leaflet of the native valve together includes pinching the coaptation element between the first leaflet and the second leaflet.

In some implementations, the adhesive is embedded in the coaptation element and pinching the coaptation element between the first leaflet and the second leaflet releases the adhesive.

In some implementations, the coaptation element includes additional attachment-enhancing features, such as barbs, hooks, folds, ridges, extensions, loops, etc.

In some implementations, adhesive is delivered between the first leaflet and the second leaflet via an adhesive delivery conduit.

In some implementations, the method includes mixing the adhesive within the adhesive delivery conduit. In some implementations, the method includes mixing two or more components of the adhesive together within the adhesive delivery conduit.

In some implementations, the adhesive is encapsulated by an encapsulating structure. In some implementations, the method includes positioning the adhesive encapsulated in the encapsulating structure between the first leaflet and the second leaflet and pinching the first leaflet and the second leaflet together to de-encapsulate adhesive. In some implementations, de-encapsulating the adhesive includes bursting the encapsulating structure.

In some implementations, the method includes disposing the adhesive on a substrate attached to an exterior surface of the coaptation element. In some implementations, the method includes disposing the adhesive onto an exterior surface of the coaptation element.

In some implementations, the exterior surface of the coaptation element includes a plurality of recesses and projections, and wherein the adhesive is received in the plurality of recesses.

In some implementations, the coaptation element and/or substrate includes additional attachment-enhancing features, such as barbs, hooks, folds, ridges, extensions, loops, etc.

In some implementations, a method for repairing a native valve includes navigating a distal end of a catheter to a location inside of a heart proximate tissue of the heart (e.g., proximate a papillary muscle, proximate a heart wall, etc.), extending an ablation device from the distal end of the catheter to the tissue of the heart (e.g., to a papillary muscle, to a heart wall, etc.), and ablating the tissue with the ablation device to disable a conductive pathway to the tissue (e.g., to a papillary muscle, to a heart wall, etc.).

In some implementations, the method includes inserting the catheter through the mitral valve or through the tricuspid valve.

In some implementations, the method includes detecting electrical signals transmitted through the conductive pathway. In some implementations, an electrode associated with the catheter is configured to detect the electrical signals.

In some implementations, the method includes selecting the tissue (e.g., selecting a papillary muscle, etc.) based on detected electrical signals.

In some implementations, the method includes selecting the tissue (e.g., selecting a papillary muscle, etc.) based on a geometry of a ventricle of the heart and/or a leaflet of the native valve.

In some implementations, the ablation device is used to ablate tissue on only one papillary muscle. In some implementations, the ablation device is used to ablate tissue on each papillary muscle.

In some implementations, the ablation device is used to ablate tissue repeatedly on a single papillary muscle.

In some implementations, the ablation device is a radiofrequency ablation device. In some implementations, the ablation device is used to ablate tissue is a cryoablation device.

Any of the above method(s) and any methods of using the systems, assemblies, apparatuses, devices, etc. herein can be performed on a living subject (e.g., human or other animal) or on a simulation (e.g., a cadaver, cadaver heart, imaginary person, simulator, etc.). With a simulation, the body parts can optionally be referred to as “simulated” (e.g., simulated heart, simulated tissue, etc.) and can optionally comprise computerized and/or physical representations.

Any of the above systems, assemblies, devices, apparatuses, components, etc. can be sterilized (e.g., with heat, radiation, ethylene oxide, hydrogen peroxide, etc.) to ensure they are safe for use with patients, and the methods herein can comprise (or additional methods comprise or consist of) sterilization of one or more systems, devices, apparatuses, components, etc. herein (e.g., with heat, radiation, ethylene oxide, hydrogen peroxide, etc.).

A further understanding of the nature and advantages of the present invention are set forth in the following description and claims, particularly when considered in conjunction with the accompanying drawings in which like parts bear like reference numerals.

### BRIEF DESCRIPTION OF THE DRAWINGS

To further clarify various aspects of examples in the present disclosure, a more particular description of certain examples and implementations will be made by reference to various aspects of the appended drawings. These drawings depict only example implementations of the present disclosure and are therefore not to be considered limiting of the scope of the disclosure. Moreover, while the figures can be drawn to scale for some examples, the figures are not necessarily drawn to scale for all examples. Examples and other features and advantages of the present disclosure will be described and explained with additional specificity and detail through the use of the accompanying drawings in which:

FIG. 1 illustrates a cutaway view of the human heart in a diastolic phase;

FIG. 2 illustrates a cutaway view of the human heart in a systolic phase;

FIG. 3 illustrates a cutaway view of the human heart in a systolic phase showing valve regurgitation;

FIG. 4 is the cutaway view of FIG. 3 annotated to illustrate a natural shape of mitral valve leaflets in the systolic phase;

FIG. 5 illustrates a healthy mitral valve with the leaflets closed as viewed from an atrial side of the mitral valve;

FIG. 6 illustrates a dysfunctional mitral valve with a visible gap between the leaflets as viewed from an atrial side of the mitral valve;

FIG. 7 illustrates a tricuspid valve viewed from an atrial side of the tricuspid valve;

FIGS. 8–14 show an example device or implant, in various stages of deployment;

FIG. 15 shows an example device that is similar to the device illustrated by FIGS. 8–14, but where the paddles are independently controllable;

FIGS. 16–21 show the example device of FIGS. 8–14 being delivered and deployed within a native valve;

FIGS. 21A-21C show various implementations of an example device or implant similar to the device or implant of FIGS. 8–14;

FIG. 22 shows a perspective view of an example device in a closed position;

FIG. 23 shows a perspective view of an example device in a closed position;

FIG. 24 illustrates an example device with paddles in an open position;

FIG. 25A illustrates an example device with paddles in a closed position;

FIG. 25B illustrates a top view of an example device;

FIG. 26 illustrates a perspective view of an example device having paddles of adjustable widths;

FIG. 27 is a cross-section of the example device of FIG. 26 in which the device is bisected;

FIG. 28 is another cross-section of the example device of FIG. 26 in which the device is bisected along a plane perpendicular to the plane illustrated in FIG. 28;

FIG. 29 is a schematic illustration of an example catheter assembly coupled to an example device in which an actuation element is coupled to a paddle actuation control and to a driver head of the device;

FIG. 30 is an illustration of the assembly of FIG. 29 with the example device rotated 90 degrees to show the paddle width adjustment element coupled to an inner end of the connector of the device and coupled to a paddle width control;

FIG. 31-33 illustrate an example leaflet attachment device being delivered and deployed within a native valve;

FIG. 34 illustrates an end view of the device of FIGS. 31-33;

FIG. 34A illustrates an end view of a device that is similar to the device of FIGS. 31-36, but the device has only a single pair of sealing or projecting portions;

FIG. 35 illustrates a sectional view of the device of FIG. 34 through the 35-35 line;

FIG. 36 illustrates a sectional view of the device of FIG. 34 through the 36-36 line;

FIG. 37 illustrates an end view of an example leaflet attachment device;

FIG. 38 illustrates a sectional view of the device of FIG. 37 through the 38-38 line;

FIG. 39 illustrates the leaflet attachment device of FIGS. 31-33 being delivered via the interior vena cava;

FIG. 40 illustrates the leaflet attachment device of FIGS. 31-33 being delivered via the ascending aorta;

FIG. 40A illustrates the leaflet attachment device of FIGS. 31-33 being delivered transeptally into the left atrium;

FIGS. 41-44 illustrate an example leaflet attachment device being delivered and deployed within a native valve;

FIG. 45 illustrates two leaflets sealed together at a pair of seals applied by a leaflet attachment device;

FIG. 45A illustrates two leaflets sealed together at a single seal applied by a leaflet attachment device;

FIGS. 46-47 illustrate an example leaflet attachment device being delivered and deployed within a native valve;

FIG. 48 illustrates a pair of fused tissue areas between leaflets made by the leaflet attachment device illustrated by FIGS. 46-47;

FIG. 48A illustrates single fused tissue area between leaflets made by a modified version of leaflet attachment device illustrated by FIGS. 46-47 that fuses tissue together in only one area;

FIGS. 49-50 illustrate an example leaflet attachment device being delivered and deployed within a native valve;

FIG. 51 illustrates a spacer attached to leaflets of a native valve;

FIGS. 52-54 illustrate an example leaflet attachment device engaging leaflets of a native valve;

FIG. 55 illustrates an example dimple for a leaflet attachment device;

FIG. 56 is a cross-section of the dimple of FIG. 55;

FIGS. 57-58 illustrate a leaflet of a native heart valve after being engaged by a leaflet attachment device having a plurality of the dimples of FIG. 55;

FIG. 59 illustrates an example leaflet attachment device being delivered via the ascending aorta and utilizing RF energy to attach leaflets of a native heart valve;

FIG. 60 illustrates leaflets of a native heart valve attached by an adhesive;

FIGS. 61-62 illustrate an example leaflet attachment device utilizing an adhesive;

FIG. 63 illustrates an example leaflet attachment device utilizing an adhesive;

FIGS. 64-65 illustrate an example leaflet attachment device utilizing an adhesive;

FIGS. 66-67 illustrate an example leaflet attachment device utilizing an adhesive;

FIGS. 68-70 illustrate an example leaflet attachment device utilizing an adhesive;

FIGS. 71-73 illustrates an example valve repair device used in conjunction with an adhesive;

FIG. 74 illustrates an example valve repair device;

FIG. 75 illustrates a cutaway view of the human heart in a systolic phase showing mitral valve regurgitation caused by dilation of the left ventricle;

FIG. 76 illustrates an example process for causing relaxation of heart tissue; and

FIG. 77 illustrates the cutaway view of the human heart of FIG. 75 with papillary muscles relaxed to permit coaptation of the mitral valve leaflets.

### DETAILED DESCRIPTION

The following description refers to the accompanying drawings, which illustrate example implementations of the present disclosure. Other implementations having different structures and operation do not depart from the scope of the present disclosure.

Some implementations of the present disclosure are directed to systems, devices, methods, etc. for repairing a defective heart valve. For example, some implementations of devices, valve treatment devices, valve repair devices, implantable devices, implants, and systems (including systems for delivery thereof) are disclosed herein, and any combination of these options can be made unless specifically excluded. In other words, individual components of the disclosed devices and systems can be combined unless mutually exclusive or otherwise physically impossible.

The techniques, methods, operations, steps, etc. described or suggested herein or in the references incorporated herein, and any methods of using the systems, assemblies, apparatuses, devices, etc. herein, can be performed on a living subject (e.g., human, other animal, etc.) or on a simulation (e.g., a cadaver, cadaver heart, simulator, imaginary person, etc.). When performed on a simulation, the body parts, e.g., heart, tissue, valve, etc., can be assumed to be simulated or can optionally be referred to as “simulated” (e.g., simulated heart, simulated tissue, simulated valve, etc.) and can optionally comprise computerized and/or physical representations of body parts, tissue, etc. The term “simulation” covers use on a cadaver, computer simulator, imaginary person (e.g., if they are just demonstrating in the air on an imaginary heart), etc.

As described herein, when one or more components are described as being connected, joined, affixed, coupled, attached, or otherwise interconnected, such interconnection can be direct as between the components or can be indirect such as through the use of one or more intermediary components. Also as described herein, reference to a "member," “component,” or “portion” shall not be limited to a single structural member, component, or element but can include an assembly of components, members, or elements. Also as described herein, the terms “substantially” and “about” are defined as at least close to (and includes) a given value or state (preferably within 10% of, more preferably within 1% of, and most preferably within 0.1% of). The terms “clasp” and “clasp arm” are often used herein with respect to specific examples, but the terms “gripping member” and/or “gripper arm” can be used in place of and function in the same or similar ways, even if not configured in the same way as a typical clasp.

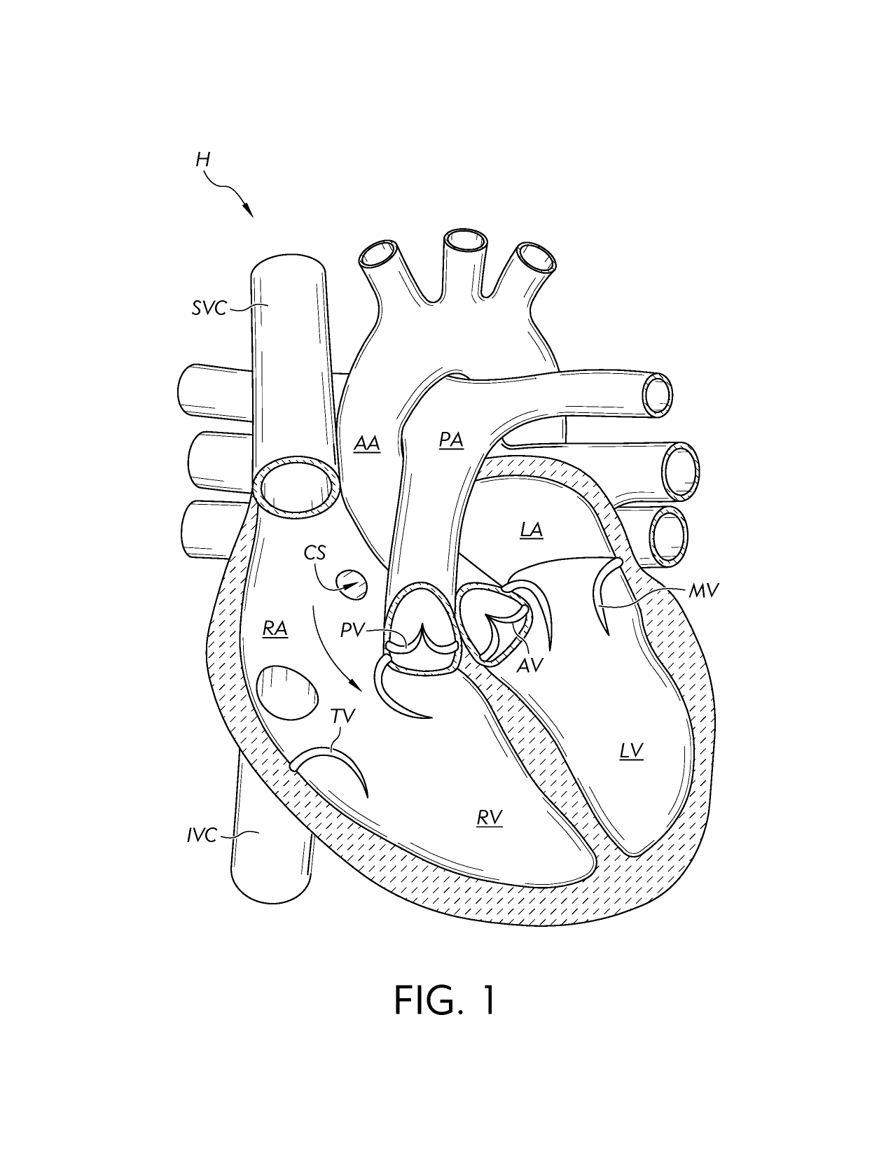

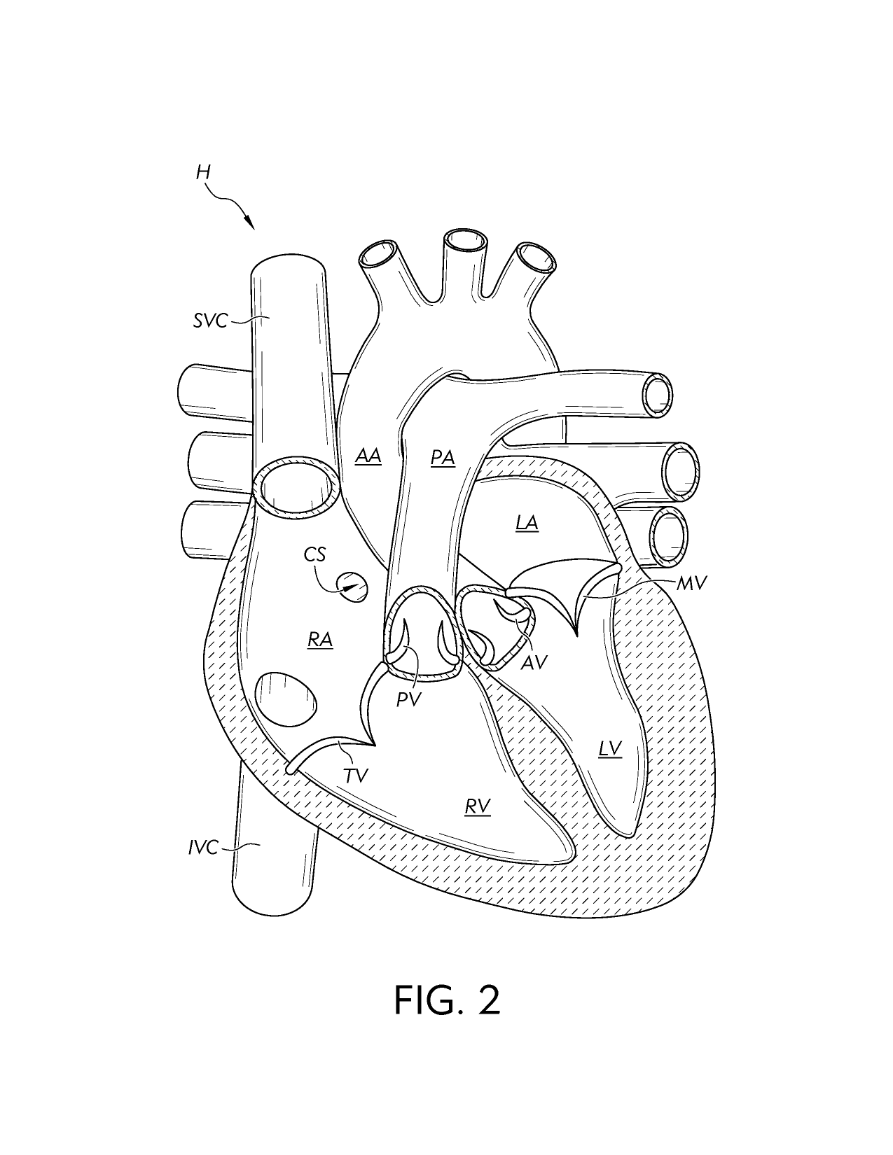

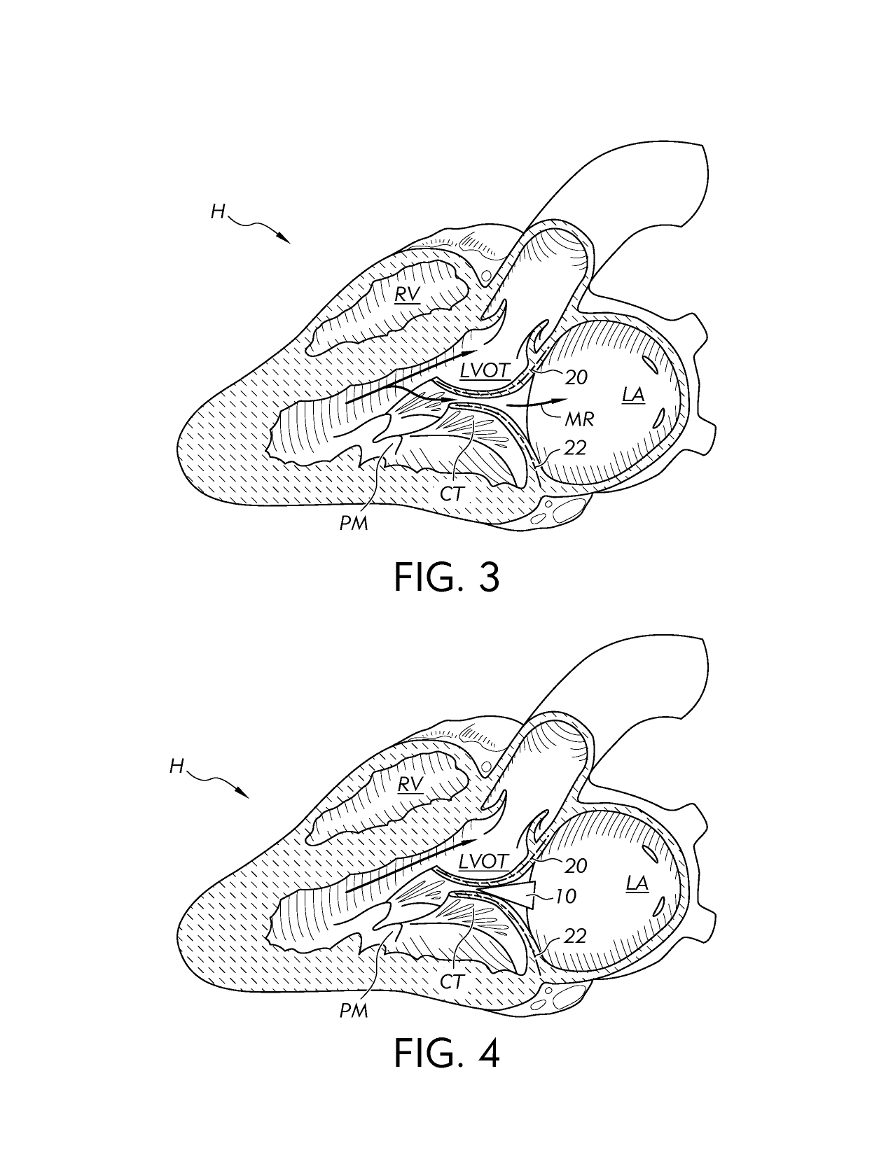

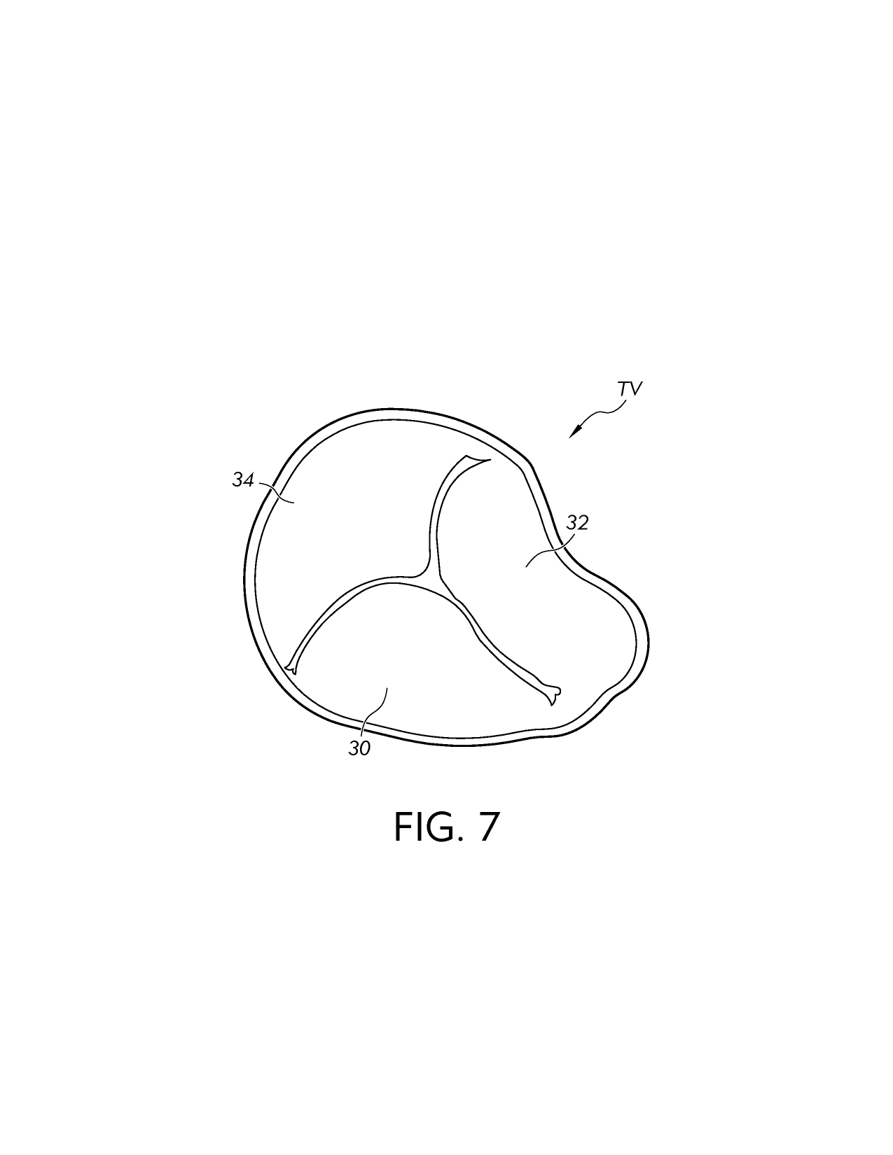

FIGS. 1 and 2 are cutaway views of the human heart H in diastolic and systolic phases, respectively. The right ventricle RV and left ventricle LV are separated from the right atrium RA and left atrium LA, respectively, by the tricuspid valve TV and mitral valve MV; i.e., the atrioventricular valves. Additionally, the aortic valve AV separates the left ventricle LV from the ascending aorta AA, and the pulmonary valve PV separates the right ventricle from the pulmonary artery PA. Each of these valves has flexible leaflets (e.g., leaflets 20, 22 shown in FIGS. 3–6 and leaflets 30, 32, 34 shown in FIG. 7) extending inward across the respective orifices that come together or “coapt” in the flow stream to form the one-way, fluid-occluding surfaces. The native valve repair and/or treatment systems of the present application are frequently described and/or illustrated with respect to the mitral valve MV. Therefore, anatomical structures of the left atrium LA and left ventricle LV will be explained in greater detail. However, the devices described herein can also be used in repairing other native valves, e.g., the devices can be used in repairing the tricuspid valve TV, the aortic valve AV, and the pulmonary valve PV.

The left atrium LA receives oxygenated blood from the lungs. During the diastolic phase, or diastole, seen in FIG. 1, the blood that was previously collected in the left atrium LA (during the systolic phase) moves through the mitral valve MV and into the left ventricle LV by expansion of the left ventricle LV. In the systolic phase, or systole, seen in FIG. 2, the left ventricle LV contracts to force the blood through the aortic valve AV and ascending aorta AA into the body. During systole, the leaflets of the mitral valve MV close to prevent the blood from regurgitating from the left ventricle LV and back into the left atrium LA and blood is collected in the left atrium from the pulmonary vein. In some implementations, the devices described by the present application are used to repair the function of a defective mitral valve MV. That is, the devices are configured to help close the leaflets of the mitral valve to prevent, inhibit or reduce blood from regurgitating from the left ventricle LV and back into the left atrium LA. Many of the devices described in the present application are designed to easily grasp and secure the native leaflets around a coaptation element or spacer that beneficially acts as a filler in the regurgitant orifice to prevent or inhibit back flow or regurgitation during systole, though this is not necessary.

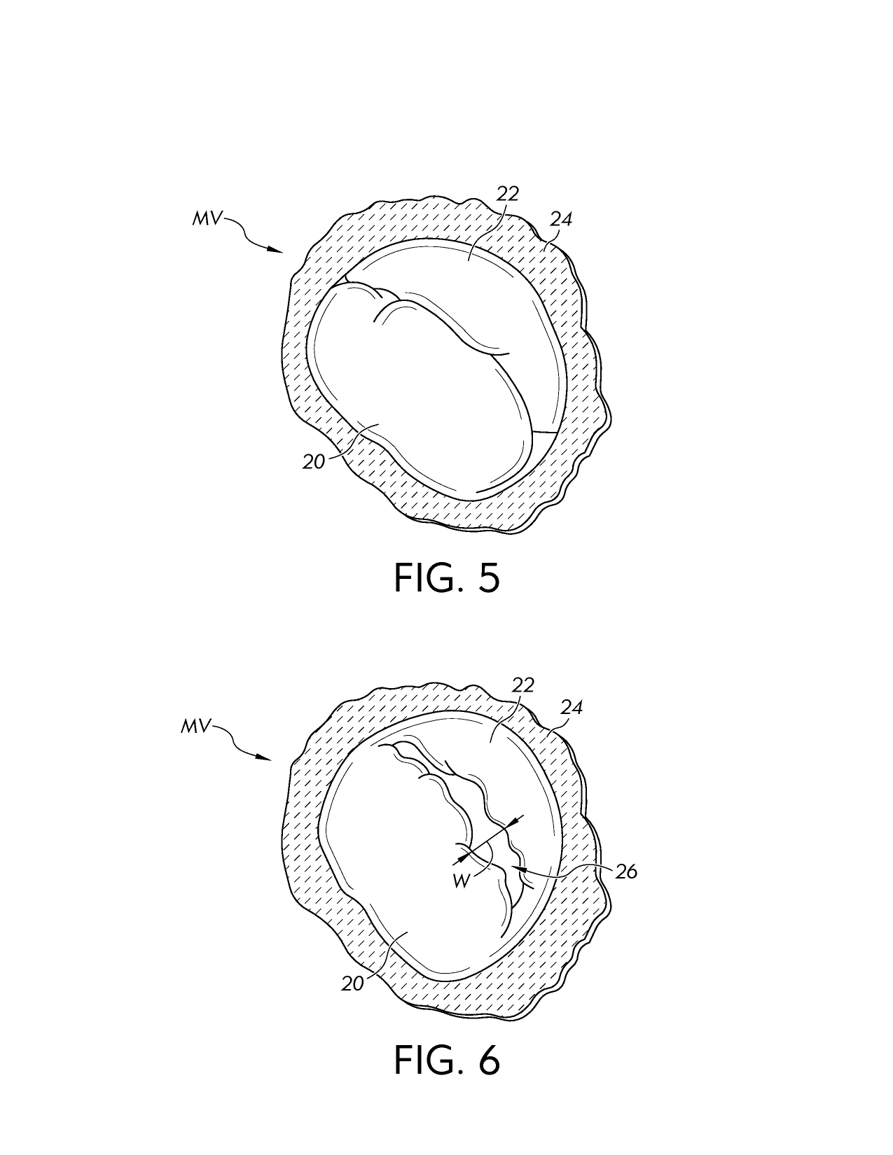

Referring now to FIGS. 1–7, the mitral valve MV includes two leaflets, the anterior leaflet 20 and the posterior leaflet 22. The mitral valve MV also includes an annulus 24 (see FIG. 5), which is a variably dense fibrous ring of tissues that encircles the leaflets 20, 22. Referring to FIGS. 3 and 4, the mitral valve MV is anchored to the wall of the left ventricle LV by chordae tendineae CT. The chordae tendineae CT are cord-like tendons that connect the papillary muscles PM (i.e., the muscles located at the base of the chordae tendineae CT and within the walls of the left ventricle LV) to the leaflets 20, 22 of the mitral valve MV. The papillary muscles PM serve to limit the movements of leaflets 20, 22 of the mitral valve MV and prevent the mitral valve MV from being reverted. The mitral valve MV opens and closes in response to pressure changes in the left atrium LA and the left ventricle LV. The papillary muscles PM do not open or close the mitral valve MV. Rather, the papillary muscles PM support or brace the leaflets 20, 22 against the high pressure needed to circulate blood throughout the body. Together the papillary muscles PM and the chordae tendineae CT are known as the subvalvular apparatus, which functions to keep the mitral valve MV from prolapsing into the left atrium LA when the mitral valve closes. As seen from a Left Ventricular Outflow Tract (LVOT) view shown in FIG. 3, the anatomy of the leaflets 20, 22 is such that the inner sides of the leaflets coapt at the free end portions and the leaflets 20, 22 start receding or spreading apart from each other. The leaflets 20, 22 spread apart in the atrial direction, until each leaflet meets with the mitral annulus.

Various disease processes can impair proper function of one or more of the native valves of the heart H. These disease processes include degenerative processes (e.g., Barlow’s Disease, fibroelastic deficiency, etc.), inflammatory processes (e.g., Rheumatic Heart Disease), and infectious processes (e.g., endocarditis, etc.). In addition, damage to the left ventricle LV or the right ventricle RV from prior heart attacks (i.e., myocardial infarction secondary to coronary artery disease) or other heart diseases (e.g., cardiomyopathy, etc.) may distort a native valve’s geometry, which may cause the native valve to dysfunction. However, the majority of patients undergoing valve surgery, such as surgery to the mitral valve MV, suffer from a degenerative disease that causes a malfunction in a leaflet (e.g., leaflets 20, 22) of a native valve (e.g., the mitral valve MV), which results in prolapse and regurgitation.

Generally, a native valve may malfunction in different ways: including (1) valve stenosis; and (2) valve regurgitation. Valve stenosis occurs when a native valve does not open completely and thereby causes an obstruction of blood flow. Typically, valve stenosis results from buildup of calcified material on the leaflets of a valve, which causes the leaflets to thicken and impairs the ability of the valve to fully open to permit forward blood flow. Valve regurgitation occurs when the leaflets of the valve do not close completely thereby causing blood to leak back into the prior chamber (e.g., causing blood to leak from the left ventricle to the left atrium).

There are three main mechanisms by which a native valve becomes regurgitant—or incompetent—which include Carpentier’s type I, type II, and type III malfunctions. A Carpentier type I malfunction involves the dilation of the annulus such that normally functioning leaflets are distracted from each other and fail to form a tight seal (i.e., the leaflets do not coapt properly). Included in a type I mechanism malfunction are perforations of the leaflets, as are present in endocarditis. A Carpentier’s type II malfunction involves prolapse of one or more leaflets of a native valve above a plane of coaptation. A Carpentier’s type III malfunction involves restriction of the motion of one or more leaflets of a native valve such that the leaflets are abnormally constrained below the plane of the annulus. Leaflet restriction may be caused by rheumatic disease or dilation of a ventricle.

Referring to FIG. 5, when a healthy mitral valve MV is in a closed position, the anterior leaflet 20 and the posterior leaflet 22 coapt, which prevents blood from leaking from the left ventricle LV to the left atrium LA. Referring to FIGS. 3 and 6, mitral regurgitation MR occurs when the anterior leaflet 20 and/or the posterior leaflet 22 of the mitral valve MV is displaced into the left atrium LA during systole so that the edges of the leaflets 20, 22 are not in contact with each other. This failure to coapt causes a gap 26 between the anterior leaflet 20 and the posterior leaflet 22, which allows blood to flow back into the left atrium LA from the left ventricle LV during systole, as illustrated by the mitral regurgitation MR flow path shown in FIG. 3. Referring to FIG. 6, the gap 26 may have a width W between about 2.5 mm and about 17.5 mm, between about 5 mm and about 15 mm, between about 7.5 mm and about 12.5 mm, or about 10 mm. In some situations, the gap 26 may have a width W greater than 15 mm or even 17.5 mm. As set forth above, there are several different ways that a leaflet (e.g., leaflets 20, 22 of mitral valve MV) may malfunction which may thereby lead to valvular regurgitation.

In any of the above-mentioned situations, a device or implant is desired that is capable of engaging the anterior leaflet 20 and the posterior leaflet 22 to close the gap 26 and prevent or inhibit regurgitation of blood through the mitral valve MV. As can be seen in FIG. 4, an abstract representation of a repair or treatment device 10 (e.g., a valve treatment device, a valve repair device, an implantable device, an implant, etc.) is shown implanted between the leaflets 20, 22 such that regurgitation does not occur during systole (compare FIG. 3 with FIG. 4). In some implementations, the coaptation element (e.g., spacer, coaption element, gap filler, membrane, sheet, plug, wedge, balloon, etc.) of the device 10 has a generally tapered or triangular shape that naturally adapts to the native valve geometry and to its expanding leaflet nature (toward the annulus). In this application, the terms spacer, coaption element, coaptation element, gap filler, plug, etc. are used interchangeably and refer to an element that fills a portion of the space between native valve leaflets and/or that is configured such that the native valve leaflets engage or “coapt” against (e.g., such that the native leaflets coapt against the coaption element, coaptation element, spacer, etc. instead of only against one another).

Although stenosis or regurgitation may affect any valve, stenosis is predominantly found to affect either the aortic valve AV or the pulmonary valve PV, and regurgitation is predominantly found to affect either the mitral valve MV or the tricuspid valve TV. Both valve stenosis and valve regurgitation increase the workload of the heart H and may lead to very serious conditions if left un-treated; such as endocarditis, congestive heart failure, permanent heart damage, cardiac arrest, and ultimately death. Because the left side of the heart (i.e., the left atrium LA, the left ventricle LV, the mitral valve MV, and the aortic valve AV) are primarily responsible for circulating the flow of blood throughout the body. Accordingly, because of the substantially higher pressures on the left side heart dysfunction of the mitral valve MV or the aortic valve AV is particularly problematic and often life threatening.

Malfunctioning native heart valves can either be repaired or replaced. Repair typically involves the preservation and correction of the patient’s native valve. Replacement typically involves replacing the patient’s native valve with a biological or mechanical substitute. Typically, the aortic valve AV and pulmonary valve PV are more prone to stenosis. Because stenotic damage sustained by the leaflets is irreversible, treatments for a stenotic aortic valve or stenotic pulmonary valve can be removal and replacement of the valve with a surgically implanted heart valve, or displacement of the valve with a transcatheter heart valve. The mitral valve MV and the tricuspid valve TV are more prone to deformation of leaflets and/or surrounding tissue, which, as described above, may prevent the mitral valve MV or tricuspid valve TV from closing properly and allows for regurgitation or back flow of blood from the ventricle into the atrium (e.g., a deformed mitral valve MV may allow for regurgitation or back flow from the left ventricle LV to the left atrium LA as shown in FIG. 3). The regurgitation or back flow of blood from the ventricle to the atrium results in valvular insufficiency. Deformations in the structure or shape of the mitral valve MV or the tricuspid valve TV are often repairable. In addition, regurgitation may occur due to the chordae tendineae CT becoming dysfunctional (e.g., the chordae tendineae CT may stretch or rupture), which allows the anterior leaflet 20 and the posterior leaflet 22 to be reverted such that blood is regurgitated into the left atrium LA. The problems occurring due to dysfunctional chordae tendineae CT can be repaired by repairing the chordae tendineae CT or the structure of the mitral valve MV (e.g., by securing the leaflets 20, 22 at the affected portion of the mitral valve).

The devices and procedures disclosed herein often make reference to repairing the structure of a mitral valve. However, it should be understood that the devices and concepts provided herein can be used to repair any native valve, as well as any component of a native valve. Such devices can be used between the leaflets 20, 22 of the mitral valve MV to prevent or inhibit regurgitation of blood from the left ventricle into the left atrium. With respect to the tricuspid valve TV (FIG. 7), any of the devices and concepts herein can be used between any two of the anterior leaflet 30, septal leaflet 32, and posterior leaflet 34 to prevent or inhibit regurgitation of blood from the right ventricle into the right atrium. In addition, any of the devices and concepts provided herein can be used on all three of the leaflets 30, 32, 34 together to prevent or inhibit regurgitation of blood from the right ventricle to the right atrium. That is, the treatment device, repair devices, implants, etc. provided herein can be centrally located between the three leaflets 30, 32, 34.

An example device (e.g., valve repair device, valve treatment device, implantable device, implant, etc.) can optionally have a coaptation element (e.g., spacer, coaption element, gap filler, membrane, sheet, plug, wedge, balloon, etc.) and at least one anchor (e.g., one, two, three, or more). In some implementations, a device (e.g., a valve repair device, a valve treatment device, an implantable device, an implant, etc.) can have any combination or sub-combination of the features disclosed herein without a coaptation element. When included, the coaptation element (e.g., spacer, coaption element, gap filler, membrane, sheet, plug, wedge, balloon, etc.) is configured to be positioned within the native heart valve orifice to help fill the space between the leaflets and form a more effective seal, thereby reducing or preventing or inhibiting regurgitation described above. The coaptation element can have a structure that is impervious to blood (or that resists blood flow therethrough) and that allows the native leaflets to close around the coaptation element during ventricular systole to block blood from flowing from the left or right ventricle back into the left or right atrium, respectively. The device can be configured to seal against two or three native valve leaflets; that is, the device can be used in the native mitral (bicuspid) and tricuspid valves. The coaptation element is sometimes referred to herein as a spacer because the coaptation element can fill a space between improperly functioning native leaflets (e.g., mitral leaflets 20, 22 or tricuspid leaflets 30, 32, 34) that do not close completely.

The optional coaptation element (e.g., spacer, coaptation element, gap filler, membrane, sheet, plug, wedge, balloon, etc.) can have various shapes. In some implementations, the coaptation element can have an elongated cylindrical shape having a round cross-sectional shape. In some implementations, the coaptation element can have an oval cross-sectional shape, an ovoid cross-sectional shape, a crescent cross-sectional shape, a rectangular cross-sectional shape, or various other non-cylindrical shapes. In some implementations, the coaptation element can have an atrial portion positioned in or adjacent to the atrium, a ventricular or lower portion positioned in or adjacent to the ventricle, and a side surface that extends between the native leaflets. In some implementations configured for use in the tricuspid valve, the atrial or upper portion is positioned in or adjacent to the right atrium, and the ventricular or lower portion is positioned in or adjacent to the right ventricle, and the side surfaces extend between the native tricuspid leaflets.

In some implementations, the anchor can be configured to secure the device to one or both of the native leaflets such that the coaptation element is positioned between the two native leaflets. In some implementations configured for use in the tricuspid valve, the anchor is configured to secure the device to one, two, or three of the tricuspid leaflets such that the coaptation element is positioned between the three native leaflets. In some implementations, the anchor can attach to the coaptation element at a location adjacent the ventricular portion of the coaptation element. In some implementations, the anchor can attach to an actuation element (e.g., an actuation shaft, actuation tube, actuation wire, etc.) to which the coaptation element is also attached. In some implementations, the anchor and the coaptation element can be positioned independently with respect to each other by separately moving each of the anchor and the coaptation element along the longitudinal axis of the actuation element (e.g., actuation shaft, actuation rod, actuation tube, actuation wire, etc.). In some implementations, the anchor and the coaptation element can be positioned simultaneously by moving the anchor and the coaptation element together along the longitudinal axis of the actuation element (e.g., shaft, actuation wire, etc.). The anchor can be configured to be positioned behind a native leaflet when deployed such that the leaflet is grasped by the anchor.

The device can be configured to be deployed and/or implanted via a delivery system or other means for delivery. The delivery system can comprise one or more of a guide/delivery sheath, a delivery catheter, a steerable catheter, an implant catheter, tube, combinations of these, etc. The coaptation element and the anchor can be compressible to a radially compressed state and can be self-expandable to a radially expanded state when compressive pressure is released. The device can be configured for the anchor to be expanded radially away from the still compressed coaptation element initially in order to create a gap between the coaptation element and the anchor. A native leaflet can then be positioned in the gap. The coaptation element can be expanded radially, closing the gap between the coaptation element and the anchor and capturing the leaflet between the coaptation element and the anchor. In some implementations, the anchor and coaptation element are optionally configured to self-expand. The implantation and/or deployment methods for some implementations can be different and are more fully discussed below with respect to each implementation. Additional information regarding these and other delivery methods that can be used with the concepts herein can be found in U.S. Pat. No. 8,449,599 and U.S. Patent Application Publication Nos. 2014/0222136, 2014/0067052, 2016/0331523, PCT patent application publication Nos. WO2020/076898, WO2023/278663, WO2023/004098, WO2023/091520, WO2023/107296, WO2023/086340, WO2023/003755, and WO2022/231889 each of which is incorporated herein by reference in its entirety for all purposes. These method(s) can be performed on a living animal or on a simulation, such as on a cadaver, cadaver heart, simulator (e.g., with the body parts, heart, tissue, etc. being simulated), etc. mutatis mutandis.

The disclosed devices or implants can be configured such that the anchor is connected to a leaflet, taking advantage of the tension from native chordae tendineae to resist high systolic pressure urging the device toward the left atrium. During diastole, the devices can rely on the compressive and retention forces exerted on the leaflet that is grasped by the anchor.

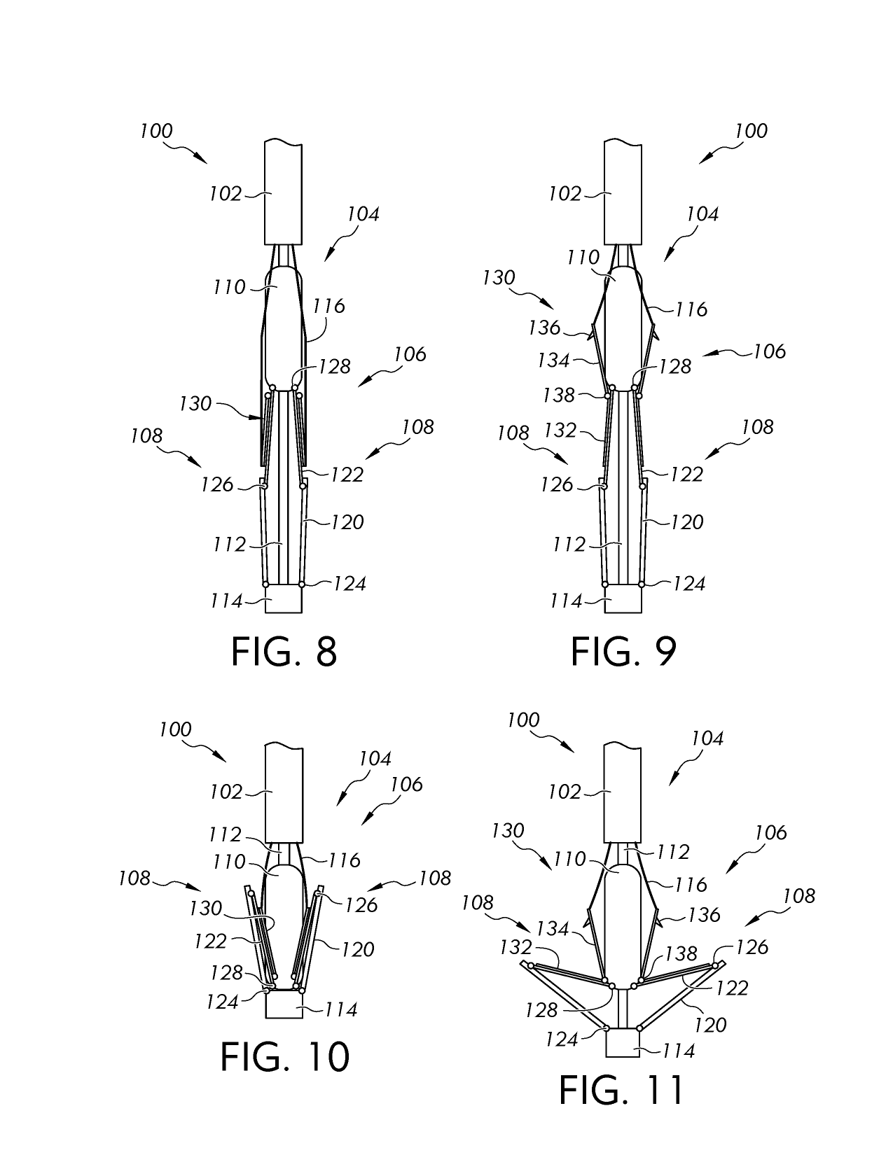

Referring now to FIGS. 8–15, a schematically illustrated device 100 (e.g., a prosthetic device, a valve repair device, valve treatment device, implantable device, implant, etc.) is shown in various stages of deployment. The device 100 and other similar devices and/or implants are described in more detail in PCT patent application publication Nos. WO2018/195215, WO2020/076898, WO2019/139904, WO2023278663, WO2023/004098, WO2023/091520, WO2023/107296, WO2023/086340, WO2023/003755, and WO2022/231889, which are incorporated herein by reference in their entirety for all purposes. The devices herein can include any other features for another device or implant discussed in the present application or the applications cited above, and the devices herein can be positioned to engage valve tissue (e.g., leaflets 20, 22, 30, 32, 34) as part of any suitable treatment and/or repair system (e.g., any valve repair system and/or valve treatment system disclosed in the present application or the applications cited above).

The device 100 is deployed from a delivery system 102. The delivery system 102 can comprise one or more of a catheter, a sheath, a guide catheter/sheath, a delivery catheter/sheath, a steerable catheter, an implant catheter, a tube, a channel, a pathway, combinations of these, etc. The device 100 includes a coaptation portion 104 and an anchor portion 106.

In some implementations, the coaptation portion 104 of the device 100 includes a coaptation element 110 that is adapted to be deployed and/or implanted between leaflets of a native valve (e.g., a native mitral valve, native tricuspid valve, etc.) and is slidably attached to an actuation element 112 (e.g., actuation wire, shaft, tube, hypotube, line, suture, braid, etc.). The anchor portion 106 includes one or more anchors 108 that are actuatable between open and closed conditions and can take a wide variety of forms, such as, for example, paddles, gripping elements, or the like. Actuation of the actuation element 112 opens and closes the anchor portion 106 of the device 100 to grasp the native valve leaflets during deployment and/or implantation. The actuation element 112 (as well as other actuation elements disclosed herein) can take a wide variety of different forms (e.g., as a wire, rod, shaft, tube, screw, suture, line, strip, combination of these, etc.), be made of a variety of different materials, and have a variety of configurations. As one example, the actuation element can be threaded such that rotation of the actuation element moves the anchor portion 106 relative to the coaptation portion 104. Or, the actuation element can be unthreaded, such that pushing or pulling the actuation element 112 moves the anchor portion 106 relative to the coaptation portion 104.

The anchor portion 106 and/or anchors of the device 100 include outer paddles 120 and inner paddles 122 that are, in some implementations, connected between a cap 114 and a coaptation element 110 by portions 124, 126, 128. The portions 124, 126, 128 can be jointed and/or flexible to move between all of the positions described below. The interconnection of the outer paddles 120, the inner paddles 122, the coaptation element 110, and the cap 114 by the portions 124, 126, and 128 can constrain the device to the positions and movements illustrated herein.

In some implementations, the delivery system 102 includes a steerable catheter, implant catheter, and the actuation element 112 (e.g., actuation wire, shaft, tube, hypotube, line, suture, braid, etc.). These can be configured to extend through a guide catheter/sheath (e.g., a transseptal sheath, etc.). In some implementations, the actuation element 112 extends through a delivery catheter and the coaptation element 110 to the distal end (e.g., a cap 114 or other attachment portion at the distal connection of the anchor portion 106). Extending and retracting the actuation element 112 increases and decreases the spacing between the coaptation element 110 and the distal end of the device (e.g., the cap 114 or other attachment portion), respectively. In some implementations, a collar or other attachment element (e.g., clamp, clip, lock, sutures, friction fit, buckle, snap fit, lasso, etc.) removably attaches the coaptation element 110 to the delivery system 102, either directly or indirectly, so that the actuation element 112 slides through the collar or other attachment element and, in some implementations, through a coaptation element 110 during actuation to open and close the paddles 120, 122 of the anchor portion 106 and/or anchors 108.

In some implementations, the anchor portion 106 and/or anchors 108 can include attachment portions or gripping members (e.g., gripping arms, clasp arms, etc.). The illustrated gripping members can comprise clasps 130 that include a base or fixed arm 132, a moveable arm 134, optional friction-enhancing elements, other securing structures 136 (e.g., barbs, protrusions, ridges, grooves, textured surfaces, adhesive, etc.), and a joint portion 138. The fixed arms 132 are attached to the inner paddles 122. In some implementations, the fixed arms 132 are attached to the inner paddles 122 with the joint portion 138 disposed proximate the coaptation element 110. The joint portion 138 provides a spring force between the fixed and moveable arms 132, 134 of the clasp 130. The joint portion 138 can be any suitable joint, such as a flexible joint, a spring joint, a pivot joint, or the like. In some implementations, the joint portion 138 is a flexible piece of material integrally formed with the fixed and moveable arms 132, 134. The fixed arms 132 are attached to the inner paddles 122 and remain stationary or substantially stationary relative to the inner paddles 122 when the moveable arms 134 are opened to open the clasps 130 and expose the optional barbs or other friction-enhancing elements 136.

In some implementations, the clasps 130 are opened by applying tension to actuation lines 116 attached to the moveable arms 134, thereby causing the moveable arms 134 to articulate, flex, or pivot on the joint portions 138. The actuation lines 116 extend through the delivery system 102 (e.g., through a steerable catheter, an implant catheter, etc.). Other actuation mechanisms are also possible.

The actuation line 116 can take a wide variety of forms, such as, for example, a line, a suture, a wire, a rod, a catheter, or the like. The clasps 130 can be spring loaded so that in the closed position the clasps 130 continue to provide a pinching force on the grasped native leaflet. Optional barbs or other friction-enhancing elements 136 of the clasps 130 can grab, pinch, and/or pierce the native leaflets to further secure the native leaflets.

During deployment and/or implantation, the paddles 120, 122 can be opened and closed, for example, to grasp the native leaflets (e.g., native mitral valve leaflets, etc.) between the paddles 120, 122 and/or between the paddles 120, 122 and a coaptation element 110 (e.g., a spacer, plug, membrane, etc.).

The clasps 130 can be used to grasp and/or further secure the native leaflets by engaging the leaflets with optional barbs or other friction-enhancing elements 136 and pinching the leaflets between the moveable and fixed arms 134, 132. The optional barbs or other friction-enhancing elements 136 (e.g., protrusions, ridges, grooves, textured surfaces, adhesive, etc.) of the clasps 130 increase friction with the leaflets or can partially or completely puncture the leaflets.

In some implementations, the actuation lines 116 can be actuated separately (or both separately and simultaneously) so that each clasp 130 can be opened and closed separately. Separate operation allows one leaflet to be grasped at a time, or for the repositioning of a clasp 130 on a leaflet that was insufficiently grasped, without altering a successful grasp on the other leaflet. The clasps 130 can be opened and closed relative to the position of the inner paddle 122 (as long as the inner paddle is in an open or at least partially open position), thereby allowing leaflets to be grasped in a variety of positions as the particular situation requires.

Referring now to FIG. 8, the device 100 is shown in an elongated or fully open condition for deployment from a delivery catheter of the delivery system 102. The device 100 is disposed at the end of the catheter of the delivery system 102 in the fully open position. In the elongated condition the cap 114 is spaced apart from the coaptation element 110 such that the paddles 120, 122 are fully extended. In some implementations, an angle formed between the interior of the outer and inner paddles 120, 122 is approximately 180 degrees. The clasps 130 can be kept in a closed condition during deployment through the delivery system. The actuation lines 116 can extend and attach to the moveable arms 134.

Referring now to FIG. 9, the device 100 is shown in an elongated condition, similar to FIG. 8, but with the clasps 130 in a fully open position, ranging from about 140 degrees to about 200 degrees, from about 170 degrees to about 190 degrees, or about 180 degrees between fixed and moveable arms 132, 134 of the clasps 130.

Referring now to FIG. 10, the device 100 is shown in a shortened or fully closed condition. To move the device 100 from the elongated condition to the shortened condition, the actuation element 112 is retracted to pull the cap 114 towards the coaptation element 110. The connection portion(s) 126 (e.g., joint(s), flexible connection(s), etc.) between the outer paddle 120 and inner paddle 122 are constrained in movement such that compression forces acting on the outer paddle 120 from the cap 114 being retracted towards the coaptation element 110 cause the paddles or gripping elements to move radially outward. During movement from the open position to the closed position, the outer paddles 120 maintain an acute angle with the actuation element 112. The outer paddles 120 can optionally be biased toward a closed position. The inner paddles 122 during the same motion move through a considerably larger angle as they are oriented away from the coaptation element 110 in the open condition and collapse along the sides of the coaptation element 110 in the closed condition.

Referring now to FIGS. 11–13, the device 100 is shown in a partially open, grasp-ready condition. To transition from the fully closed to the partially open condition, the actuation element (e.g., actuation wire, shaft, tube, hypotube, line, suture, braid, etc.) is extended to push the cap 114 away from the coaptation element 110, thereby pulling on the outer paddles 120, which in turn pull on the inner paddles 122, causing the anchors or anchor portion 106 to partially unfold. The actuation lines 116 are also retracted to open the clasps 130 so that the leaflets can be grasped. In some implementations, the pair of inner and outer paddles 122, 120 are moved in unison, rather than independently, by a single actuation element 112. Also, the positions of the clasps 130 are dependent on the positions of the paddles 122, 120. For example, referring to FIG. 10 closing the paddles 122, 120 also closes the clasps. In some implementations, the paddles 120, 122 can be independently controllable. In the example illustrated by FIG. 15, the device 100 can have two actuation elements 111, 113 and two independent caps 115, 117 (or other attachment portions), such that one independent actuation element (e.g., actuation wire, shaft, tube, hypotube, line, suture, braid, etc.) and cap (or other attachment portion) are used to control one paddle, and the other independent actuation element and cap (or other attachment portion) are used to control the other paddle.

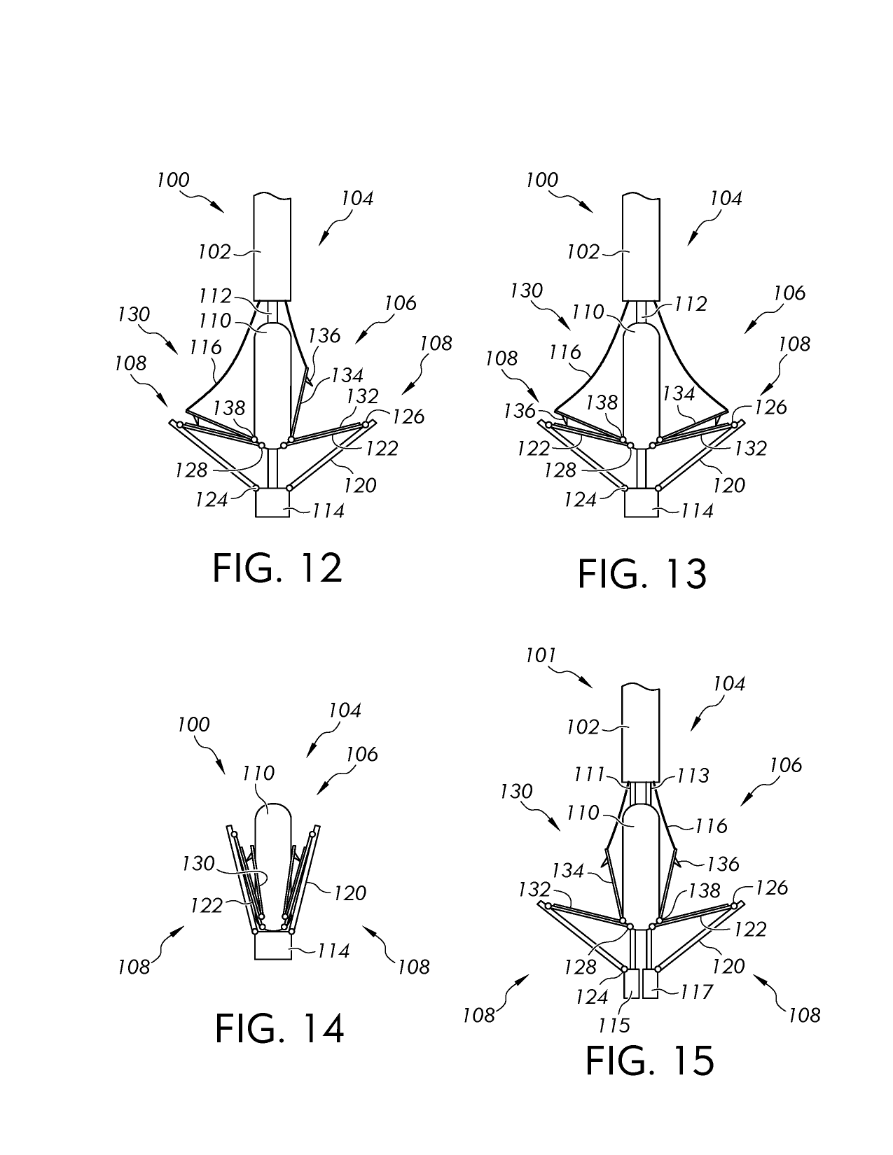

Referring now to FIG. 12, one of the actuation lines 116 is extended to allow one of the clasps 130 to close. Referring now to FIG. 13, the other actuation line 116 is extended to allow the other clasp 130 to close. Either or both of the actuation lines 116 can be repeatedly actuated to repeatedly open and close the clasps 130.

Referring now to FIG. 14, the device 100 is shown in a fully closed and deployed condition. The delivery system 102 and actuation element 112 are retracted and the paddles 120, 122 and clasps 130 remain in a fully closed position. Once deployed, the device 100 can be maintained in the fully closed position with a mechanical latch or can be biased to remain closed through the use of spring materials, such as steel, other metals, plastics, composites, etc. or shape-memory alloys such as Nitinol. For example, the connection portions 124, 126, 128, the joint portions 138, and/or the inner and outer paddles 122, and/or an additional biasing component (not shown) can be formed of metals such as steel or shape-memory alloy, such as Nitinol—produced in a wire, sheet, tubing, or laser sintered powder—and are biased to hold the outer paddles 120 closed around the coaptation element 110 and the clasps 130 pinched around native leaflets. Similarly, the fixed and moveable arms 132, 134 of the clasps 130 are biased to pinch the leaflets. In some implementations, the attachment or connection portions 124, 126, 128, joint portions 138, and/or the inner and outer paddles 122, and/or an additional biasing component (not shown) can be formed of any other suitably elastic material, such as a metal or polymer material, to maintain the device 100 in the closed condition after deployment and/or implantation.

FIG. 15 illustrates an example where the paddles 120, 122 are independently controllable. The device 101 illustrated by FIG. 15 is similar to the device illustrated by FIG. 11, except the device 100 of FIG. 15 includes an actuation element that is configured as two independent actuation elements 111, 113 that are coupled to two independent caps 115, 117. To transition a first inner paddle 122 and a first outer paddle 120 from the fully closed to the partially open condition, the actuation element 111 is extended to push the cap 115 away from the coaptation element 110, thereby pulling on the outer paddle 120, which in turn pulls on the inner paddle 122, causing the first anchor 108 to partially unfold. To transition a second inner paddle 122 and a second outer paddle 120 from the fully closed to the partially open condition, the actuation element 113 is extended to push the cap 115 away from the spacer or coaptation element 110, thereby pulling on the outer paddle 120, which in turn pulls on the inner paddle 122, causing the second anchor 108 to partially unfold. The independent paddle control illustrated by FIG. 15 can be implemented on any of the devices disclosed by the present application. For comparison, in the example illustrated by FIG. 11, the pair of inner and outer paddles 122, 120 are moved in unison, rather than independently, by a single actuation element 112.

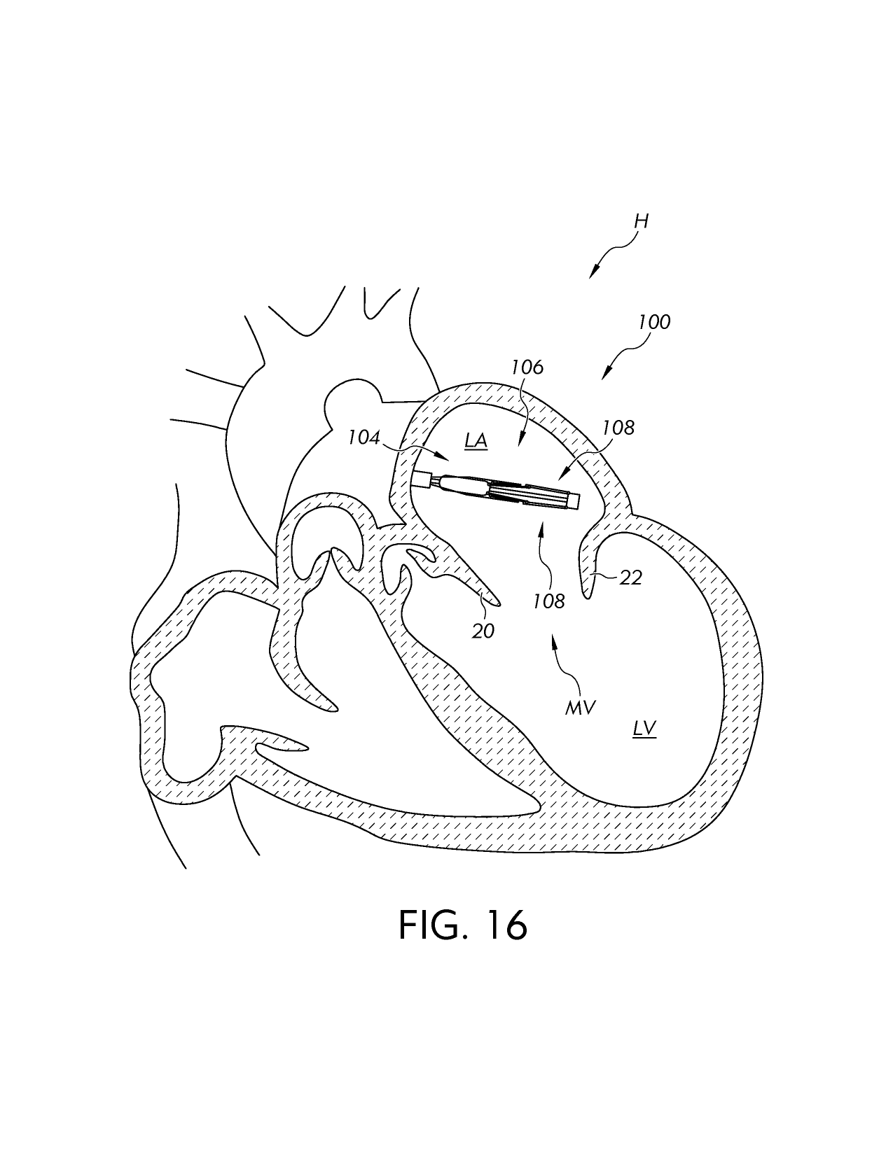

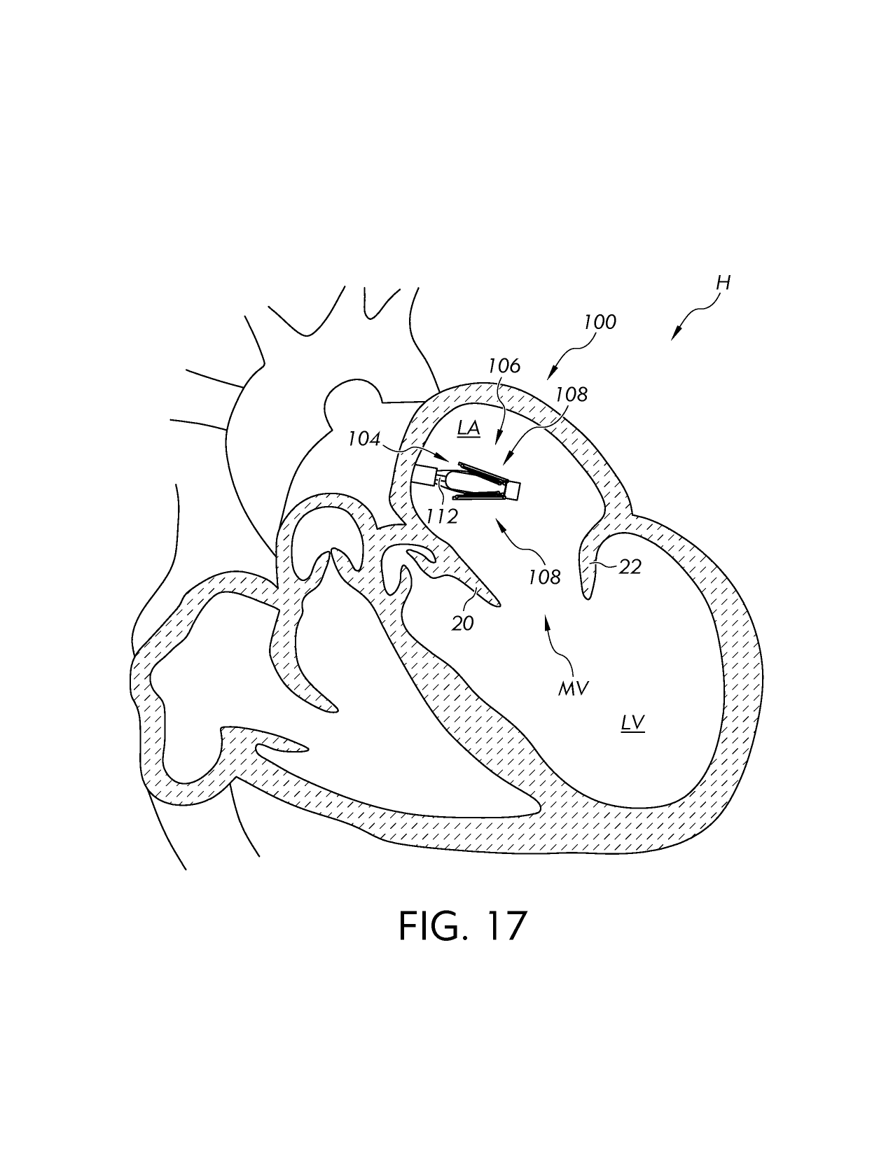

Referring now to FIGS. 16–21, the device 100 of FIGS. 8–14 is shown being delivered and deployed within the native mitral valve MV of the heart H. Referring to FIG. 16, a delivery sheath/catheter is inserted into the left atrium LA through the septum and the implant/device 100 is deployed from the delivery catheter/sheath in the fully open condition as illustrated in FIG. 16. The actuation element 112 is then retracted to move the implant/device into the fully closed condition shown in FIG. 17.

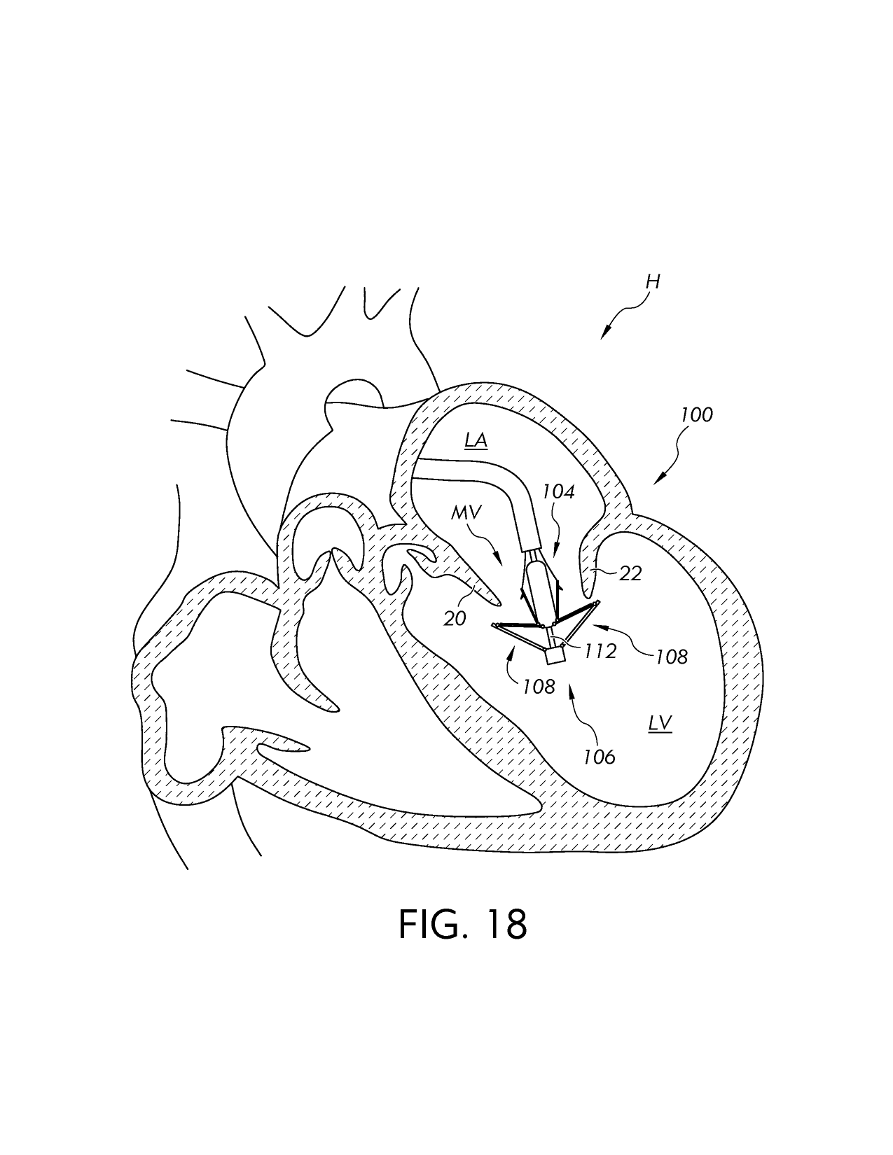

As can be seen in FIG. 18, the implant/device is moved into position within the mitral valve MV into the ventricle LV and partially opened so that the leaflets 20, 22 can be grasped. For example, a steerable catheter can be advanced and steered or flexed to position the steerable catheter as illustrated by FIG. 18. The device or implant catheter connected to the implant/device can be advanced from inside the steerable catheter to position the implant as illustrated by FIG. 18.

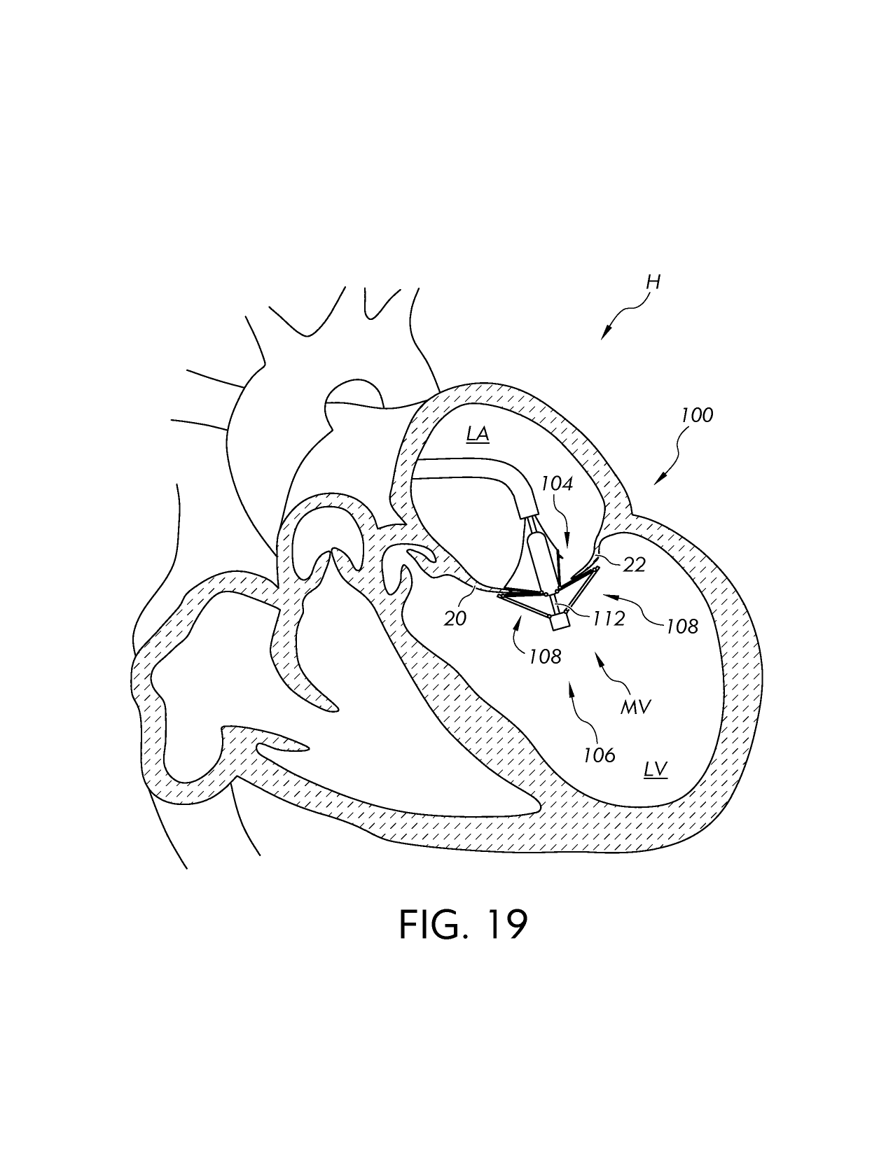

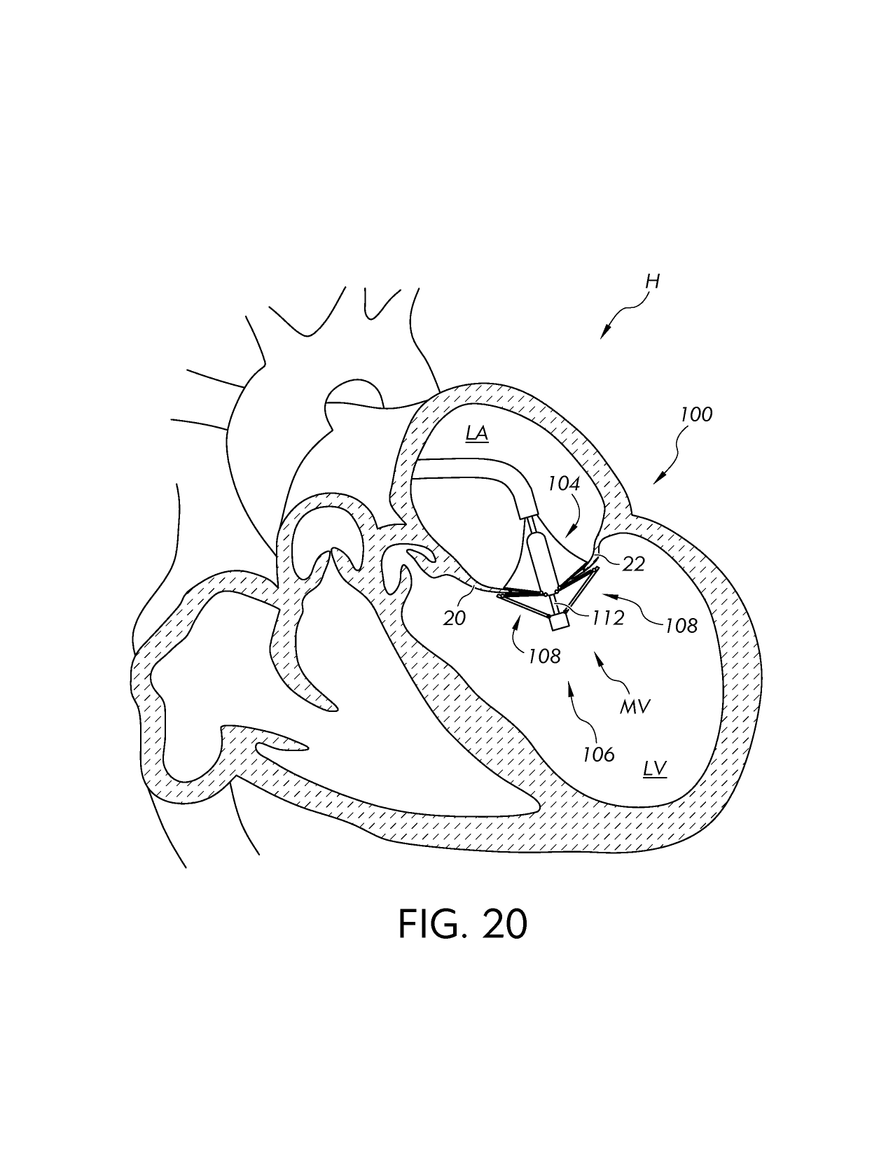

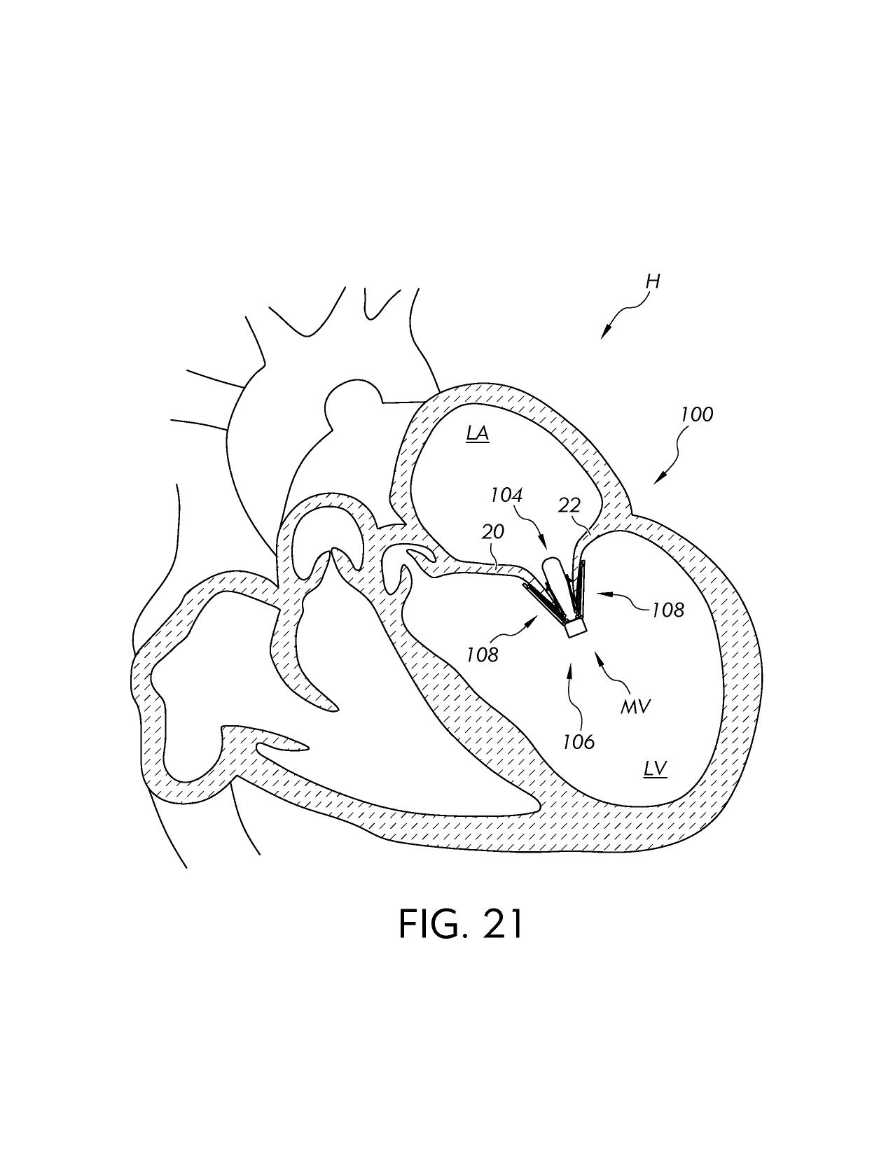

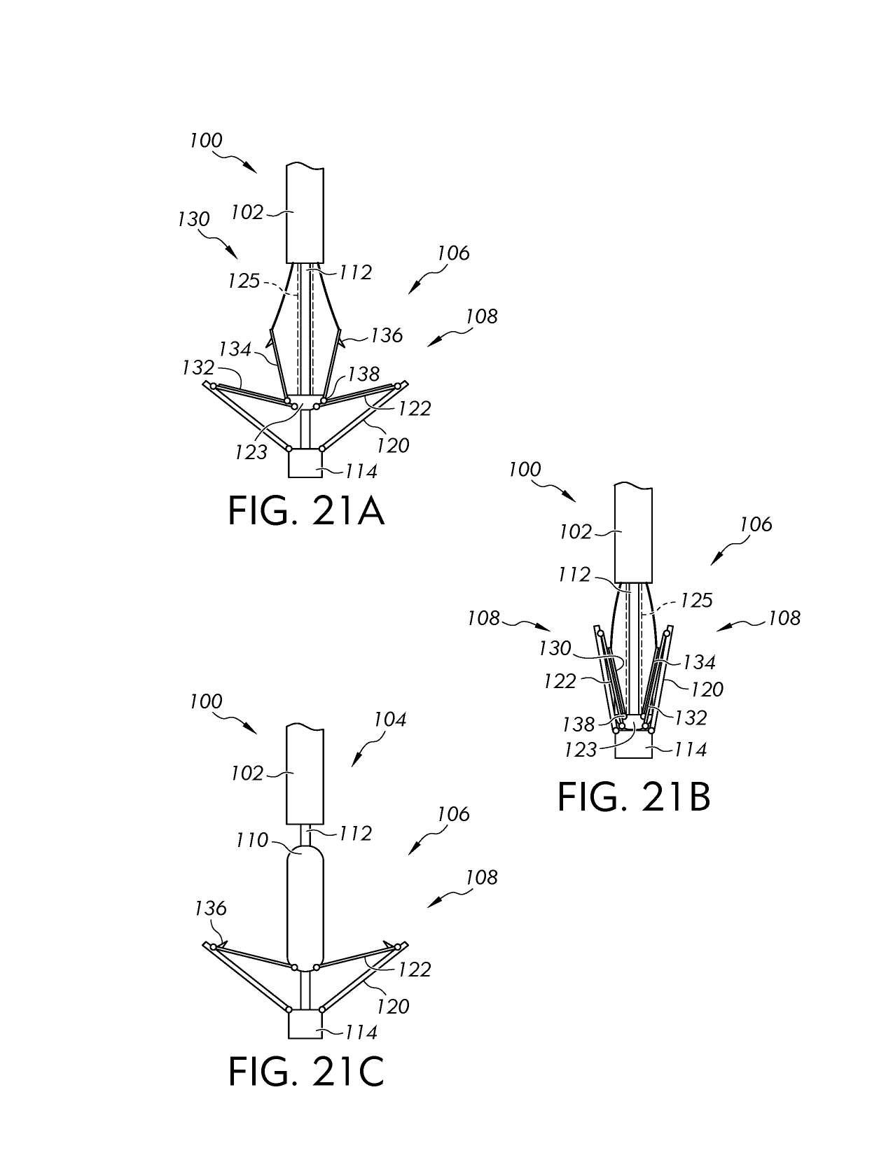

Referring now to FIG. 19, the device catheter can be retracted into the steerable catheter to position the mitral valve leaflets 20, 22 in the clasps 130. An actuation line 116 is extended to close one of the clasps 130, capturing a leaflet 20. FIG. 20 shows the other actuation line 116 being then extended to close the other clasp 130, capturing the remaining leaflet 22. Lastly, as can be seen in FIG. 21, the delivery system 102 (e.g., steerable catheter, implant catheter, etc.), actuation element 112 and actuation lines 116 are then retracted and the device 100 is fully closed and deployed in the native mitral valve MV.

FIG. 21A illustrates a modified version of the device 100 in which the coaptation device 110 illustrated in FIGS. 8-21 is removed. The device 100 illustrated in FIG. 21A can be deployed from the delivery system 102 and includes the anchor portion 106 having outer paddles 120 and inner paddles 122 actuatable between open and closed conditions. In place of the coaptation element 110, the device 100 includes a base 123 and a support structure 125 (e.g., tube, rod, etc.) (illustrated in dashed lines). The outer paddles 120 are connected to the cap 114 and the inner paddles 122 are connected to the outer paddles 120. The actuation element 112 is connected to the cap 114 and is slidable relative to the base 123 and support structure 125. Actuation of the actuation element 112 opens and closes the anchor portion 106 of the device 100 to grasp the native valve leaflets during implantation.

In some implementations, the anchor portion 106 and/or anchors 108 can include attachment portions or gripping members 130 that include a base or fixed arm 132, a moveable arm 134, optional friction-enhancing elements, other securing structures 136 (e.g., barbs, protrusions, ridges, grooves, textured surfaces, adhesive, etc.), and a joint portion 138 attached to the base 123. The base 123 and the support structure 125 can be configured to present a narrower profile (e.g., width) than the illustrated coaptation element 110 of FIGS. 8-21. The narrower width can be advantageous in deployment of the device 100 and withdrawal of the device 100 if desired.

FIG. 21B illustrates a modified version of the device 100 in which the gripping members 130 do not include barbs and the device 100 does not include a coaptation element. The device 100 illustrated in FIG. 21B can be deployed from the delivery system 102 and includes the anchor portion 106 having outer paddles 120 and inner paddles 122 actuatable between open and closed conditions. In place of the coaptation element 110 of FIGS. 8-21, the device 100 includes a base 123 and a support structure 125 (e.g., tube, rod, etc.) (illustrated in dashed lines). The outer paddles 120 are connected to the cap 114 and the inner paddles 122 are connected to the outer paddles 120. The actuation element 112 is connected to the cap 114 and is slidable relative to the base 123 and support structure 125. Actuation of the actuation element 112 opens and closes the anchor portion 106 of the device 100 to grasp the native valve leaflets during implantation.

In some implementations, the attachment portions or gripping members 130 include the base or fixed arm 132, the moveable arm 134, and a joint portion 138 attached to the base 123. The gripping members 130, however, do not include the barbs illustrated in FIG. 11, for example. Thus, in some implementations, the device 100, while able to capture leaflets, is not required to have the same retaining force as other implementations. For example, the device 100 can be used in conjunction with other means for capturing or attaching leaflets. Thus, the device 100 can serve to grab leaflets in a temporary basis while other means (e.g., fusion, anchors, adhesives, etc.) are used for longer term fixation of the leaflets. Further, the base 123 and the support structure 125 can be configured to present narrower profile (e.g., width) than the illustrated coaptation element 110 of FIGS. 8-21. The narrower width can be advantageous in deployment of the device 100 and withdrawal of the device 100 if desired.

FIG. 21C illustrates a modified version of the device 100 in which the gripping members 130 illustrated in FIGS. 8-21 are removed. The device 100 illustrated in FIG. 21C can be deployed from the delivery system 102 and includes the anchor portion 106 having outer paddles 120 and inner paddles 122 actuatable between open and closed conditions. The outer paddles 120 are connected to the cap 114 and the inner paddles 122 are connected to the outer paddles 120 on one end and to the coaptation element 110 on the other end. The actuation element 112 is connected to the cap 114 and is slidable relative to the coaptation element 110. Actuation of the actuation element 112 opens and closes the anchor portion 106 of the device 100 to grasp the native valve leaflets during implantation.

The inner paddles 122 can include optional friction-enhancing elements, other securing structures 136 (e.g., barbs, protrusions, ridges, grooves, textured surfaces, adhesive, etc.). Thus, in a closed condition (e.g., the FIG. 14), the leaflets are pinched between the inner paddles 122 and the coaptation element 110 with the friction-enhancing elements 136 on the inner paddle 122 engaging the leaflets to improve retention of the leaflets by the device 100.

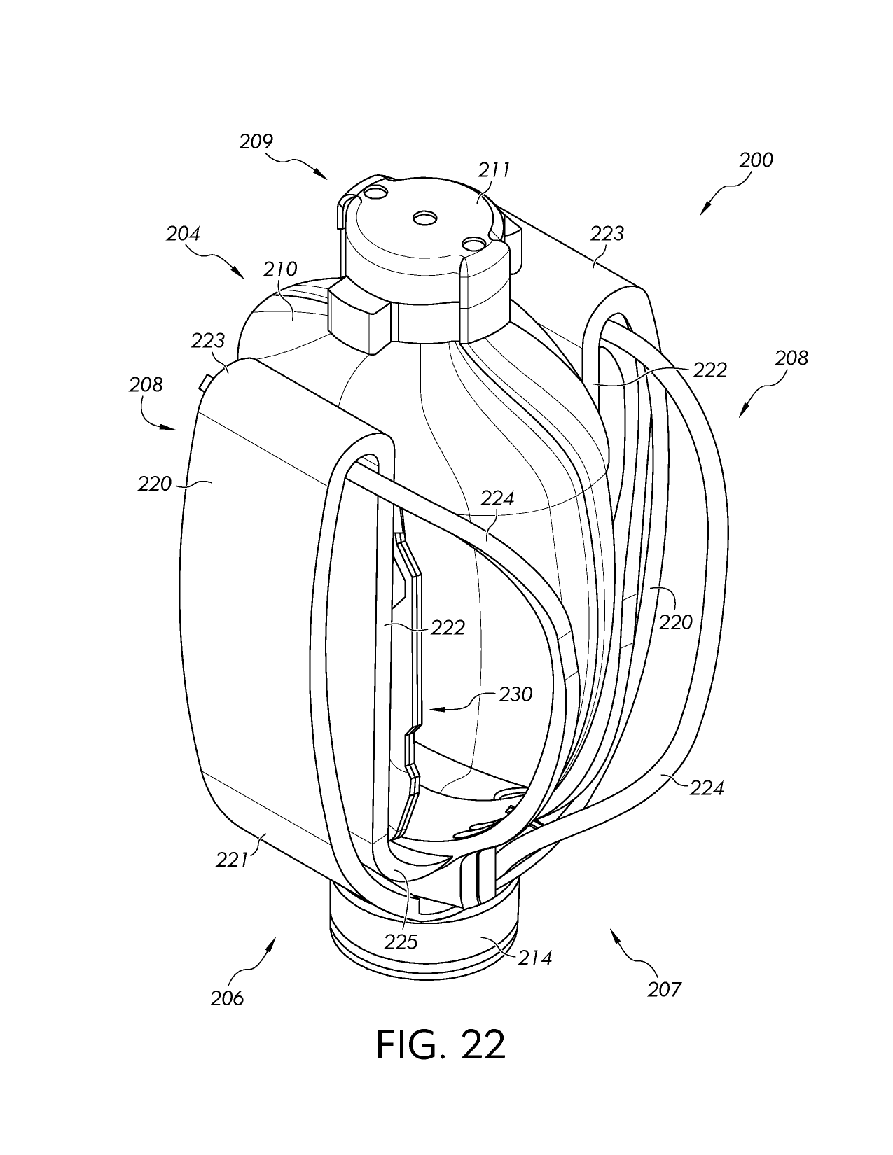

Any of the features disclosed by the present application can be used in a wide variety of different treatment devices and/or repair devices. FIGS. 22-24 illustrate examples of valve treatment and/or repair devices that can be modified to include any of the features disclosed by the present application. Any combination or sub-combination of the features disclosed by the present application can be combined with, substituted for, and/or added to any combination or sub-combination of the features of the devices illustrated by FIGS. 8-24.

Referring now to FIG. 22, an example of a device 200 (e.g., treatment device, repair device, implantable device, implant, etc.) is shown. The device 200 can be configured as an implantable device or implant or other valve treatment device (e.g., one that does not necessarily remain implanted). The device 200 is one of the many different configurations that the device 100 that is schematically illustrated in FIGS. 8–14 can take. The device 200 can include any other features for a device or implant discussed in the present application, and the device 200 can be positioned to engage valve tissue 20, 22 as part of any suitable treatment and/or repair system (e.g., any valve repair system and/or treatment system disclosed in the present application). The device/implant 200 can be a prosthetic spacer device, valve repair device, treatment device, or another type of implant that attaches to leaflets of a native valve.

In some implementations, the device 200 includes a coaptation portion 204, a proximal or attachment portion 209, an anchor portion 206, and a distal portion 207. In some implementations, the coaptation portion 204 of the device optionally includes a coaptation element 210 (e.g., a spacer, coaption element, plug, membrane, sheet, gap filler, plug, wedge, balloon, etc.) for deployment and/or implantation between leaflets of a native valve. In some implementations, the anchor portion 206 includes a plurality of anchors 208. The anchors can be configured in a variety of ways. In some implementations, each anchor 208 includes outer paddles 220, inner paddles 222, paddle extension members or paddle frames 224, and clasps 230. In some implementations, the attachment portion 209 includes a first or proximal collar 211 (or other attachment element) for engaging with a capture mechanism of a delivery system. A delivery system for the device 200 can be the same as or similar to delivery system 102 described above and can comprise one or more of a catheter, a sheath, a guide catheter/sheath, a delivery catheter/sheath, a steerable catheter, an implant catheter, a tube, a channel, a pathway, combinations of these, etc. The capture mechanism can be configured in a variety of ways and, in some implementations, can comprise one or more of a clamp, clip, pin, suture, line, lasso, noose, snare, buckle, lock, latch, etc.

In some implementations, the coaptation element 210 and paddles 220, 222 are formed from a flexible material that can be a metal fabric, such as a mesh, woven, braided, or formed in any other suitable way or a laser cut or otherwise cut flexible material. The material can be cloth, shape-memory alloy wire—such as Nitinol—to provide shape-setting capability, or any other flexible material suitable for deployment and/or implantation in the human body.

An actuation element (e.g., actuation wire, shaft, tube, hypotube, line, suture, braid, etc.) can extend from a delivery system (not shown) to engage and enable actuation of the device or implant 200. In some implementations, the actuation element extends through the proximal collar 211, and spacer or coaptation element 210 to engage a cap 214 of the distal portion 207. The actuation element can be configured to removably engage the cap 214 with a threaded connection, or the like, so that the actuation element can be disengaged and removed from the device 200 after implantation.

The coaptation element 210 extends from the proximal collar 211 (or other attachment element) to the inner paddles 222. In some implementations, the coaptation element 210 has a generally elongated and round shape, though other shapes and configurations are possible. In some implementations, the coaptation element 210 has an elliptical shape or cross-section when viewed from above and has a tapered shape or cross-section when seen from a front view and a round shape or cross-section when seen from a side view. A blend of these three geometries can result in the three-dimensional shape of the illustrated coaptation element 210 that achieves the benefits described herein. The round shape of the coaptation element 210 can also be seen, when viewed from above, to substantially follow or be close to the shape of the paddle frames 224.

The size and/or shape of the coaptation element 210 can be selected to minimize the number of implants that a single patient will require (preferably one), while at the same time maintaining low transvalvular gradients. In some implementations, the anterior-posterior distance at the top of the coaptation element is about 5 mm, and the medial-lateral distance of the coaptation element at its widest is about 10 mm. In some implementations, the overall geometry of the device 200 can be based on these two dimensions and the overall shape strategy described above. It should be readily apparent that the use of other anterior-posterior distance anterior-posterior distance and medial-lateral distance as starting points for the device will result in a device having different dimensions. Further, using other dimensions and the shape strategy described above will also result in a device having different dimensions.

In some implementations, the outer paddles 220 are jointably attached to the cap 214 of the distal portion 207 by connection portions 221 and to the inner paddles 222 by connection portions 223. The inner paddles 222 are jointably attached to the coaptation element by connection portions 225. In this manner, the anchors 208 are configured similar to legs in that the inner paddles 222 are like upper portions of the legs, the outer paddles 220 are like lower portions of the legs, and the connection portions 223 are like knee portions of the legs.

In some implementations, the inner paddles 222 are stiff, relatively stiff, rigid, have rigid portions and/or are stiffened by a stiffening member or a fixed portion of the clasps 230. The inner paddle 222, the outer paddle 220, and the coaptation element can all be interconnected as described herein.

In some implementations, the paddle frames 224 are attached to the cap 214 at the distal portion 207 and extend to the connection portions 223 between the inner and outer paddles 222, 220. In some implementations, the paddle frames 224 are formed of a material that is more rigid and stiff than the material forming the paddles 222, 220 so that the paddle frames 224 provide support for the paddles 222, 220.

The paddle frames 224 can provide additional pinching force between the inner paddles 222 and the coaptation element 210 and assist in wrapping the leaflets around the sides of the coaptation element 210. That is, the paddle frames 224 can be configured with a round three-dimensional shape extending from the cap 214 to the connection portions 223 of the anchors 208. The connections between the paddle frames 224, the outer and inner paddles 220, 222, the cap 214, and the coaptation element 210 can constrain each of these parts to the movements and positions described herein. In particular the connection portion 223 is constrained by its connection between the outer and inner paddles 220, 222 and by its connection to the paddle frame 224. Similarly, the paddle frame 224 is constrained by its attachment to the connection portion 223 (and thus the inner and outer paddles 222, 220) and to the cap 214.

The wide configuration of the paddle frames 224 provides increased surface area compared to the inner paddles 222 alone. The increased surface area can distribute the clamping force of the paddles 220 and paddle frames 224 against the native leaflets over a relatively larger surface of the native leaflets in order to further protect the native leaflet tissue.

Additional features of the device 200, modified versions of the device, delivery systems for the device, and methods for using the device and delivery system are disclosed by Patent Cooperation Treaty International Application No. PCT/US2018/028189 (International Publication No. WO 2018/195215) and the other applications incorporated herein. Any combination or sub-combination of the features disclosed by the present application can be combined with any combination or sub-combination of the features disclosed by Patent Cooperation Treaty International Application No. PCT/US2018/028189 (International Publication No. WO 2018/195215) and/or the other applications incorporated herein. Patent Cooperation Treaty International Application No. PCT/US2018/028189 (International Publication No. WO 2018/195215) is incorporated herein by reference in its entirety.

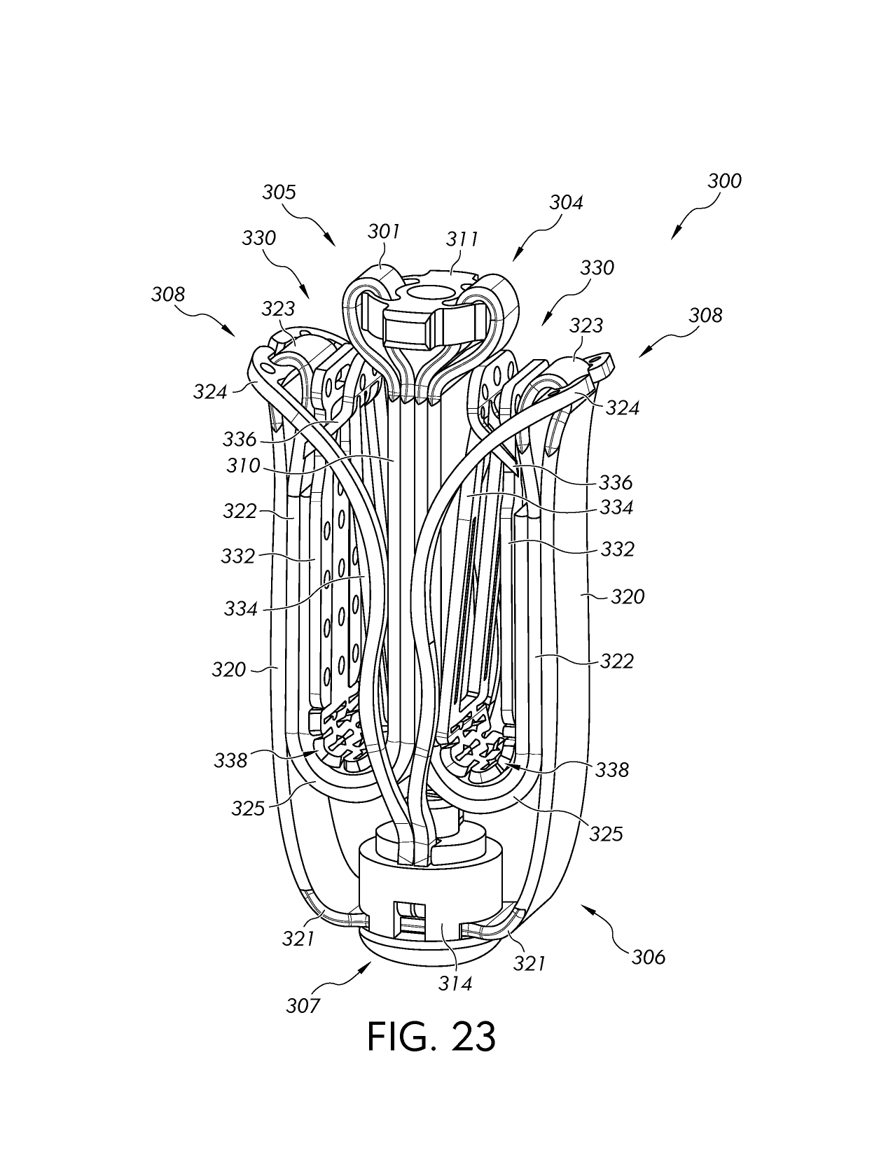

Referring now to FIG. 23, an example of a device 300 (e.g., a valve repair device, a valve treatment device, an implantable device, an implant, etc.) is shown. The device 300 is one of the many different configurations that the device 100 that is schematically illustrated in FIGS. 8–14 can take. The device 300 can include any other features for a device or implant discussed in the present application, and the device 300 can be positioned to engage valve tissue 20, 22 as part of any suitable treatment and/or repair system (e.g., any valve repair system and/or treatment system disclosed in the present application).

The device or implant 300 includes a proximal or attachment portion 305, an anchor portion 306, and a distal portion 307. In some implementations, the device/implant 300 includes a coaptation portion 304, and the coaptation portion 304 can optionally include a coaptation element 310 (e.g., spacer, plug, membrane, sheet, etc.) for deployment and/or implantation between the leaflets 20, 22 of the native valve. In some implementations, the anchor portion 306 includes a plurality of anchors 308. In some implementations, each anchor 308 can include one or more paddles, e.g., outer paddles 320, inner paddles 322, paddle extension members or paddle frames 324. The anchors can also include and/or be coupled to clasps 330. In some implementations, the attachment portion 305 includes a first or proximal collar 311 (or other attachment element) for engaging with a capture mechanism of a delivery system.

The anchors 308 can be attached to the other portions of the device and/or to each other in a variety of different ways (e.g., directly, indirectly, welding, sutures, adhesive, links, latches, integrally formed, a combination of some or all of these, etc.). In some implementations, the anchors 308 are attached to a coaptation element 310 by connection portions 325 and to a cap 314 by connection portions 321.

The anchors 308 can comprise first portions or outer paddles 320 and second portions or inner paddles 322 separated by connection portions 323. The connection portions 323 can be attached to paddle frames 324 that are hingeably attached to a cap 314 or other attachment portion. In this manner, the anchors 308 are configured similar to legs in that the inner paddles 322 are like upper portions of the legs, the outer paddles 320 are like lower portions of the legs, and the connection portions 323 are like knee portions of the legs.

In implementations with a coaptation element 310, the coaptation element 310 and the anchors 308 can be coupled together in various ways. As shown in the illustrated example, the coaptation element 310 and the anchors 308 can be coupled together by integrally forming the coaptation element 310 and the anchors 308 as a single, unitary component. This can be accomplished, for example, by forming the coaptation element 310 and the anchors 308 from a continuous strip 301 of a braided or woven material, such as braided or woven nitinol wire. In the illustrated example, the coaptation element 310, the outer paddle portions 320, the inner paddle portions 322, and the connection portions 321, 323, 325 are formed from a continuous strip 301.

Like the anchors 208 of the device 200 described above, the anchors 308 can be configured to move between various configurations by axially moving the distal end of the device (e.g., cap 314, etc.) relative to the proximal end of the device (e.g., proximal collar 311 or other attachment element, etc.). This movement can be along a longitudinal axis extending between the distal end (e.g., cap 314, etc.) and the proximal end (e.g., collar 311 or other attachment element, etc.) of the device.

In some implementations, in the straight configuration, the paddle portions 320, 322 are aligned or straight in the direction of the longitudinal axis of the device. In some implementations, the connection portions 323 of the anchors 308 are adjacent the longitudinal axis of the spacer or coaptation element 310. From the straight configuration, the anchors 308 can be moved to a fully folded configuration (e.g., FIG. 23), e.g., by moving the proximal end and distal end toward each other and/or toward a midpoint or center of the device.

In some implementations, the clasps comprise a moveable arm coupled to an anchor. In some implementations, the clasps 330 include a base or fixed arm 332, a moveable arm 334, optional barbs/friction-enhancing elements 336, and a joint portion 338. The fixed arms 332 are attached to the inner paddles 322, with the joint portion 338 disposed proximate the coaptation element 310. The joint portion 338 is spring-loaded so that the fixed and moveable arms 332, 334 are biased toward each other when the clasp 330 is in a closed condition.

The fixed arms 332 are attached to the inner paddles 322 through holes or slots with sutures. The fixed arms 332 can be attached to the inner paddles 322 with any suitable means, such as screws or other fasteners, crimped sleeves, mechanical latches or snaps, welding, adhesive, or the like. The fixed arms 332 remain substantially stationary relative to the inner paddles 322 when the moveable arms 334 are opened to open the clasps 330 and expose the optional barbs 336. The clasps 330 are opened by applying tension to actuation lines attached to the moveable arms 334, thereby causing the moveable arms 334 to articulate, pivot, and/or flex on the joint portions 338.

In short, the device 300 is similar in configuration and operation to the device 200 described above, except that the coaptation element 310, outer paddles 320, inner paddles 322, and connection portions 321, 323, 325 are formed from the single strip of material 301. In some implementations, the strip of material 301 is attached to the proximal collar 311, cap 314, and paddle frames 324 by being woven or inserted through openings in the proximal collar 311, cap 314, and paddle frames 324 that are configured to receive the continuous strip of material 301. The continuous strip 301 can be a single layer of material or can include two or more layers. In some implementations, portions of the device 300 have a single layer of the strip of material 301 and other portions are formed from multiple overlapping or overlying layers of the strip of material 301.

For example, FIG. 23 shows a coaptation element 310 and inner paddles 322 formed from multiple overlapping layers of the strip of material 301. The single continuous strip of material 301 can start and end in various locations of the device 300. The ends of the strip of material 301 can be in the same location or different locations of the device 300. For example, in the illustrated example of FIG. 23, the strip of material 301 begins and ends in the location of the inner paddles 322.

As with the device 200 described above, the size of the coaptation element 310 can be selected to minimize the number of implants that a single patient will require (preferably one), while at the same time maintaining low transvalvular gradients. In particular, forming many components of the device 300 from the strip of material 301 allows the device 300 to be made smaller than the device 200. For example, in some implementations, the anterior-posterior distance at the top of the coaptation element 310 is less than 2 mm, and the medial-lateral distance of the device 300 (i.e., the width of the paddle frames 324 which are wider than the coaptation element 310) at its widest is about 5 mm.

Additional features of the device 300, modified versions of the device, delivery systems for the device, and methods for using the device and delivery system are disclosed by Patent Cooperation Treaty International Application No. PCT/US2019/055320 (International Publication No. WO 2020/076898) and/or any other applications incorporated herein. Any combination or sub-combination of the features disclosed by the present application can be combined with any combination or sub-combination of the features disclosed by Patent Cooperation Treaty International Application No. PCT/US2019/055320 (International Publication No. WO 2020/076898) and/or any other applications incorporated herein. Patent Cooperation Treaty International Application No. PCT/US2019/055320 (International Publication No. WO 2020/076898) is incorporated herein by reference in its entirety.

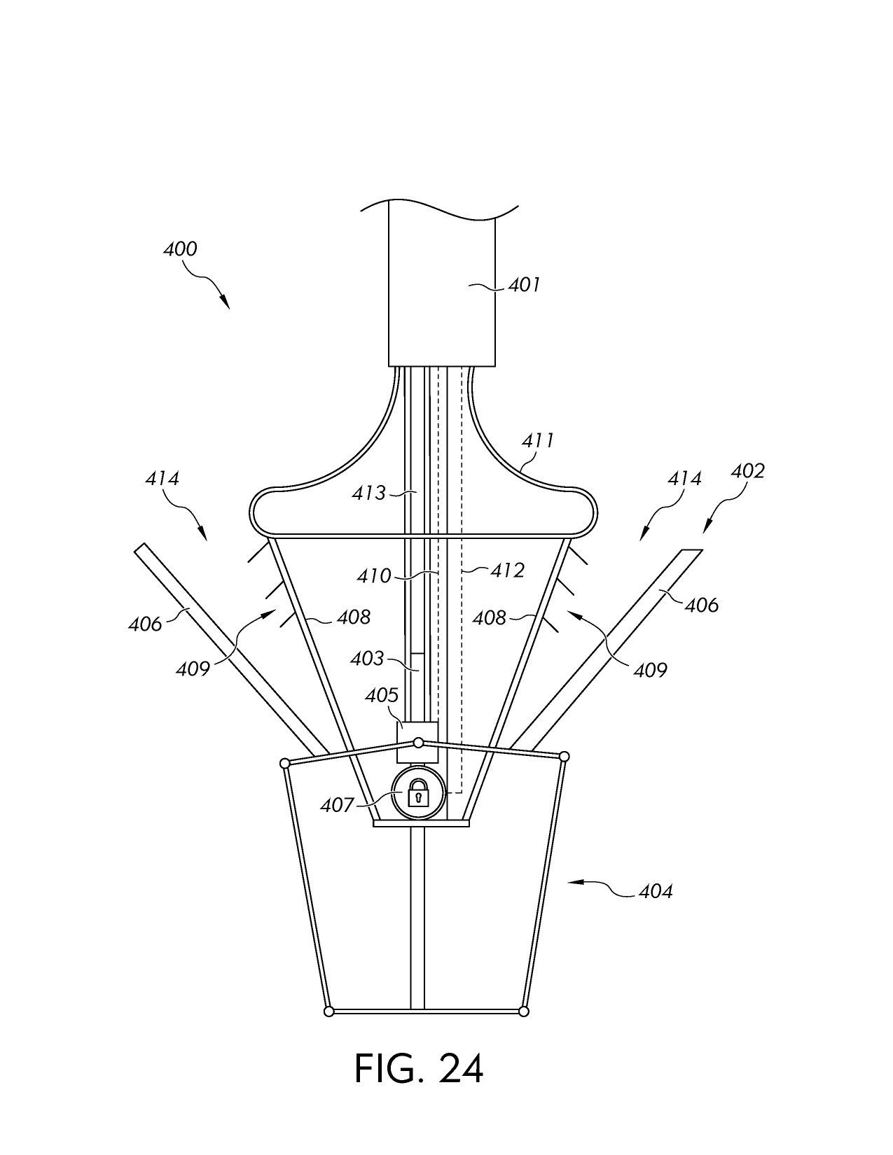

FIG. 24 illustrates an example of one of the many treatment and/or repair systems 400 for treating and/or repairing a native valve of a patient that the concepts of the present application can be applied to. The treatment and/or repair system 400 includes a delivery device 401 and a treatment and/or repair device 402.

In some implementations, the treatment device or repair device 402 includes a base assembly 404, a pair of paddles 406, and a pair of gripping members 408 (e.g., clasps, clasp arms, grippers, gripping arms, latches, etc.). In one example, the paddles 406 can be integrally formed with the base assembly. For example, the paddles 406 can be formed as extensions of links of the base assembly. In the illustrated example, the base assembly 404 of the device 402 has a shaft 403, a coupler 405 configured to move along the shaft, and a lock 407 configured to lock the coupler in a stationary position on the shaft. The coupler 405 is mechanically connected to the paddles 406, such that movement of the coupler 405 along the shaft 403 causes the paddles to move between an open position and a closed position. In this way, the coupler 405 serves as a means for mechanically coupling the paddles 406 to the shaft 403 and, when moving along the shaft 403, for causing the paddles 406 to move between their open and closed positions.

In some implementations, the gripping members 408 are pivotally connected to the base assembly 404 (e.g., the gripping members 408 can be pivotally connected to the shaft 403, or any other suitable member of the base assembly), such that the gripping members can be moved to adjust the width of the opening 414 between the paddles 406 and the gripping members 408. The gripping member 408 can include an optional barbed portion 409 for attaching the gripping members to valve tissue when the device 402 is attached to the valve tissue. When the paddles 406 are in the closed position, the paddles engage the gripping members 408, such that, when valve tissue is attached to the barbed portion 409 of the gripping members, the paddles secure the device 402 to the valve tissue. In some implementations, the gripping members 408 are configured to engage the paddles 406 such that the barbed portion 409 engages the valve tissue member and the paddles 406 to secure the device 402 to the valve tissue member. For example, in certain situations, it can be advantageous to have the paddles 406 maintain an open position and have the gripping members 408 move outward toward the paddles 406 to engage valve tissue and the paddles 406.

While the example shown in FIG. 24 illustrates a pair of paddles 406 and a pair of gripping members 408, it should be understood that the device 402 can include any suitable number of paddles and gripping members.

In some implementations, the system 400 includes a placement shaft 413 that is removably attached to the shaft 403 of the base assembly 404 of the device 402. In some implementations, after the device 402 is secured to valve tissue, the placement shaft 413 can be removed from the shaft 403 to remove the device 402 from the remainder of the treatment and/or repair system 400, such that the device 402 can remain attached to the valve tissue, and the delivery device 401 can be removed from a patient’s body.

The treatment and/or repair system 400 can also include a paddle control mechanism 410, a gripper control mechanism 411, and a lock control mechanism 412. The paddle control mechanism 410 is mechanically attached to the coupler 405 to move the coupler along the shaft, which causes the paddles 406 to move between the open and closed positions. The paddle control mechanism 410 can take any suitable form, and can comprise, for example, a shaft, wire, tube, hypotube, rod, suture, line, etc. For example, the paddle control mechanism can comprise a hollow shaft, a catheter tube or a sleeve that fits over the placement shaft 413 and the shaft 403 and is connected to the coupler 405.

The gripper control mechanism 411 is configured to move the gripping members 408 such that the width of the opening 414 between the gripping members and the paddles 406 can be altered. The gripper control mechanism 411 can take any suitable form, such as, for example, a line, a suture or wire, a rod, a catheter, a tube, a hypotube, etc.

The lock control mechanism 412 is configured to lock and unlock the lock. The lock 407 locks the coupler 405 in a stationary position with respect to the shaft 403 and can take a wide variety of different forms and the type of lock control mechanism 412 can be dictated by the type of lock used. In examples in which the lock 407 includes a pivotable plate, the lock control mechanism 412 is configured to engage the pivotable plate to move the plate between the tilted and substantially non-tilted positions. The lock control mechanism 412 can be, for example, a rod, a suture, a wire, or any other member that is capable of moving a pivotable plate of the lock 407 between a tilted and substantially non-tilted position.

The device 402 is movable from an open position to a closed position. The base assembly 404 includes links that are moved by the coupler 405. The coupler 405 is movably attached to the shaft 403. In order to move the device from the open position to the closed position, the coupler 405 is moved along the shaft 403, which moves the links.

The gripper control mechanism 411 is moves the gripping members 408 to provide a wider or a narrower gap at the opening 414 between the gripping members and the paddles 406. In the illustrated example, the gripper control mechanism 411 includes a line, such as a suture, a wire, etc. that is connected to an opening in an end of the gripping members 408. When the line(s) is pulled, the gripping members 408 move inward, which causes the opening 414 between the gripping members and the paddles 406 to become wider.

In order to move the device 402 from the open position to the closed position, the lock 407 is moved to an unlocked condition by the lock control mechanism 412. Once the lock 407 is in the unlocked condition, the coupler 405 can be moved along the shaft 403 by the paddle control mechanism 410.

After the paddles 406 are moved to the closed position, the lock 407 is moved to the locked condition by the lock control mechanism 412 to maintain the device 402 in the closed position. After the device 402 is maintained in the locked condition by the lock 407, the device 402 is removed from the delivery device 401 by disconnecting the shaft 403 from the placement shaft 413. In addition, the device 402 is disengaged from the paddle control mechanism 410, the gripper control mechanism 411, and the lock control mechanism 412.

Additional features of the device 402, modified versions of the device, delivery systems for the device, and methods for using the device and delivery system are disclosed by Patent Cooperation Treaty International Application No. PCT/US2019/012707 (International Publication No. WO 2019139904) and/or any other applications incorporated herein. Any combination or sub-combination of the features disclosed by the present application can be combined with any combination or sub-combination of the features disclosed by Patent Cooperation Treaty International Application No. PCT/US2019/012707 (International Publication No. WO 2019139904) and/or any other applications incorporated herein. Patent Cooperation Treaty International Application No. PCT/US2019/012707 (International Publication No. WO 2019139904) is incorporated herein by reference in its entirety.

Clasps or leaflet gripping devices disclosed herein can take a wide variety of different forms. Examples of clasps are disclosed by Patent Cooperation Treaty International Application No. PCT/US2018/028171 (International Publication No. WO 2018195201). Any combination or sub-combination of the features disclosed by the present application can be combined with any combination or sub-combination of the features disclosed by Patent Cooperation Treaty International Application No. PCT/US2018/028171 (International Publication No. WO 2018195201). Patent Cooperation Treaty International Application No. PCT/US2018/028171 (International Publication No. WO 2018195201) is incorporated herein by reference in its entirety.

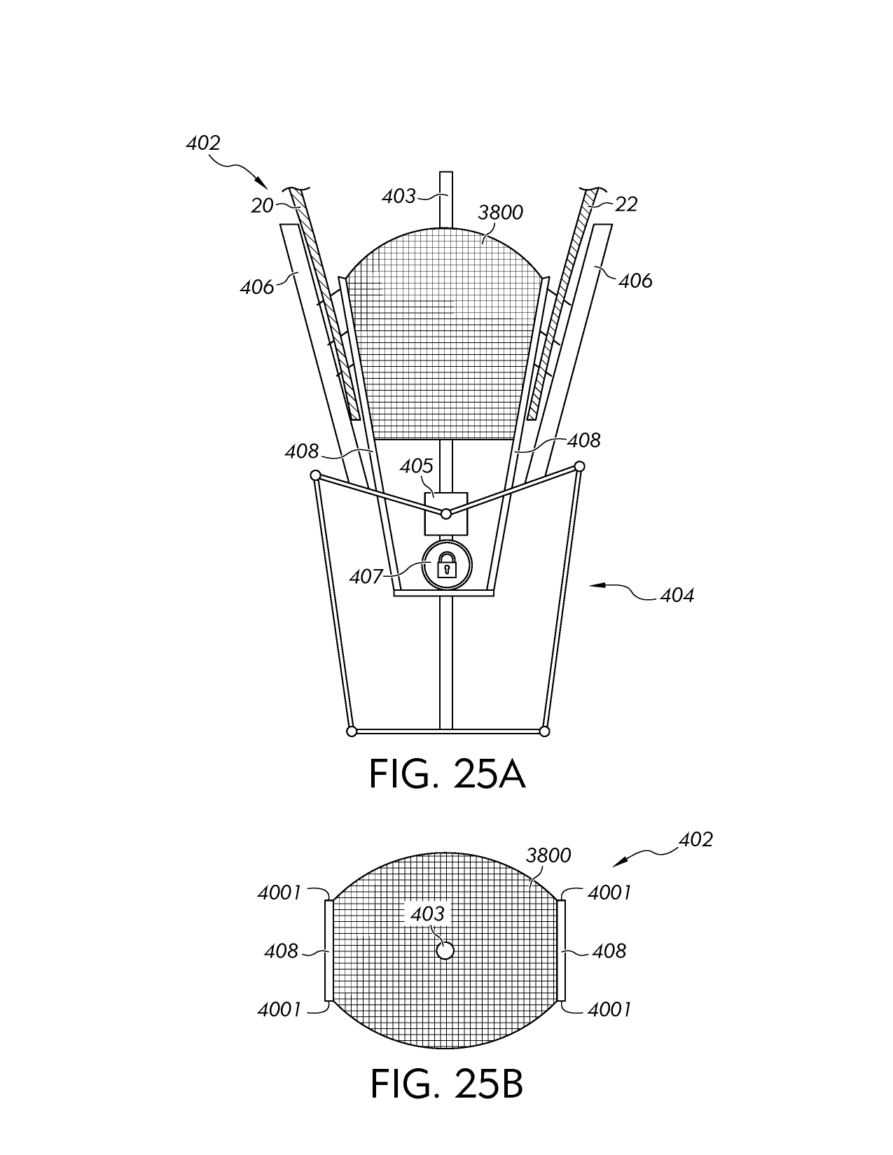

Referring to FIGS. 25A-25B, an example implementation of a treatment and/or repair device 402 has a coaptation element 3800. The device 402 can have the same configuration as the device illustrated by FIG. 24 with the addition of the coaptation element. The coaptation element 3800 can take a wide variety of different forms. The coaptation element 3800 can be compressible and/or expandable. For example, the coaptation element can be compressed to fit inside one or more catheters of a delivery system, can expand when moved out of the one or more catheters, and/or can be compressed by the paddles 406 to adjust the size of the coaptation element. In the example illustrated by FIGS. 25A and 25B, the size of the coaptation element 3800 can be reduced by squeezing the coaptation element with the paddles 406 and can be increased by moving the paddles 406 away from one another. The coaptation element 3800 can extend past outer edges 4001 of the gripping members or clasps 408 as illustrated for providing additional surface area for closing the gap of a mitral valve.

The coaptation element 3800 can be coupled to the device 402 in a variety of different ways. For example, the coaptation element 3800 can be fixed to the shaft 403, can be slidably disposed around the shaft, can be connected to the coupler 405, can be connected to the lock 407, and/or can be connected to a central portion of the clasps or gripping members 408. In some implementations, the coupler 405 can take the form of the coaptation element 3800. That is, a single element can be used as the coupler 405 that causes the paddles 406 to move between the open and closed positions and the coaptation element 3800 that closes the gap between the leaflets 20, 22 when the device 402 is attached to the leaflets.

The coaptation element 3800 can be disposed around one or more of the shafts or other control elements of the system 400. For example, the coaptation element 3800 can be disposed around the shaft 403, the shaft 413, the paddle control mechanism 410, and/or the lock control mechanism 412.

The device 402 can include any other features for a device, treatment device, repair device, implant, etc. discussed in the present application, and the device 402 can be positioned to engage valve tissue as part of any suitable treatment and/or repair system (e.g., any valve repair system and/or treatment system disclosed in the present application). Additional features of the device 402, modified versions of the device, delivery systems for the device, and methods for using the device and delivery system are disclosed by Patent Cooperation Treaty International Application No. PCT/US2019/012707 (International Publication No. WO 2019139904). Any combination or sub-combination of the features disclosed by the present application can be combined with any combination or sub-combination of the features disclosed by Patent Cooperation Treaty International Application No. PCT/US2019/012707 (International Publication No. WO 2019139904).

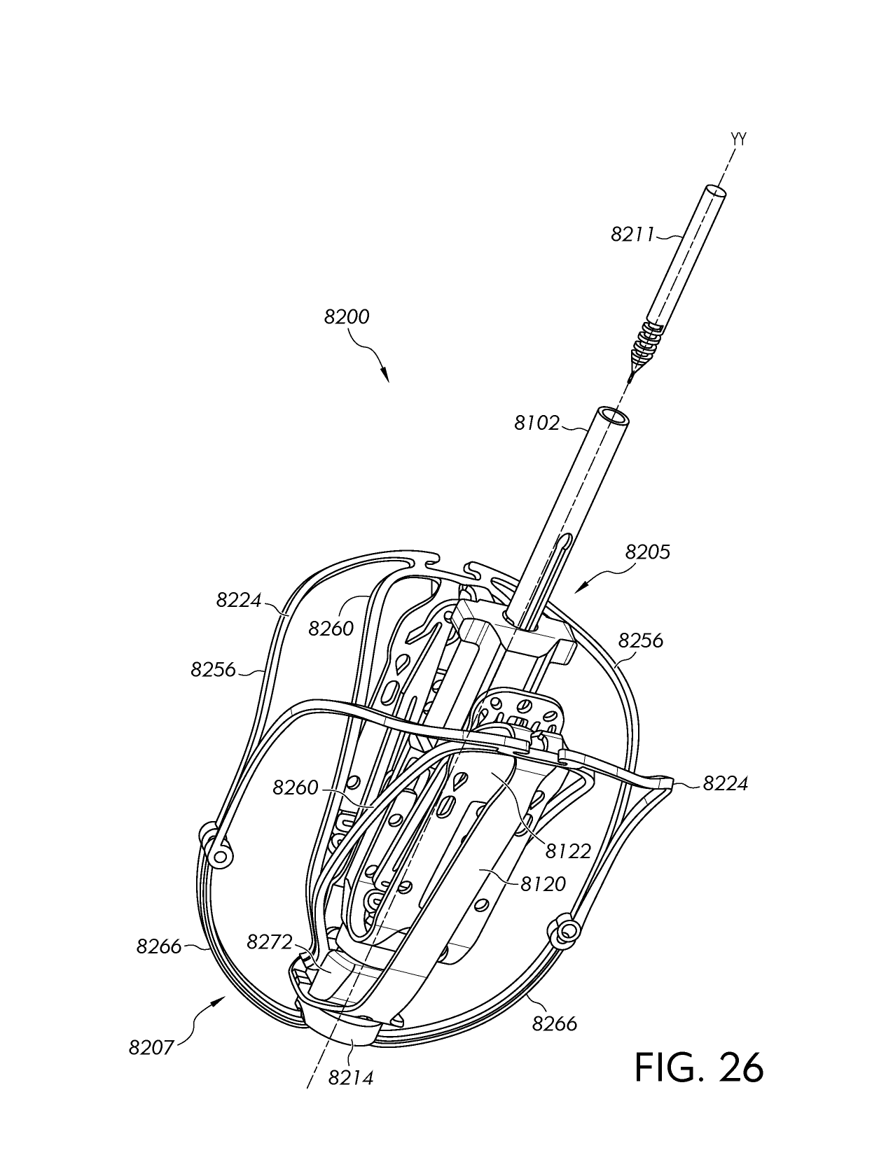

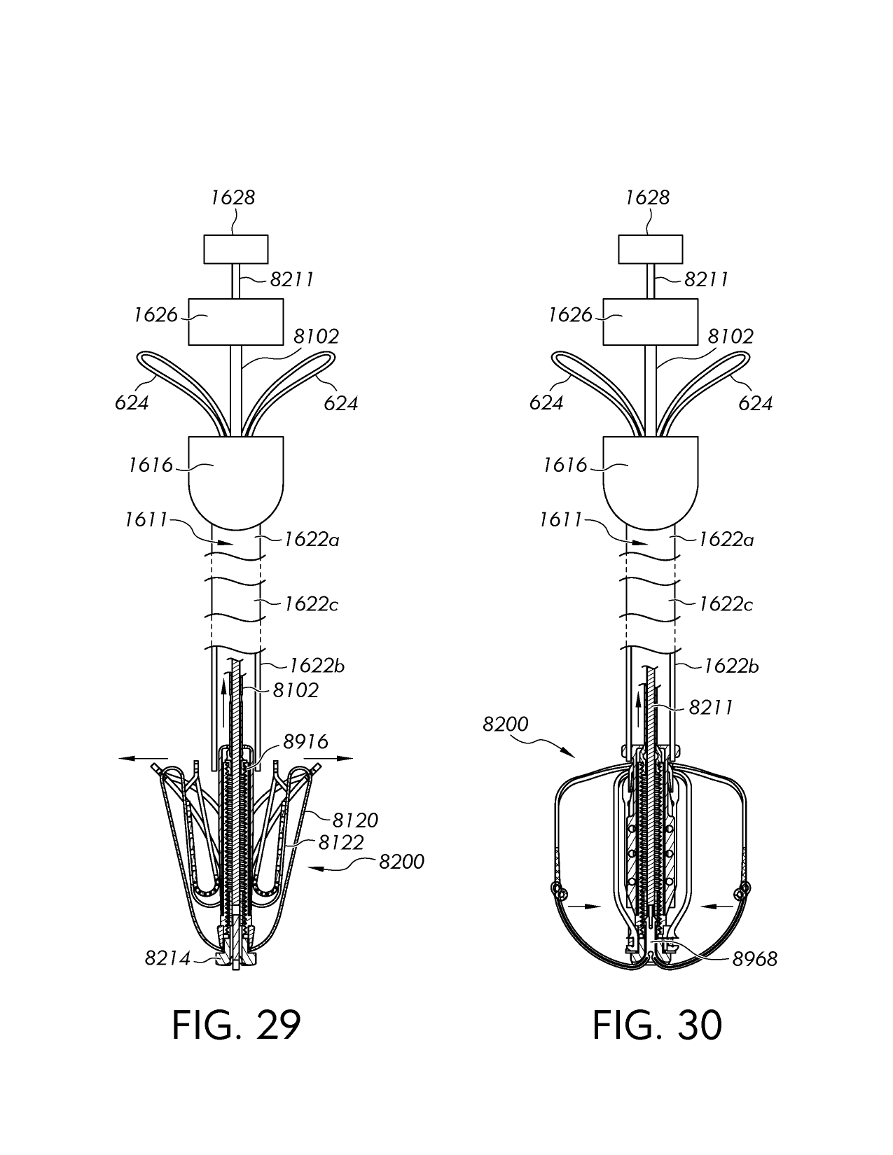

FIGS. 26-30 illustrate an example of one of the many systems for treating and/or repairing a native valve of a patient that the concepts of the present application can be applied to. Referring to FIGS. 29 and 30, the system includes a catheter assembly 1611 (e.g., a device catheter assembly, an implant catheter assembly, treatment catheter assembly, etc.) and a treatment and/or repair device 8200. Referring to FIGS. 26-28, the device 8200 includes a proximal or attachment portion 8205, paddle frames 8224, and a distal portion 8207. The attachment portion 8205, the distal portion 8207, and the paddle frames 8224 can be configured in a variety of ways.

In the example illustrated in FIG. 26, the paddle frames 8224 can be symmetric along longitudinal axis YY. However, in some implementations, the paddle frames 8224 are not symmetric about the axis YY. Moreover, referring to FIG. 26, the paddle frames 8224 include outer frame portions 8256 and inner frame portions 8260.

In some implementations, the connector 8266 (e.g., shaped metal component, shaped plastic component, tether, wire, strut, line, cord, suture, etc.) attaches to the outer frame portions 8256 at outer ends of the connector 8266 and to a coupler 8972 at an inner end 8968 of the connector 8266 (see FIG. 28). Between the connector 8266 and the attachment portion 8205, the outer frame portions 8256 form a curved shape. For example, in the illustrated example, the shape of the outer frame portions 8256 resembles an apple shape in which the outer frame portions 8256 are wider toward the attachment portion 8205 and narrower toward the distal portion 8207. In some implementations, however, the outer frame portions 8256 can be otherwise shaped.

The inner frame portions 8260 extend from the attachment portion 8205 toward the distal portion 8207. The inner frame portions 8260 then extend inward to form retaining portions 8272 that are attached to the actuation cap 8214. The retaining portions 8272 and the actuation cap 8214 can be configured to attach in any suitable manner.

In some implementations, the inner frame portions 8260 are rigid frame portions, while the outer frame portions 8256 are flexible frame portions. The proximal end of the outer frame portions 8256 connect to the proximal end of the inner frame portions 8260, as illustrated in FIG. 26.

The width adjustment element 8211 (e.g., width adjustment wire, width adjustment shaft, width adjustment tube, width adjustment line, width adjustment cord, width adjustment suture, width adjustment screw or bolt, etc.) is configured to move the outer frame portions 8256 from the expanded position to the narrowed position by pulling the inner end 8968 (FIG. 28) and portions of the connector 8266 into the actuation cap 8214. The actuation element 8102 is configured to move the inner frame portions 8260 to open and close the inner and outer paddle portions 8122, 8120 in accordance with some implementations disclosed herein.

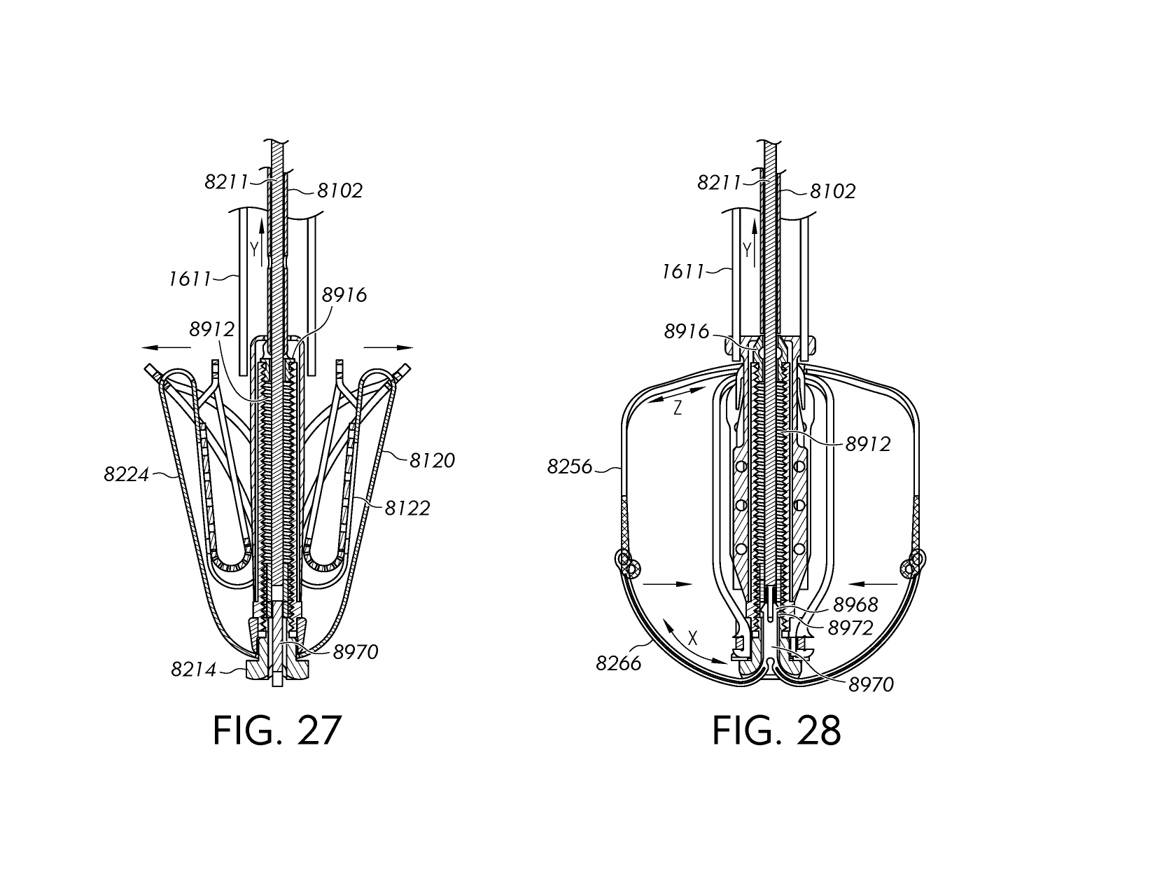

As shown in FIGS. 27 and 28, the connector 8266 has an inner end 8968 that engages with the width adjustment element 8211 such that a user can move the inner end 8968 inside the receiver 8912 (e.g., an internally threaded element, a column, a conduit, a hollow member, a notched receiving portion, a tube, a shaft, a sleeve, a post, a housing, a cylinder, tracks, etc.) to move the outer frame portions 8256 between a narrowed position and an expanded position. In the illustrated example, the inner end 8968 includes a post 8970 that attaches to the outer frame portions 8256 and a coupler 8972 that extends from the post 8970. The coupler 8972 is configured to attach and detach from both the width adjustment element 8211 and the receiver 8912. The coupler 8972 can take a wide variety of different forms. For example, the coupler 8972 can include one or more of a threaded connection, features that mate with threads, detent connections, such as outwardly biased arms, walls or other portions. When the coupler 8972 is attached to the width adjustment element 8211, the coupler is released from the receiver 8912. When the coupler 8972 is detached from the width adjustment element 8211, the coupler is secured to the receiver. The inner end 8968 of the connector can, however, be configured in a variety of ways. Any configuration that can suitably attach the outer frame portions 8256 to the coupler to allow the width adjustment element 8211 to move the outer frame portions 8256 between the narrowed position and the expanded position can be used. The coupler can be configured in a variety of ways as well and can be a separate component or be integral with another portion of the device, e.g., of the connector or inner end of the connector.

The width adjustment element 8211 allows a user to expand or contract the outer frame portions 8256 of the device 8200. In the example illustrated in FIGS. 27 and 28, the width adjustment element 8211 includes an externally threaded end that is threaded into the coupler 8972. The width adjustment element 8211 moves the coupler in the receiver 8912 to adjust the width of the outer frame portions 8256. When the width adjustment element 8211 is unscrewed from the coupler 8972, the coupler engages the inner surface of the receiver 8912 to set the width of the outer frame portions 8256.

In some implementations, the receiver 8912 can be integrally formed with a distal cap 8214. Moving the cap 8214 relative to a body of the attachment portion 8205 opens and closes the paddles. In the illustrated example, the receiver 8912 slides inside the body of the attachment portion. When the coupler 8972 is detached from the width adjustment element 8211, the width of the outer frame portions 8256 is fixed while the actuation element 8102 moves the receiver 8912 and cap 8214 relative to a body of the attachment portion 8205. Movement of the cap can open and close the device in the same manner as some of the examples disclosed above.

In the illustrated example, a driver head 8916 is disposed at a proximal end of the actuation element 8102. The driver head 8916 releasably couples the actuation element 8102 to the receiver 8912. In the illustrated example, the width adjustment element 8211 extends through the actuation element 8102. The actuation element is axially advanced in the direction opposite to direction Y to move the distal cap 8214. Movement of the distal cap 8214 relative to the attachment portion 8205 is effective to open and close the paddles, as indicated by the arrows in FIG. 27. That is, movement of the distal cap 8214 in the direction Y closes the device and movement of the distal cap in the direction opposite to direction Y opens the device.

Also illustrated in FIGS. 27 and 28, the width adjustment element 8211 extends through the actuation element 8102, the driver head 8916, and the receiver 8912 to engage the coupler 8972 attached to the inner end 8968. The movement of the outer frame portions 8256 to the narrowed position can allow the device or implant 8200 to maneuver more easily into position for deployment and/or implantation in the heart by reducing the contact and/or friction between the native structures of the heart—e.g., chordae—and the device 8200. The movement of the outer frame portions 8256 to the expanded position provides the anchor portion of the device 8200 with a larger surface area to engage and capture leaflet(s) of a native heart valve.

Referring to FIGS. 29 and 30, an example of a catheter assembly 1611 (e.g., a device catheter assembly, an implant catheter assembly, treatment catheter assembly, etc.) in which clasp actuation lines 624 extend through a handle 1616, the actuation element 8102 is coupled to a paddle actuation control 1626, and the width adjustment element 8211 is coupled to a paddle width control 1628. A proximal end portion 1622a of the shaft or catheter of the catheter assembly 1611 can be coupled to the handle 1616, and a distal end portion 1622b of the shaft or catheter can be coupled to the device 8200. The actuation element 8102 can extend distally from the paddle actuation control 1626, through the handle 1616, through the delivery shaft or catheter of the catheter assembly 1611, and through the proximal end of the device 8200, where it couples with the driver head 8916. The actuation element 8102 can be axially movable relative to the outer shaft of the catheter assembly 1611 and the handle 1616 to open and close the device.

The width adjustment element 8211 can extend distally from the paddle width control 1628, through the paddle actuation control 1626 and through the actuation element 8102 (and, consequently, through the handle 1616, the outer shaft of the implant catheter assembly 1611, and through the device 8200), where it couples with the movable coupler 8972. The width adjustment element 8211 can be axially movable relative to the actuation element 8102, the outer shaft of the implant catheter assembly 1611, and the handle 1616. The clasp actuation lines 624 can extend through and be axially movable relative to the handle 1616 and the outer shaft of the implant catheter assembly 1611. The clasp actuation lines 624 can also be axially movable relative to the actuation element 8102.

Referring to FIGS. 29 and 30, the width adjustment element 8211 can be releasably coupled to the coupler 8972 of the device 8200. Advancing and retracting the width adjustment element 8211 with the paddle width control 1628 widens and narrows the paddles. Advancing and retracting the actuation element 8102 with the paddle actuation control 1626 opens and closes the paddles of the device.

In the examples of FIGS. 29 and 30, the catheter or shaft of the catheter assembly 1611 is an elongate shaft extending axially between the proximal end portion 1622a, which is coupled to the handle 1616, and the distal end portion 1622b, which is coupled to the device 8200. The outer shaft of the catheter assembly 1611 can also include an intermediate portion 1622c disposed between the proximal and distal end portions 1622a, 1622b.

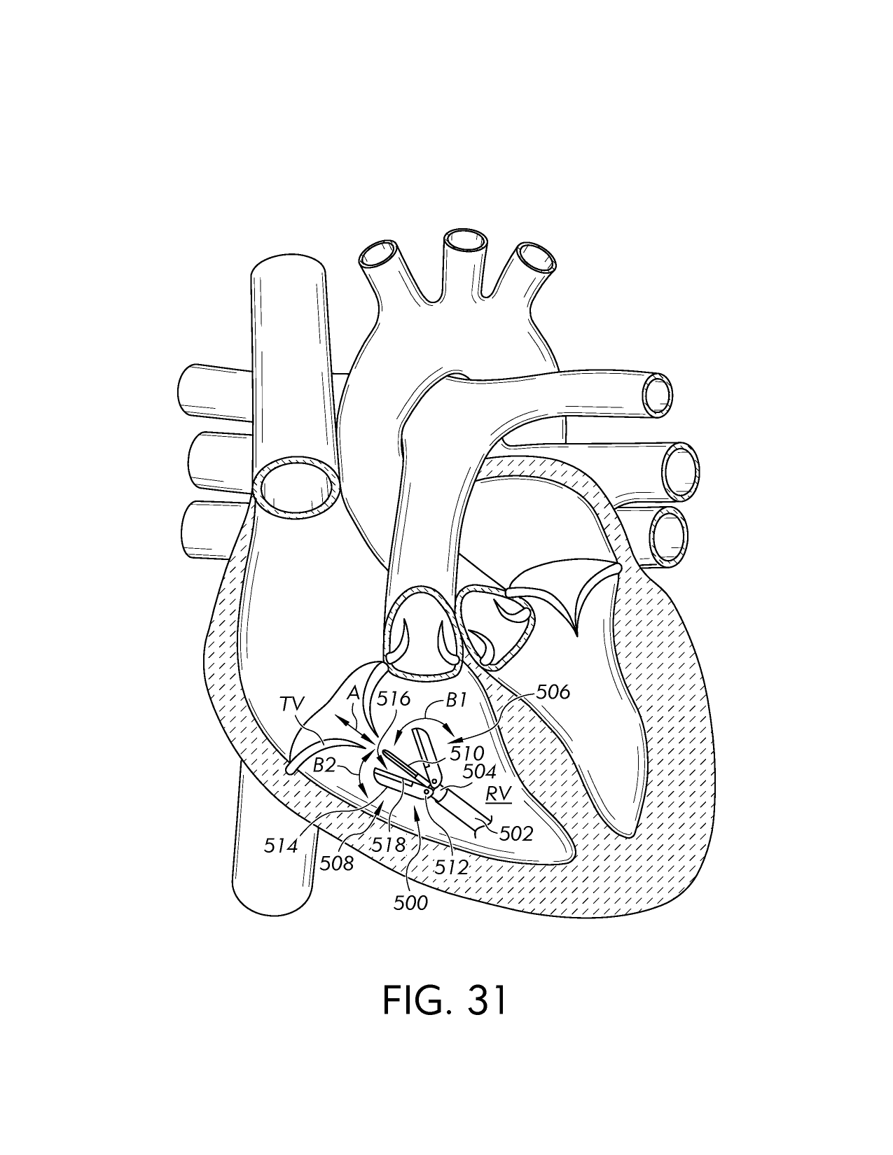

Referring to FIGS. 31-33, an example leaflet attachment device 500 is illustrated. The leaflet attachment device 500 is configured to be delivered to the location of a native heart valve (e.g., mitral valve, tricuspid valve). The leaflet attachment device 500 can be configured to be delivered via a delivery system 502 or other means for delivery. The delivery system 502 can be any suitable delivery system. For example, the delivery system 502 can be the same as, or similar to, the delivery system 102 described above and can comprise one or more of a catheter, a sheath, a guide catheter/sheath, a delivery catheter/sheath, a steerable catheter, an implant catheter, a tube, a channel, a pathway, combinations of these, etc. In some implementations, the device 500 is disposed at the end of a catheter of the delivery system 502.

Once delivered into position, the leaflet attachment device 500 is configured to engage leaflets (e.g., leaflets 20, 22, 30, 32, 34) of a native heart valve to attach the leaflets together (i.e., directly or indirectly). In some implementations, the leaflet attachment device 500 is configured to capture a first leaflet (e.g., the anterior leaflet 30 of the tricuspid valve TV or the anterior leaflet 20 of the mitral valve) and a second leaflet (e.g., the septal leaflet 32 of the tricuspid valve TV or the posterior leaflet 22 of the mitral valve) of a native heart valve. Once captured, the leaflet attachment device 500 is configured to attach (e.g., by tissue fusion, an adhesive, stitches, and/or anchors, etc.) the first leaflet and the second leaflet together, either directly to one another and/or to an intermediate structure.

In some implementations, the leaflet attachment device 500 can be configured to fuse the leaflets together. For example, bipolar (radiofrequency) and/or ultrasonic energy can be used to fuse collagen and elastin in bodily tissues. The leaflet attachment device 500 can be configured to output energy at the leaflets to fuse the leaflets together.

The leaflet attachment device 500 can be configured to grasp and fuse the leaflets in a variety of ways. In the illustrated example, the leaflet attachment device 500 includes a base 504, a first surface of a first arm 506, a second surface of a second arm 508, and a center element 510 (e.g., rod, tube, post, beam, pipe, column, needle, etc.). While first arm 506 and second arm 508 and other arms herein are used by way of example, surfaces on other components can be used in similar ways, even if not on an arm per se.

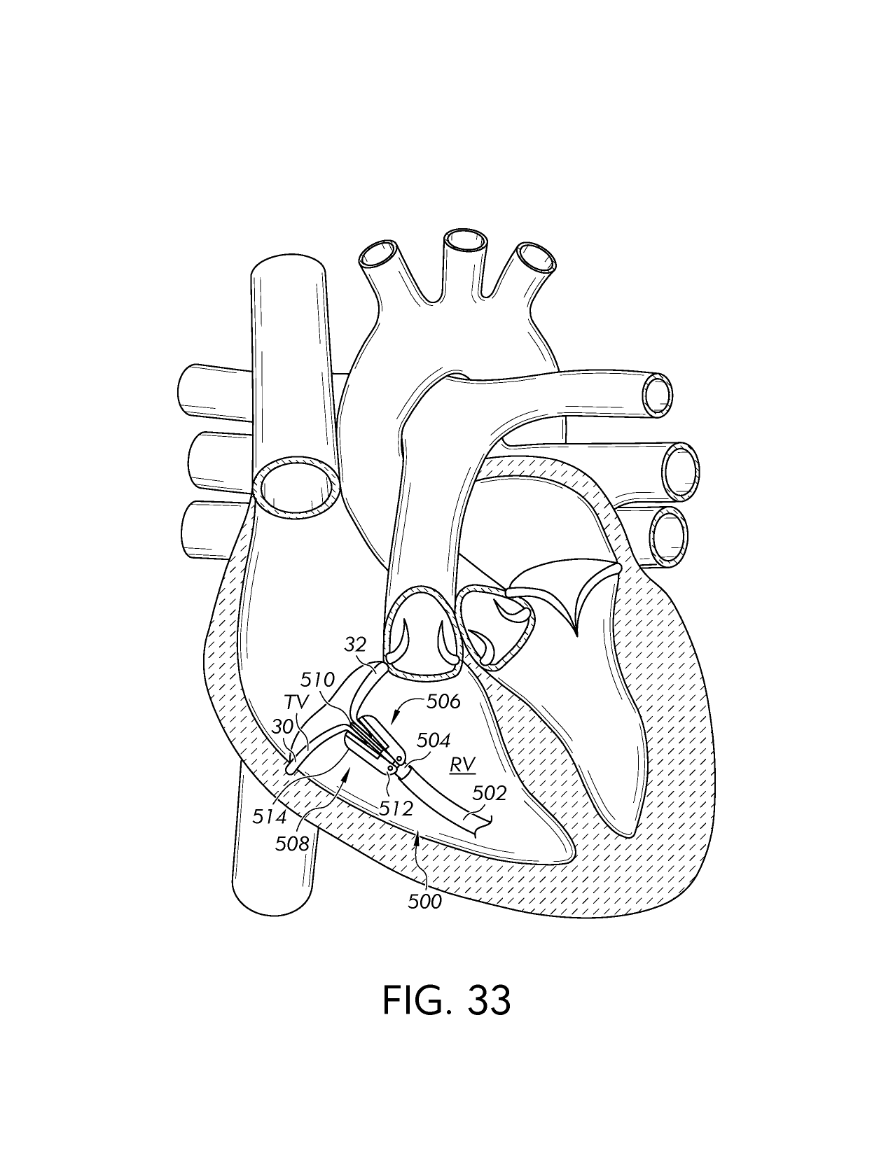

In some implementations, the first arm 506 and the second arm 508 are configured to grasp leaflets of the heart valve (e.g., between surfaces thereof). In the illustrated example, the first arm 506 and the second arm 508 are pivotable relative to the base 504 between an open condition (FIG. 31) and a closed condition (FIG. 33).

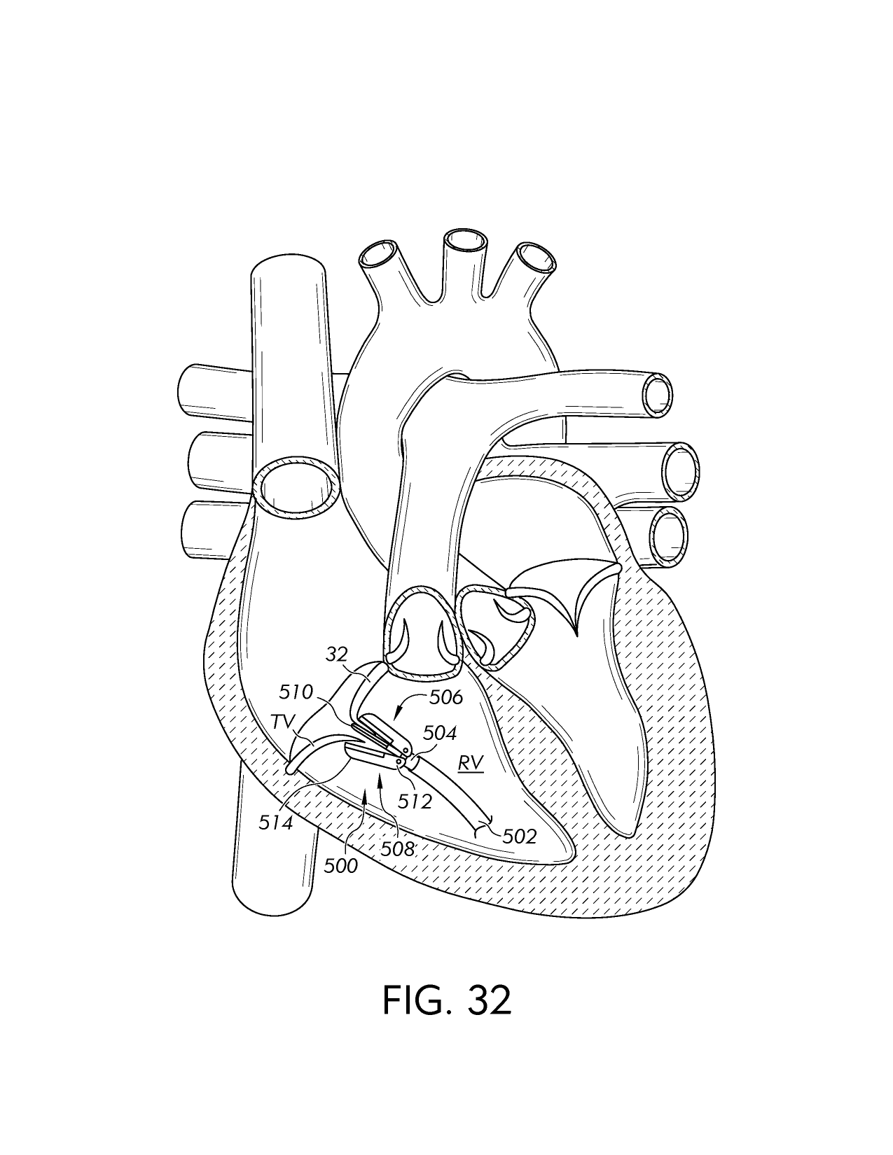

In some implementations, the first arm 506 and the second arm 508 are movable independent of each other, as shown in FIG. 32. The first arm 506 and the second arm 508 can be moved between the open condition and the closed condition by any suitable means. For example, an actuation element (not shown) (e.g., actuation wire, shaft, tube, hypotube, line, suture, braid, etc.), such as any actuation element disclosed herein, can be provided to move the first arm 506 and the second arm 508 between the open condition and the closed condition.

Each of the first arm 506 and the second arm 508 can be configured in a variety of ways. In some implementations, the first arm 506 and the second arm 508 are configured the same or similar to each other. In the illustrated example, each of the first arm 506 and the second arm 508 has a generally elongated body that includes a proximal end portion 512, a distal end portion 514, and an inner face 516 (FIG. 31) extending between the proximal end portion 512 and the distal end portion 514. The proximal end portion 512 can be attached to the base 504 in any suitable manner that allows the first arm 506 and the second arm 508 to pivot or otherwise move relative to the base 504.

In some implementations, the proximal end portion 512 is attached to the base 504 by a pivot connection (e.g., pivot pin). In some implementations, the proximal end portion 512 is fixedly attached to the base 504 and includes a flexible or bendable portion that allow the first arm 506 and the second arm 508 to bend or flex at the flexible or bendable portion for the first arm 506 and the second arm 508 to pivot relative to the base 504.

In some implementations, the inner face 516 of each arm 506, 508 includes a contact surface 518 (e.g., pad, ridge, projection, block, or other suitable surface) configured to contact a leaflet when the leaflet is grasped.

In some implementations, the contact surface 518 is configured to output energy (e.g., heat up in response to radiofrequency, ultrasonic, or other suitable energy) to fuse the leaflets together. In some implementations, as shown in FIGS. 31-33, the contact surface 518 can be a continuous surface that extends along a portion of the inner surface 516. In other implementations, the contact surface can be discontinuous (e.g., a plurality of discrete surfaces on the inner face 516) or can extend the entire length of the inner face 516.

In some implementations, the center element 510 is configured to extend between the first arm 506 and the second arm 508. The center element 510 allows each arm 506, 508 to capture a leaflet against the center element 510 independently.

In some implementations, the center element 510 can be a dynamic member that can be retracted (i.e., moved out from between the captured leaflets) or have a geometry that allows more direct compression between the contact surfaces 518 when the leaflets are captured.

FIG. 31 illustrates the leaflet attachment device 500 positioned in the right ventricle RV with both the first arm 506 and the second arm 508 in the open condition to engage the tricuspid valve TV. The device 500, however, can be delivered to other locations in the heart and/or can engage other valves (e.g., the mitral valve). In FIG. 31., arrow A illustrates that the leaflet attachment device 500 can be advanced into the tricuspid valve TV or retracted away from the tricuspid valve TV as needed in order to suitably position the leaflet attachment device 500 to capture the leaflets. Arrows B1, B2 illustrate that the first and second arms 506, 508 can pivot inward to the closed condition and outward to the open condition as needed.

Once the leaflet attachment device 500 is properly positioned, the first and second surfaces and/or arms 506, 508 can be moved to the closed condition to capture leaflets. For example, FIG. 32 illustrates the first arm 506 in the closed condition with the septal leaflet 32 captured between the first arm 506 and the center element 510. With the septal leaflet 32 captured, the leaflet attachment device 500 can be repositioned (within the constraints of being attached to the septal leaflet 32) to capture another of the tricuspid valve leaflets 30, 34. In the example of FIG. 33, the second arm 508 is illustrated in the closed condition with the anterior leaflet 30 captured between the second arm 508 and the center element 510.

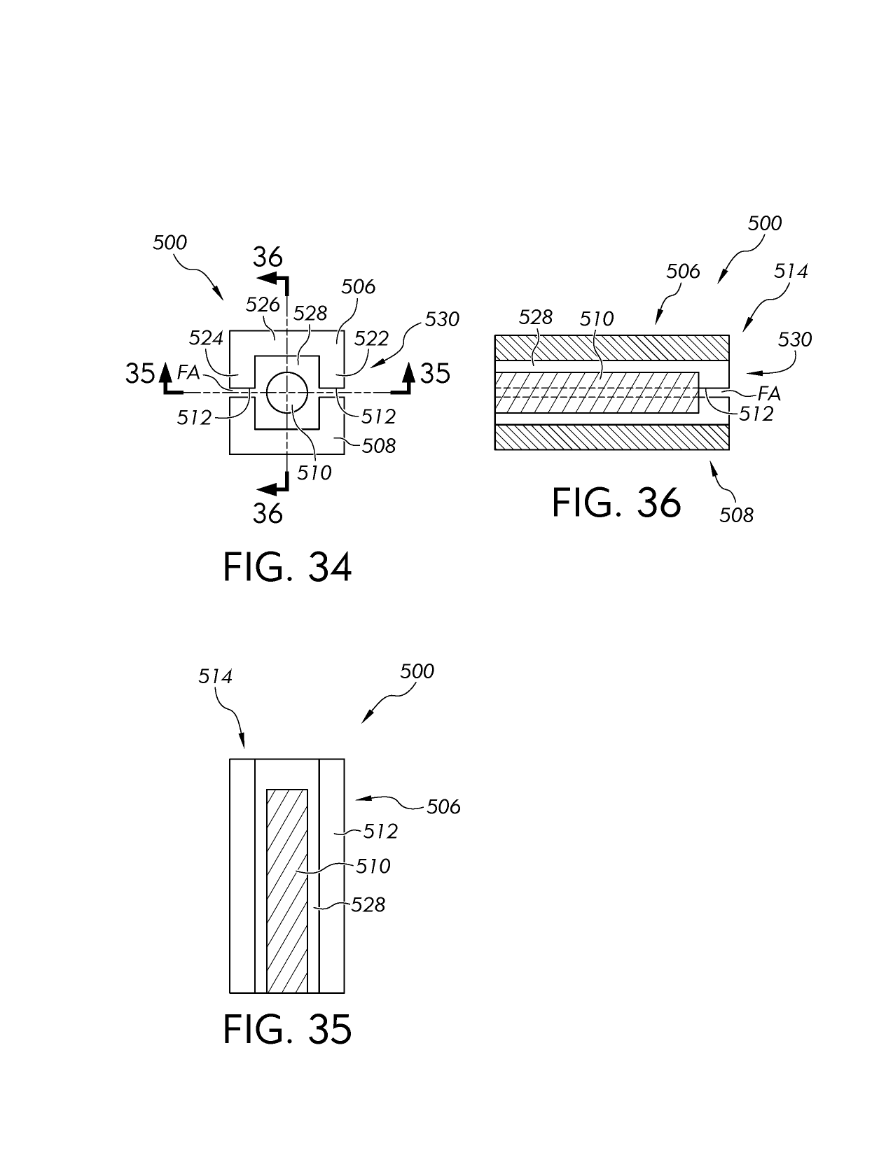

In some implementations, with the leaflets 30, 32 captured, the leaflet attachment device 500 can fuse the leaflets together by energizing the contact surfaces 518. The contact surfaces 518 can be configured in a variety of ways. FIGS. 34-36 illustrate an example configuration of the contact surfaces 518 and first and second arms 506, 508. In particular, each of the first and second arms 506, 508 have an inner surface 516, or portion thereof, that is generally U-shaped (FIG. 34). For example, the first arm 506 includes a first projecting portion 522, a second projecting portion 524 spaced apart from the first projecting portion 522, and an intermediate portion 526 extending between and connecting the first and the second projecting portion 522, 524 such that a channel 528 is formed therebetween. The contact surfaces 512 are positioned at the distal end 530 of each of the first and the second projecting portion 522, 524.

In the illustrated examples, the second arm 508 is configured the same as, or like, the first arm 506 and arranged in a mirror image to the first arm 506 as shown in FIG. 34. When the first arm 506 is in the closed condition, the captured first leaflet is pinched between the first arm 506 and the center element 510. Likewise, when the second arm 508 is in the closed condition, the captured second leaflet is pinched between the second arm 508 and the center element 510. With both the first and the second arms 506, 508 in the closed condition, the first and second leaflets are also pinched between the contact surfaces 512 on the projecting portions 522, 524 of the first and the second arms 506, 508.

In some implementations, once the first and second leaflets are pinched between the contact surfaces 512 of the first and the second arms 506, 508, the leaflet attachment device 500 can be energized such that the contact surfaces 512 fuse the leaflets together in the area FA between the contact surfaces 512.

In some implementations, the center element 510 is retractable and can be removed prior to fusing the leaflets together.

In some implementations, the center element 510 is still in place between the first and the second arms 506, 508 when the leaflets are fused together.

In some implementations, where the leaflets are fused together on either side of the center element 510, or where the center element 510 was prior to being retracted, there can be an unfused section between the leaflets that is between two fused sections. In some implementations, the unfused section can provide an opening in which the leaflet attachment device 500 can be withdrawn if needed, depending on the direction of deployment of the leaflet attachment device.

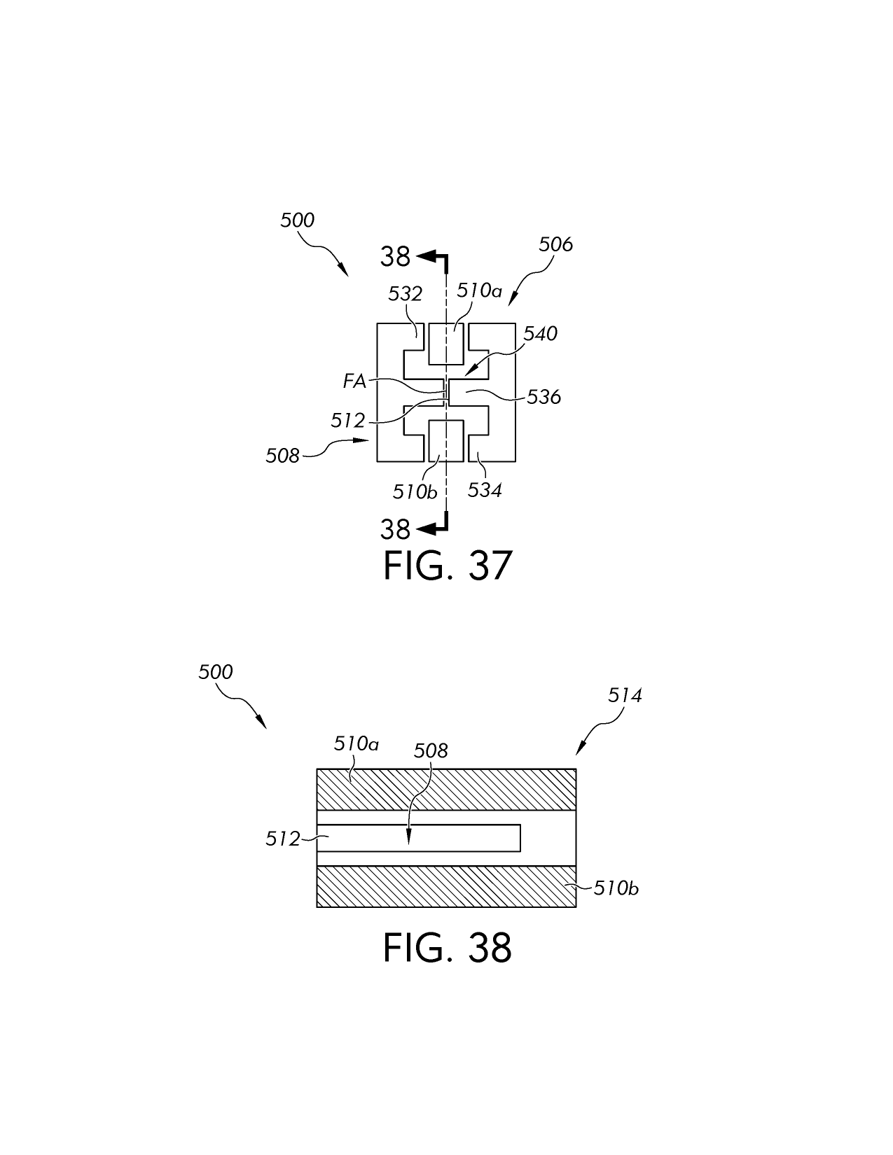

FIGS. 37-38 illustrate an example configuration of the contact surfaces 518 in which a single fused section is formed. In the example of FIGS. 37-38, the device 500 includes a first center member or portion 510a and a second center member or portion 510b. In some implementations, the first center member or portion 510a and the second center member or portion 510b are two independent coaptation elements. In some implementations, the first center member or portion 510a and a second center member or portion 510b are formed as a bifurcated distal end 514 of a single coaptation element.

In some implementations, each of the first and second arms 506, 508 have an inner surface 516, or portion thereof, that is generally W-shaped (FIG. 37). For example, the first arm 506 can include a first projecting portion 532, a second projecting portion 534 spaced apart from the first projecting portion 522, and a third projecting portion 536 between and connecting the first and the second projecting portion 532, 534. As shown in FIG. 37, in some implementations, the third projecting portion 536 is longer than the first and the second projecting portions 532, 534. In some implementations, the contact surface 512 is positioned at the distal end 540 of the third projecting portion 536.

In the illustrated example, the second arm 508 is configured the same as, or like, the first arm 506 and arranged in a mirror image to the first arm 506 as shown in FIG. 37. In some implementations, when the first arm 506 is in the closed condition, the captured first leaflet is pinched between the first projecting portion 532 and first center element 510a and between the second projecting portion 534 and the second center element 510b. Likewise, when the second arm 508 is in the closed condition, the captured second leaflet is pinched between the first projecting portion 532 and first center element 510a and between the second projecting portion 534 and the second center element 510b on the second arm. With both the first and the second arms 506, 508 in the closed condition, the first and second leaflets are also pinched between the contact surfaces 512 on the third projecting portions 536 of the first and the second arms 506, 508.

In some implementations, once the first and second leaflets are pinched between the contact surfaces 512 of the first and the second arms 506, 508, the leaflet attachment device 500 can be energized such that the contact surfaces 512 fuse the leaflets together in the area FA between the contact surfaces 512. In some implementations, the coaptation elements 510a, 510b are still in place between the first and the second arms 506, 508 when the leaflets are fused together. In other implementations, the center elements 510a, 510b are retractable and can be removed prior to fusing the leaflets together.

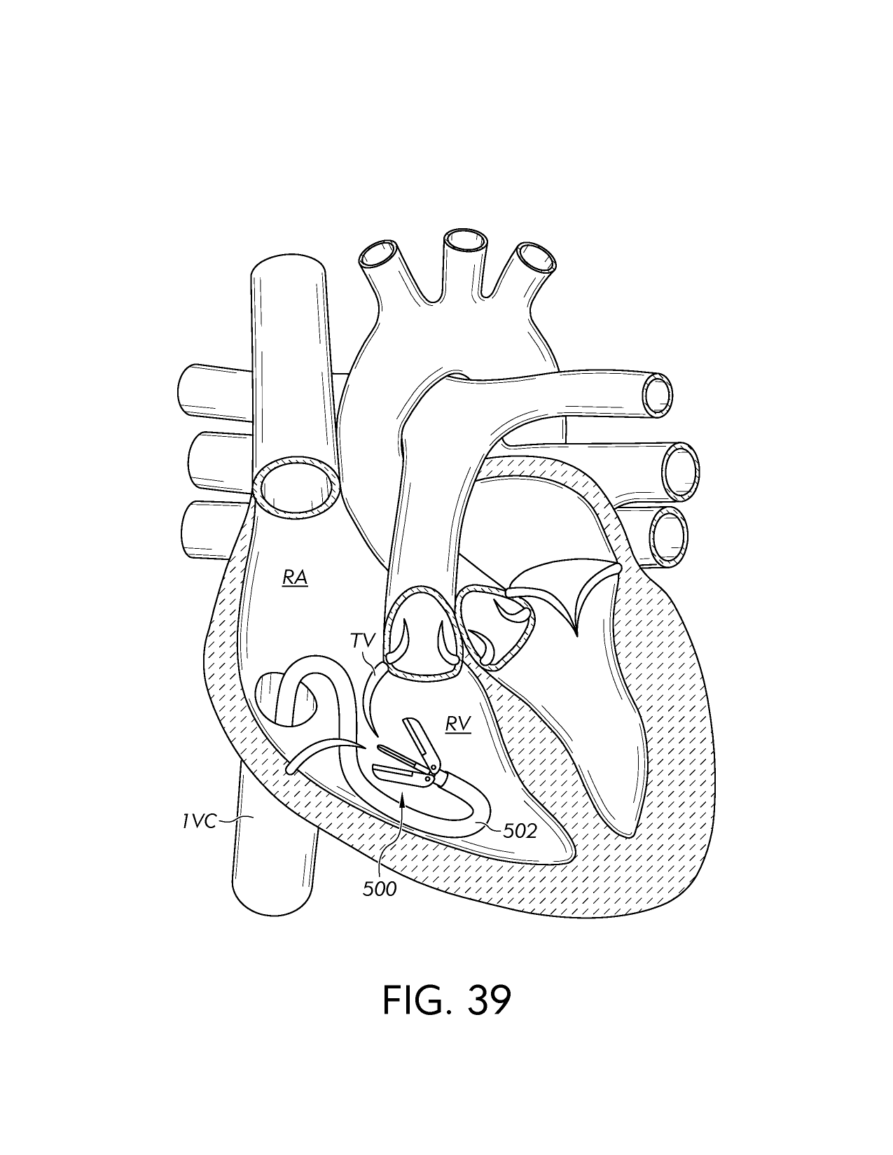

FIG. 39 illustrates an example method of delivering the tissue attachment device 500 to the tricuspid valve TV. In the example of FIG. 39, the tissue attachment device 500 is disposed at the end of a catheter of the delivery system 502. While the tissue attachment device 500 is illustrated in the open condition in preparation to capture leaflets of the tricuspid valve TV, during delivery the tissue attachment device 500 is preferably in the closed condition to more easily be navigated into position.

As shown in FIG. 39, the tissue attachment device 500 can be delivered to the tricuspid valve TV from the interior vena cava IVC. In particular, the catheter of the delivery system 502 can be extended through the interior vena cava IVC and into the right atrium RA. From the right atrium RA, the catheter can transport the tissue attachment device 500 through the tricuspid valve TV and into the right ventricle RV. While in the right ventricle RV, the catheter can be steered to position the tissue attachment device 500 below the tricuspid valve TV in position to capture the leaflets of the tricuspid valve TV, as shown in FIG. 39. For example, if the tissue attachment device 500 is used to fuse the anterior leaflet 30 and the septal leaflet 32 together, the catheter can extend through the tricuspid valve TV via the area of the posterior leaflet 34 of the tricuspid valve TV or between the anterior leaflet 30 and the septal leaflet 32 in an area not to be fused (e.g., at the commissure).

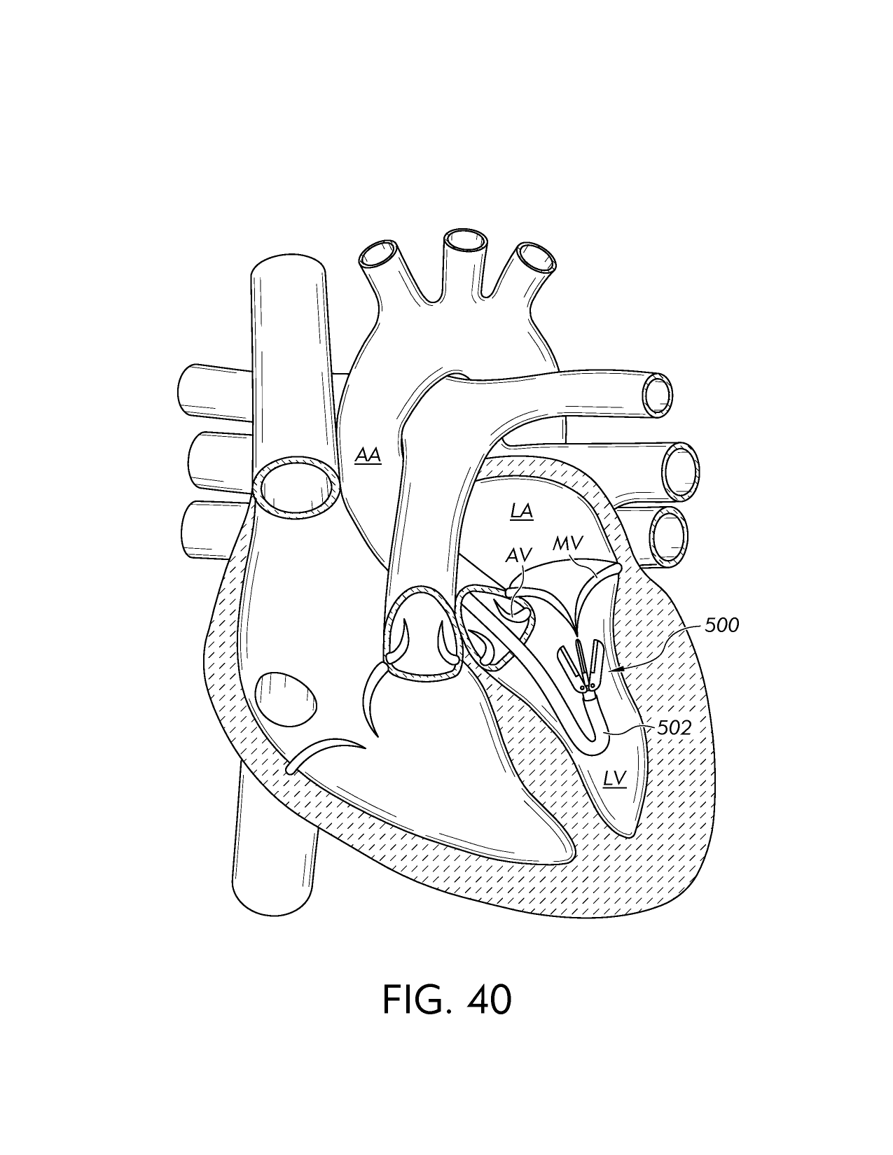

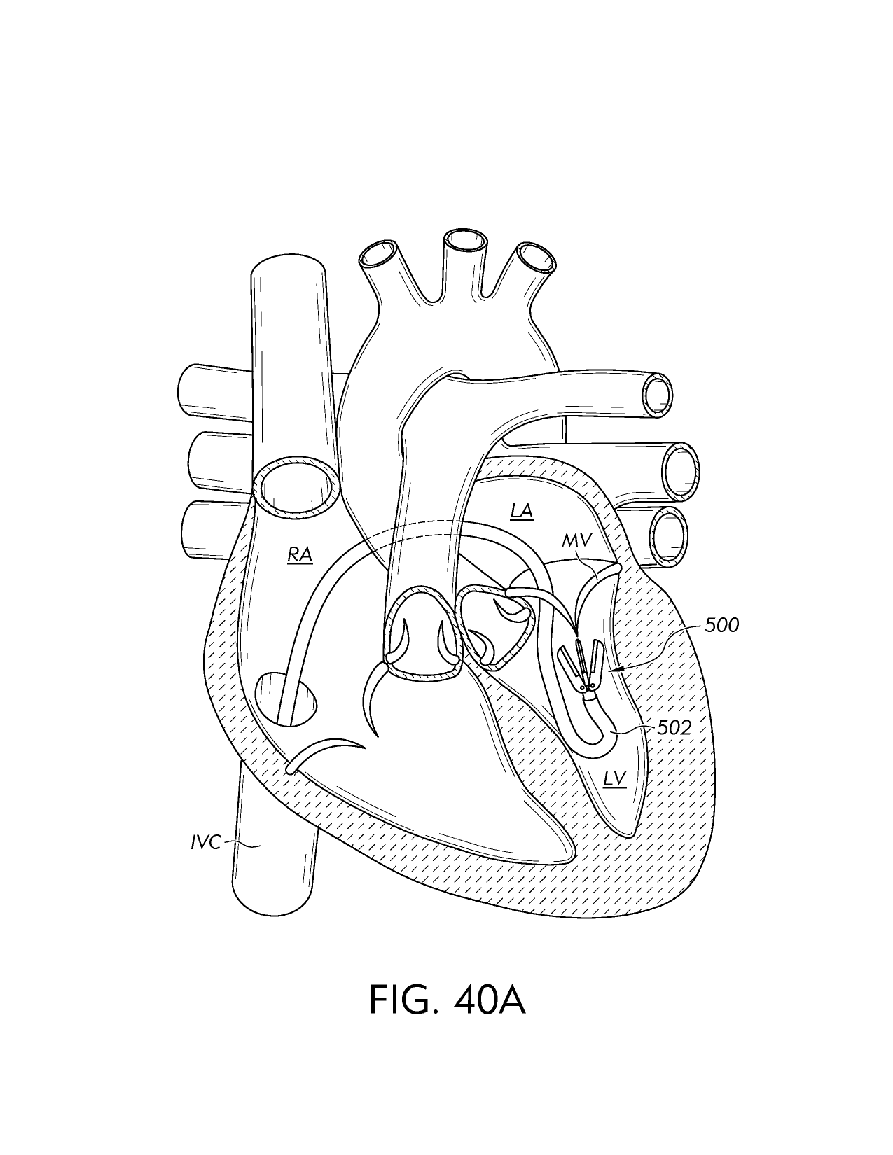

FIG. 40 illustrates an example method of delivering the tissue attachment device 500 to the mitral valve MV. In the example of FIG. 40, the tissue attachment device 500 is disposed at the end of a catheter of the delivery system 502. While the tissue attachment device 500 is illustrated in the open condition in preparation to capture leaflets of the tricuspid valve TV, during delivery the tissue attachment device 500 is preferably in the closed condition to more easily be navigated into position.

As shown in FIG. 40, the tissue attachment device 500 can be delivered to the mitral valve MV from the ascending aorta AA. In particular, the catheter of the delivery system 502 can be extended through the aortic valve AV and into the left ventricle LV. While in the left ventricle LV, the catheter can be steered to position the tissue attachment device 500 below the mitral valve MV in position to capture the leaflets of the mitral valve MV, as shown in FIG. 40.

FIG. 40A illustrates an example method of delivering the tissue attachment device 500 to the mitral valve MV. In the example of FIG. 40A, the tissue attachment device 500 is disposed at the end of a catheter of the delivery system 502. While the tissue attachment device 500 is illustrated in the open condition in preparation to capture leaflets of the tricuspid valve TV, during delivery the tissue attachment device 500 is preferably in the closed condition to more easily be navigated into position.

As shown in FIG. 40A, the tissue attachment device 500 can be delivered into the right atrium RA via the interior vena cava IVC and through the atrial septum into the left atrium LA. From the left atrium LA, the catheter can extend through the mitral valve MV and into the left ventricle LV. While in the left ventricle LV, the catheter can be steered to position the tissue attachment device 500 below the mitral valve MV in position to capture the leaflets of the mitral valve MV, as shown in FIG. 40A. The catheter can extend through the mitral valve MV in an area not to be fused (e.g., the commissure).

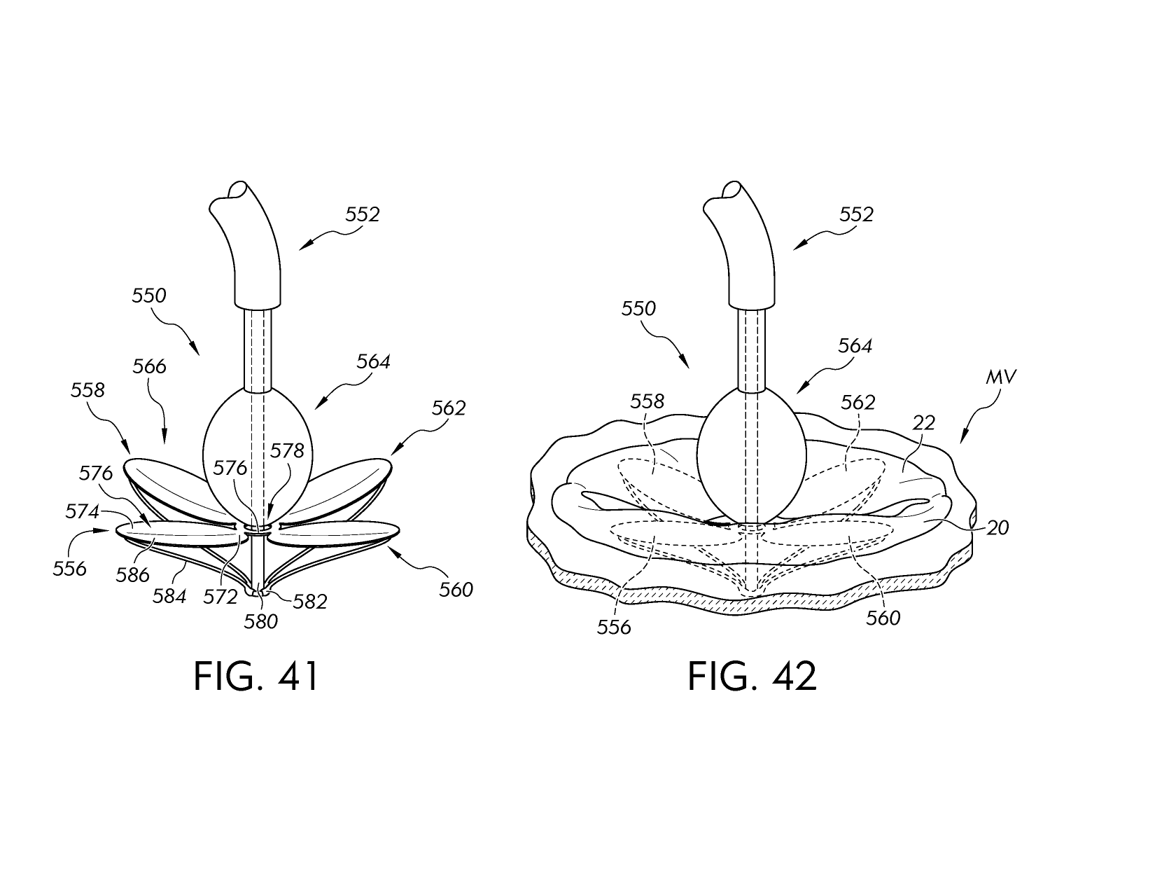

Referring to FIGS. 41-44, an example leaflet attachment device 550 is illustrated. The leaflet attachment device 550 is configured to be delivered to the location of a native heart valve (e.g., mitral valve, tricuspid valve). The leaflet attachment device 550 can be configured to be delivered via a delivery system 552 or other means for delivery. The delivery system 552 can be any suitable delivery system. For example, the delivery system 552 can be the same as, or similar to, the delivery system 102 described above and can comprise one or more of a catheter, a sheath, a guide catheter/sheath, a delivery catheter/sheath, a steerable catheter, an implant catheter, a tube, a channel, a pathway, combinations of these, etc. In some implementations, the device 550 is disposed at the end of a catheter of the delivery system 552.

Once delivered into position, the leaflet attachment device 550 is configured to engage leaflets (e.g., leaflets 20, 22, 30, 32, 34) of the native heart valve to attach the leaflets together (i.e., directly or indirectly). In some implementations, the leaflet attachment device 550 is configured to capture a first leaflet (e.g., the anterior leaflet 20 of the mitral valve MV) and a second leaflet (e.g., the posterior leaflet 22 of the mitral valve MV) of a native heart valve. Once captured, the leaflet attachment device 550 is configured to attach (e.g., by tissue fusion, an adhesive, stitches, anchors, etc.) the first leaflet and the second leaflet together, either directly to one another or to an intermediate structure.

In some implementations, the leaflet attachment device 550 can be configured to fuse the leaflets together. In some implementations, the leaflet attachment device 550 includes a first arm 556, a second arm 558, a third arm 560, and/or a fourth arm 562. In some implementations, the leaflet attachment device 550 includes a first surface (e.g., on first arm 556 or on another component), a second surface (e.g., on second arm 558 or on another component), a third surface (e.g., on third arm 560 or on another component), and/or a fourth surface (e.g., on first arm 562 or on another component).

In some implementations, the device 550 optionally includes a coaptation element 564 (e.g., spacer, coaption element, gap filler, membrane, sheet, plug, wedge, balloon, rod, tube, etc.).

Each of the surfaces and/or arms 556, 558, 560, 562 can be configured in a variety of ways. In some implementations, the surfaces and/or arms 556, 558, 560, 562 are configured the same or similar to each other. In the illustrated example, each surface and/or arm 556, 558, 560, 562 resembles a paddle (e.g., a generally elongated, flattened or slightly curved body 566). In the illustrated example, the body 566 has an oval shape. The body 566, however, can have any suitable shape.

In some implementations, each arm 556, 558, 560, 562 includes a distal end portion 572, a proximal end portion 574, and an inner face 576 (FIG. 31) extending between the distal end portion 572 and the proximal end portion 574.

In some implementations, the distal end portions 572 of each of the arms 556, 558, 560, 562 are attached to a base 576 (e.g., a ring) at a distal end 578 of the coaptation element 564.

In some implementations, the distal end portions 572 can be attached to the distal end 578 of the coaptation element 564. The distal end portions 572 can be attached in any suitable manner that allows the arms 556, 558, 560, 562 to pivot relative to the coaptation element 564. For example, in some implementations, the distal end portion 572 includes a flexible or bendable portion that allow each arm 556, 558, 560, 562 to bend or flex at the flexible or bendable portion to pivot relative to the coaptation element 564.

The arms 556, 558, 560, 562 can be moved between the open condition and the closed condition by any suitable means. In some implementations, an actuation element 580 (e.g., actuation wire, shaft, tube, hypotube, line, suture, braid, etc.) can be provided to move the arms 556, 558, 560, 562 between the open condition and the closed condition. In the illustrated example, the actuation element 580 extends through the coaptation element 564 and is attached to an end cap 582 distal to the coaptation element 564.

In some implementations, the device 550 includes a plurality of links 584 (e.g., strip, rod, arm, band, bar, ribbon, line, etc.), each link 584 associated with a corresponding one of the arms 556, 558, 560, 562. Each link 584 is attached to the end cap 582 on one link end and attached to the proximal end portion 574 of a corresponding arm 556, 558, 560, 562 at the opposite link end.

In some implementations, to move between the open condition and closed condition, the actuation element 580 can be moved axially relative to the coaptation element 564. For example, moving the actuation element 580 distally moves the end cap 582 distally and pivots the arms 556, 558, 560, 562 toward the open condition via the links 584. Likewise, moving the actuation element 580 proximally can move the end cap 582 proximally and pivots the arms 556, 558, 560, 562 toward the closed condition via the links 584.

In some implementations, the inner face 576 of each of the arms 556, 558, 560, 562 includes a contact surface 586 (e.g., pad, ridge, projection, block, or other suitable surface) configured to contact a leaflet when the leaflet is grasped.

In some implementations, the contact surface 586 is configured to output energy (e.g., heat up in response to radiofrequency, ultrasonic, or other suitable energy) to fuse the leaflets together.

In some implementations, the contact surface 586 can be a continuous surface that extends along a portion of the inner surface 576. In some implementations, the contact surface 586 can be discontinuous (e.g., a plurality of discrete surfaces on the inner face 576) or can extend the entire length of the inner face 576.

In some implementations, the coaptation element 564 is configured to extend between the arms 556, 558, 560, 562 in the closed condition. In some implementations, the center element 510 allows the arms 556, 558, 560, 562 to capture the leaflets 20, 22 against the center element 510. In some implementations, the center element 510 can be a dynamic member that can be retracted (i.e., moved out from between the captured leaflets) or have a geometry that allows more direct compression between the contact surfaces 586 when the leaflets are captured.

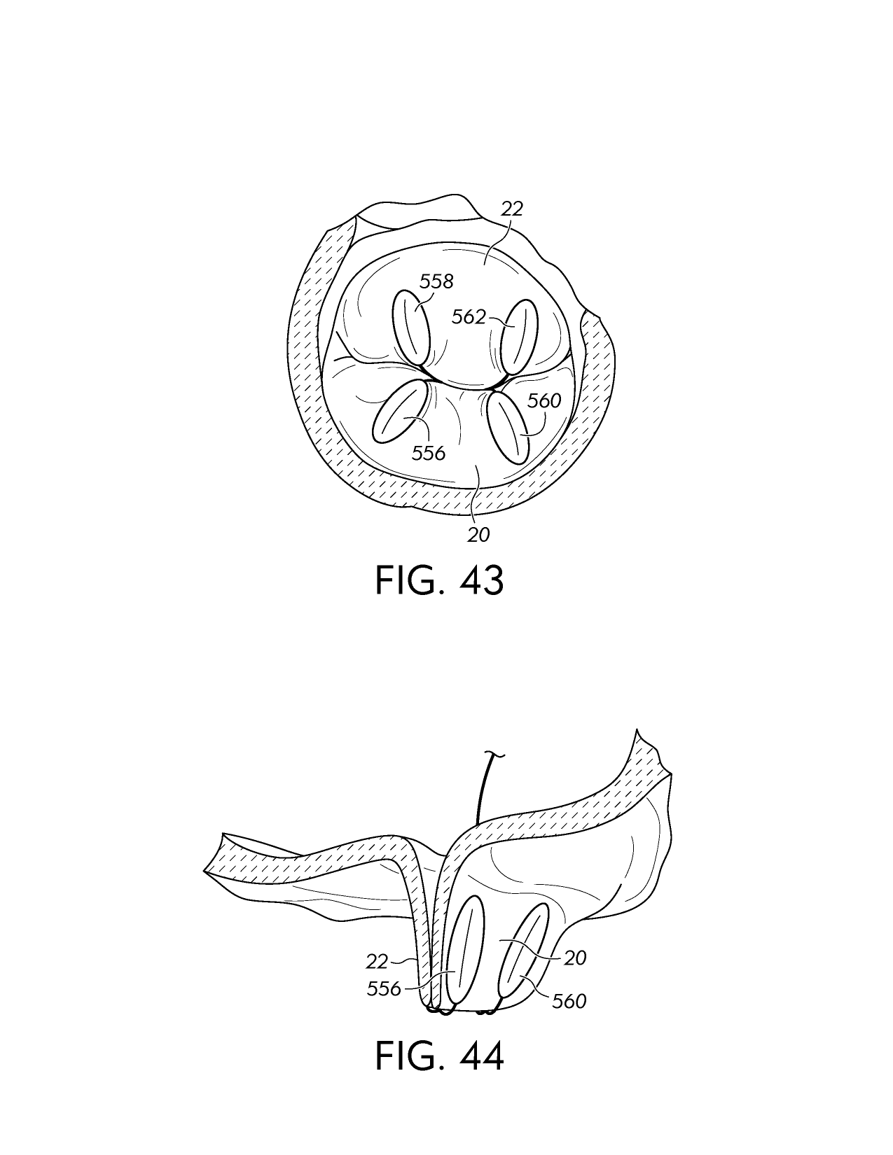

FIGS. 42-43 illustrates the example leaflet attachment device 550 in the open condition in position within the mitral valve MV. The leaflets 20, 22 are positioned between the coaptation element 564 and the arms 556, 558, 560, 562. Once the leaflet attachment device 550 is properly positioned, the arms 556, 558, 560, 562 can be moved to the closed condition to capture leaflets, as shown in FIG. 44. With the leaflets 20, 22 captured, the leaflet attachment device 550 can fuse the leaflets 20, 22 together by energizing the contact surfaces 586.

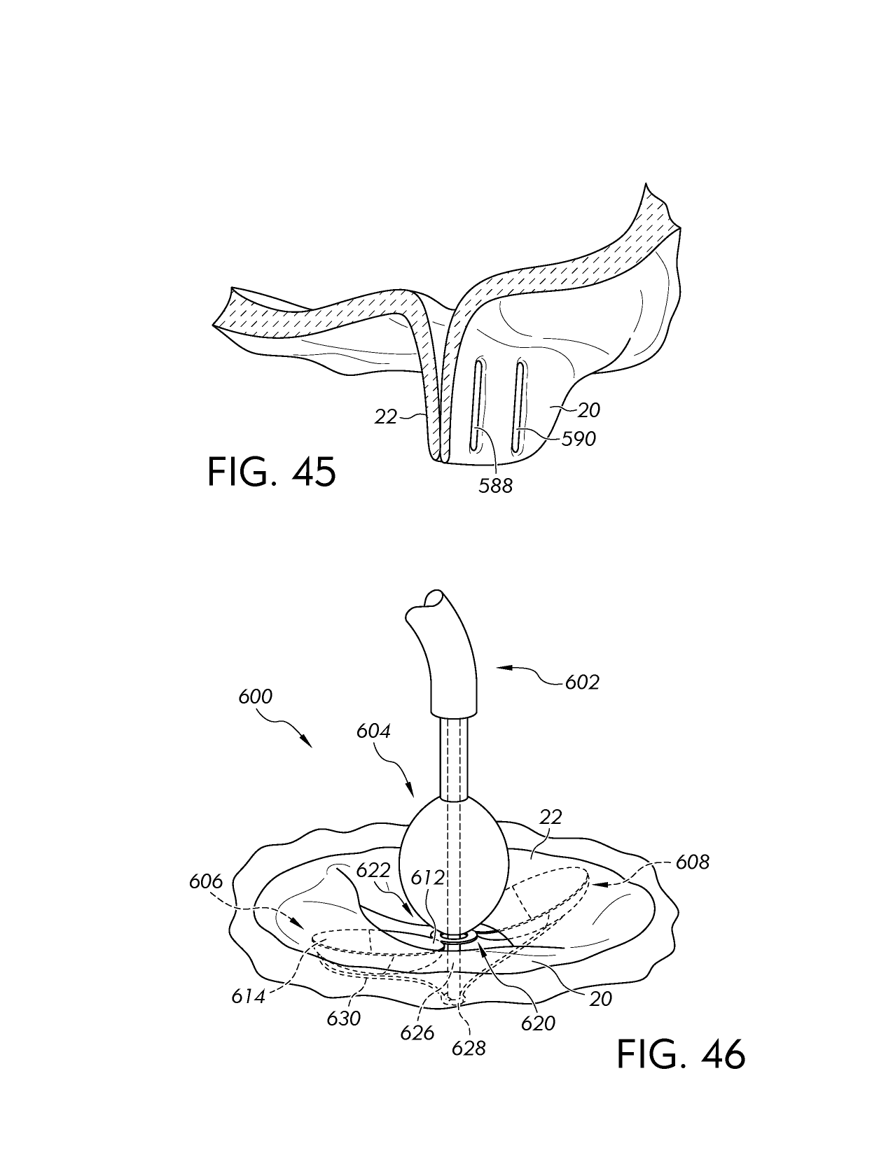

In the illustrated example, the first arm 556 and the second arm 558 fuse the leaflets 20, 22 together at a first location (i.e., a first fused section 588) and the third arm 560 and the fourth arm 562 fuse the leaflets 20, 22 together in a second location (i.e., a second fused section 590) spaced apart from the first location, as shown in FIG. 45. Thus, there is unfused section between the leaflets that is between a first fused section 588 and the second fused section 590. In some implementations, the unfused section can provide an opening in which the leaflet attachment device 550 can be withdrawn.

Referring to FIGS. 34A and 45A, in some implementations, any of the leaflet attachment devices can be configured to provide a single fused section 590 between the leaflets, instead of a pair of spaced apart fused sections. This allows the leaflet attachment device to be removed after the single sealed fused is made by opening the leaflet attachment device, moving the device laterally relative to the single sealed section, and or retracting the center member or coaptation element. The leaflet attachment device does not need to by pulled through an opening between a pair of spaced apart fused sections, so withdrawing the device through the treated valve is easier.

In the example illustrated by FIG. 34A, the device 500 is a modified version of the device illustrated by FIG. 34. However, any of the devices disclosed herein can be modified to provide a single fused section to accommodate easier withdrawal of the device, instead of a pair of spaced apart fussed sections. For example, any of the devices disclosed herein that have two spaced apart pairs of fusing members can be modified to have only one pair of fusing members that are optionally offset to one side (see FIG. 34A) of the device. The devices work in the same or similar way to the other devices disclosed herein, except that a single seal is formed, optionally to one side of the device, instead of a pair of seals around a center member or coaptation element.

In the example illustrated by FIG. 34A, each of the first and second arms 506, 508 are generally L-shaped. For example, the first arm 506 includes a first projecting portion 522 and a portion 526. The contact surfaces 512 are positioned at the distal end 530 of the first projecting portion 522. In the illustrated examples, the second arm 508 is configured the same as, or like, the first arm 506 and arranged in a mirror image to the first arm 506 as shown in FIG. 34A. When the first arm 506 is in the closed condition, the captured first leaflet is pinched between the first arm 506 and the center element 510. Likewise, when the second arm 508 is in the closed condition, the captured second leaflet is pinched between the second arm 508 and the center element 510. With both the first and the second arms 506, 508 in the closed condition, the first and second leaflets are also pinched between the contact surfaces 512 on the projecting portions 522 of the first and second arms 506, 508.

In some implementations, once the first and second leaflets are pinched between the contact surfaces 512 of the first and second arms 506, 508, the leaflet attachment device 500 can be energized such that the contact surfaces 512 fuse the leaflets together in the area between the contact surfaces 512 to form a fused area 590 (see FIG. 45A). In some implementations, the center element 510 is still in place between the first and the second arms 506, 508 when the leaflets are fused together. In other implementations, the center element 510 is retractable and can be removed prior to fusing the leaflets together.

In some implementations, the attachment device 500 is configured and attached to the delivery device, so that the attachment device is rotated 180 degrees from the positions illustrated by FIGS. 31-33, 38-40, and 40A. For example, the attachment device 500, optionally with a single pair of contact surfaces 512, can be oriented and/or operated in the same or similar manner to the device 100 illustrated by FIGS. 8-21, and can be configured, oriented and/or operated in the same or similar manner to the device 550 illustrated by FIGS. 41-44 (but with only a single pair of arms 556, 558, optionally positioned to one side of the device 550). These configurations allow the attachment devices to be delivered through the valves (e.g., mitral valve or tricuspid valve), grasp the leaflets, fuse the leaflets together, move the device laterally in the valve, and then retract the device back through the valve.

In some implementations, multiple fused sections 590 can be formed sequentially, optionally with a device having a single pair of contact surfaces. For example, the attachment devices can be delivered through the valves (e.g., mitral valve or tricuspid valve), grasp the leaflets, fuse the leaflets together at a first location, move the device laterally in the valve, fuse the leaflets together at a second location, move the device laterally in the valve and then retract the device back through the valve. Any number of fused portions can be formed. Any of the devices disclosed herein can be configured to form multiple fused sections in this way.

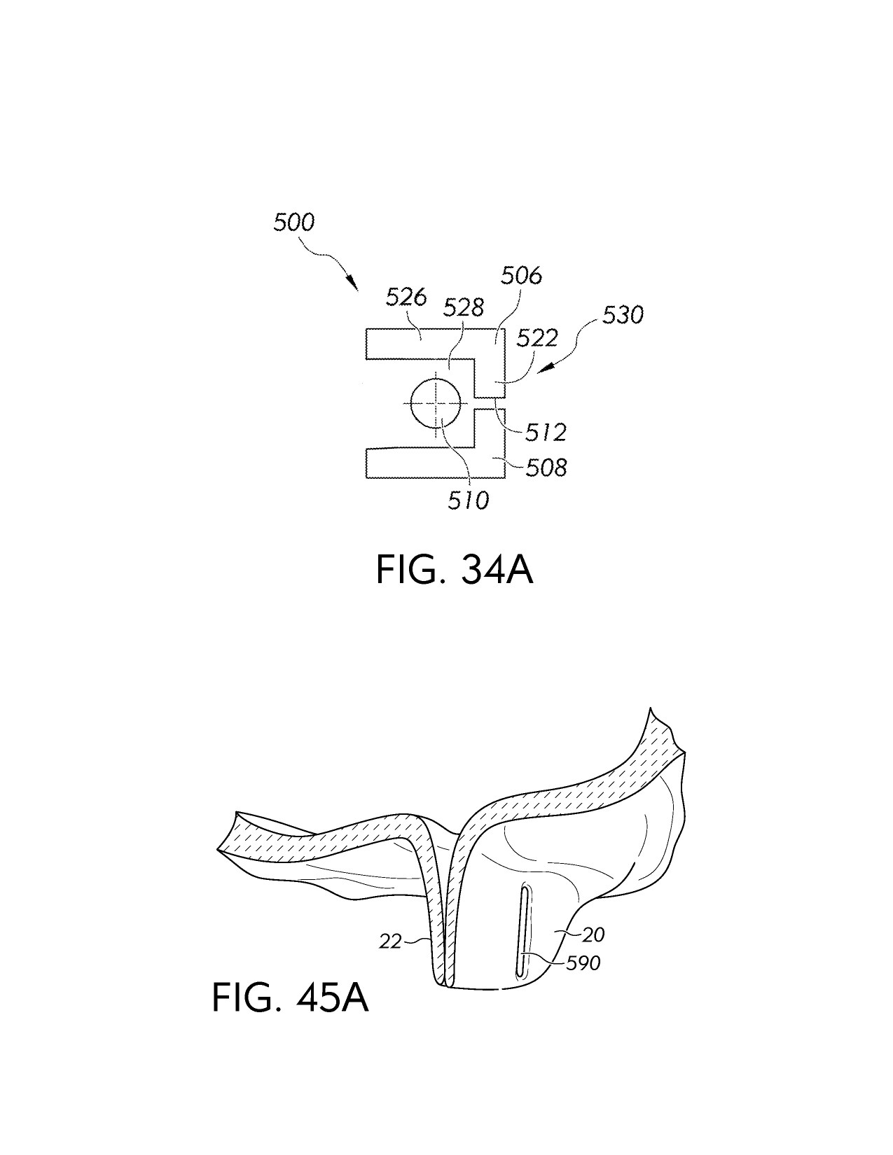

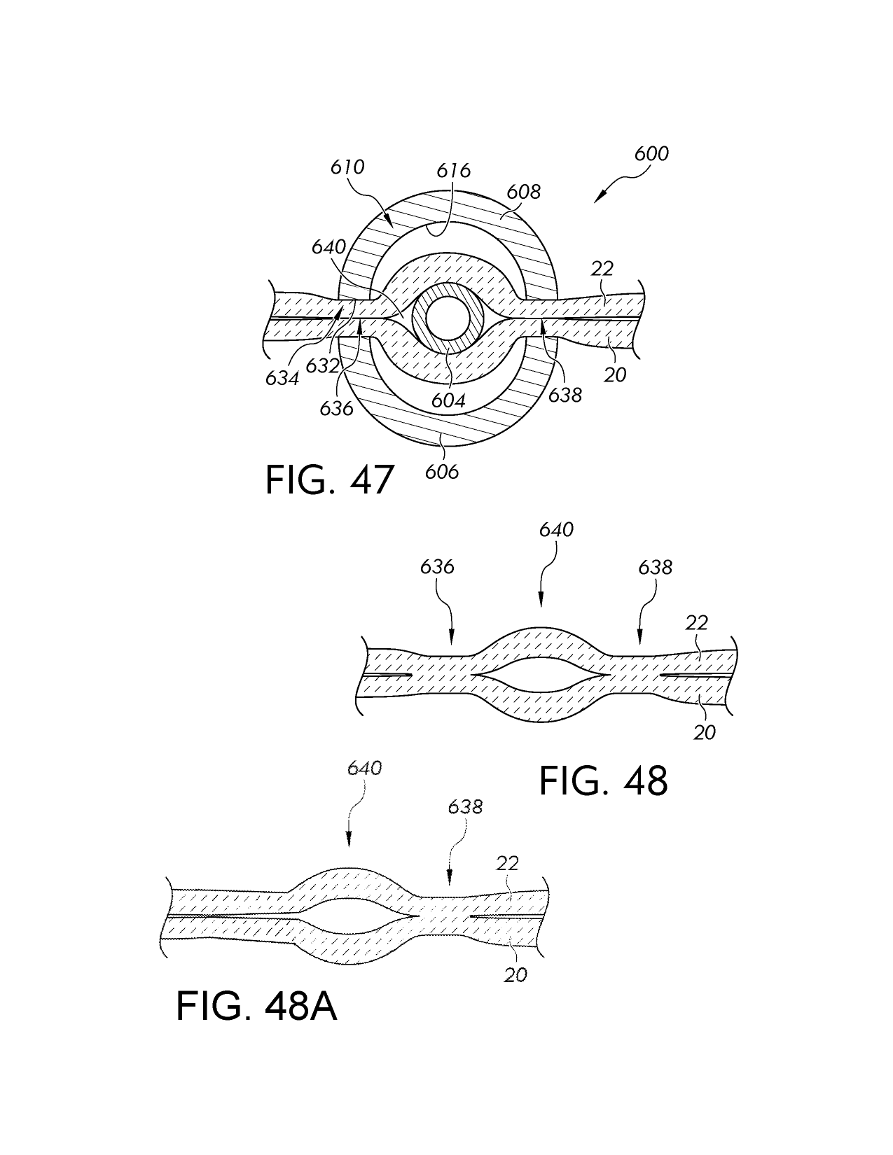

FIGS. 46-47, an example leaflet attachment device 600 is illustrated. The leaflet attachment device 600 is configured to be delivered to the location of a native heart valve (e.g., mitral valve, tricuspid valve). The leaflet attachment device 600 can be configured to be delivered via a delivery system 602 or other means for delivery. The delivery system 602 can be any suitable delivery system. For example, the delivery system 602 can be the same as, or similar to, the delivery system 102 described above and can comprise one or more of a catheter, a sheath, a guide catheter/sheath, a delivery catheter/sheath, a steerable catheter, an implant catheter, a tube, a channel, a pathway, combinations of these, etc. In some implementations, the device 600 is disposed at the end of a catheter of the delivery system 602.

Once delivered into position, the leaflet attachment device 600 is configured to engage leaflets (e.g., leaflets 20, 22, 30, 32, 34) of the native heart valve to attach the leaflets together (i.e., directly or indirectly). In some implementations, the leaflet attachment device 600 is configured to capture a first leaflet (e.g., the anterior leaflet 20 of the mitral valve MV) and a second leaflet (e.g., the posterior leaflet 22 of the mitral valve MV) of a native heart valve.

In some implementations, once captured, the leaflet attachment device 600 is configured to attach (e.g., by tissue fusion, an adhesive, stitches, anchors, etc.) the first leaflet and the second leaflet together, either directly to one another or to an intermediate structure. In some implementations, the leaflet attachment device 600 can be configured to fuse the leaflets together.

In some implementations, the leaflet attachment device 600 includes a coaptation element 604 (e.g., spacer, coaption element, gap filler, membrane, sheet, plug, wedge, balloon, rod, tube, etc.), first arm 606, a second arm 608.

The first arm 606 and the second arm 608 can be configured in a variety of ways. In some implementations, the first arm 606 and the second arm are configured the same or similar to each other. In the illustrated example, each of the first arm 606 and the second arm resemble a paddle having an elongated body 610 having a C-shaped cross-section. In the illustrated example, the body 610 has an oval shape. The body 610, however, can have any suitable shape.

In some implementations, each of the first arm 606 and the second arm 608 includes a proximal end portion 612, a distal end portion 614, and an inner face 616 (FIG. 46) extending between the proximal end portion 612 and the distal end portion 614. In some implementations, the inner face 616 is concave.

In some implementations, the proximal end portions 612 of each of the arms 606, 608 are attached to a base 620 (e.g., a ring) at a distal end 622 of the coaptation element 604. In some implementations, the proximal end portions 612 can be attached to the distal end 622 of the coaptation element 604. The proximal end portions 612 can be attached in any suitable manner that allows the arms 606, 608 to pivot relative to the coaptation element 604. For example, in some implementations, the proximal end portion 612 includes a flexible or bendable portion that allow each arm 606, 608 to bend or flex at the flexible or bendable portion to pivot relative to the coaptation element 604.

The first arm 606 and the second arm 608 can be moved between the open condition and the closed condition by any suitable means. In some implementations, an actuation element 626 (e.g., actuation wire, shaft, tube, hypotube, line, suture, braid, etc.) can be provided to move the first arm 606 and the second arm 608 between the open condition and the closed condition. In the illustrated example, the actuation element 626 extends through the coaptation element 604 and is attached to an end cap 628 distal to the coaptation element 604.

In some implementations, the device 600 includes a plurality of links 630 (e.g., strip, rod, arm, band, bar, ribbon, etc.), each link 630 associated with a corresponding one of the arms 606, 608. Each link 630 is attached to the end cap 628 on one link end and attached to the distal end portion 614 of a corresponding arm 606, 608 at the opposite link end.

In some implementations, to move between the open condition and closed condition, the actuation element 626 can be moved axially relative to the coaptation element 604. For example, moving the actuation element 626 distally moves the end cap 628 distally and pivots the arms 606, 608 toward the open condition via the links 630. Likewise, moving the actuation element 626 proximally moves the end cap 628 proximally and pivots the arms 606, 608 toward the closed condition via the links 630.

In some implementations, each the arms 606, 608 include one or more contact surfaces 632 (e.g., pad, ridge, projection, block, plane, or other suitable surface) configured to contact a leaflet when the leaflet is grasped. The contact surfaces 632 can be configured to output energy (e.g., heat up in response to radiofrequency, ultrasonic, or other suitable energy) to fuse the leaflets together. In some implementations, contact surfaces 632 are located at, or define, end surfaces 634 on the arms 606, 608.

In some implementations, the contact surface 632 can be a continuous surface that extends along a portion of the end surfaces 634. In some implementations, the contact surface 632 can be discontinuous (e.g., a plurality of discrete surfaces on the end surfaces 634) or can extend the entire length of the end surfaces 634.

In some implementations, the coaptation element 604 is configured to extend between the arms 606, 608 in the closed condition. In some implementations, the coaptation element 604 allows the arms 606, 068 to capture the leaflets 20, 22 against the coaptation element 604. In some implementations, the coaptation element 604 can be a dynamic member that can be retracted (i.e., moved out from between the captured leaflets) or have a geometry that allows more direct compression between the contact surfaces 632 when the leaflets are captured.

Referring to FIGS. 47-48, in the illustrated example, the first arm 606 and the second arm 608 fuse the leaflets 20, 22 together at a first location (i.e., a first fused section 636) and at a second location (i.e., a second fused section 638) spaced apart from the first location. Thus, there is an unfused section 640 between the leaflets that is between a first fused section 636 and the second fused section 638. In some implementations, the unfused section can provide an opening in which the leaflet attachment device 600 can be withdrawn after the leaflets have been fused.

Referring to FIG. 48A, in some implementations, the first arm 606 and the second arm 608 fuse the leaflets 20, 22 together at only a single location (e.g., fused section 638) spaced apart from the first location. As described above, fusing the leaflets together at a single location can allow the device to be more easily removed and/or can allow for a plurality of fused sections to be formed sequentially.

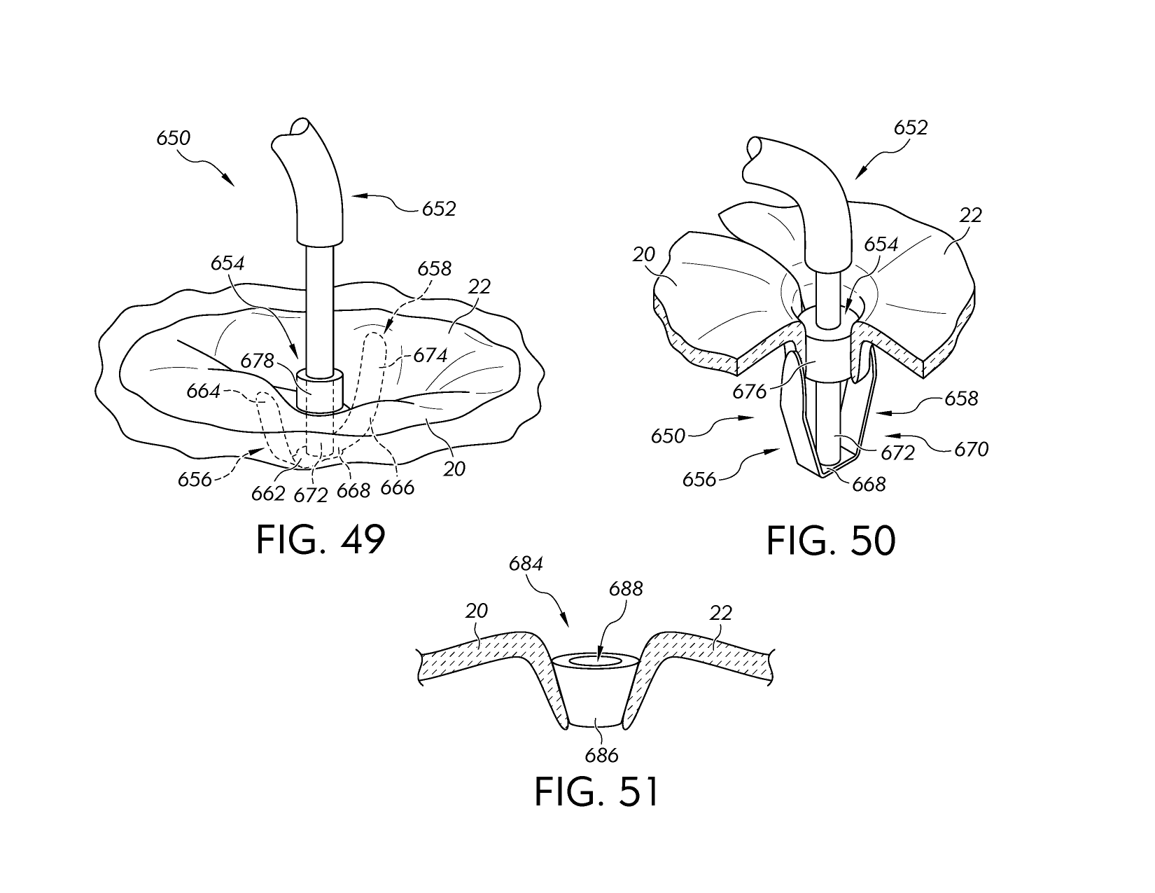

Referring to FIGS. 49-50, an example leaflet attachment device 650 is illustrated. The leaflet attachment device 650 is configured to be delivered to the location of a native heart valve (e.g., mitral valve, tricuspid valve). The leaflet attachment device 650 can be configured to be delivered via a delivery system 652 or other means for delivery. The delivery system 652 can be any suitable delivery system. For example, the delivery system 652 can be the same as, or similar to any delivery system described herein (e.g., delivery system 102) and can comprise one or more of a catheter, a sheath, a guide catheter/sheath, a delivery catheter/sheath, a steerable catheter, an implant catheter, a tube, a channel, a pathway, combinations of these, etc. In some implementations, the device 650 is disposed at the end of a catheter of the delivery system 652.

In some implementations, once delivered into position, the leaflet attachment device 650 is configured to engage leaflets (e.g., leaflets 20, 22, 30, 32, 34) of the native heart valve to attach the leaflets together (i.e., directly or indirectly). In some implementations, the leaflet attachment device 650 is configured to capture a first leaflet (e.g., the anterior leaflet 20 of the mitral valve MV) and a second leaflet (e.g., the posterior leaflet 22 of the mitral valve MV) of a native heart valve.

In some implementations, once captured, the leaflet attachment device 650 is configured to attach (e.g., by tissue fusion, an adhesive, stitches, anchors, etc.) the first leaflet and the second leaflet together, either directly to one another or to an intermediate structure. In the illustrated example, the leaflet attachment device 650 includes a coaptation element 654 (e.g., spacer, coaption element, gap filler, membrane, sheet, plug, wedge, balloon, rod, tube, etc.), first arm 656, and a second arm 658.

The first arm 656 and the second arm 658 can be configured in a variety of ways. In some implementations, the first arm 656 and the second arm 658 are configured the same or similar to each other. In the illustrated example, each of the first arm 656 and the second arm 658 resemble a paddle having an elongated, flattened body. The first arm 656 and the second arm 658, however, can have any suitable shape.

In some implementations, each of the first arm 656 and the second arm 658 includes a proximal end portion 662, a distal end portion 664, and an inner face 666 extending between the proximal end portion 662 and the distal end portion 664. In some implementations, the proximal end portions 662 of each of the arms 656, 658 are attached to a base 668 at a distal end 670 of the device 650.

The proximal end portions 662 can be attached in any suitable manner that allows the arms 656, 658 to pivot relative to the coaptation element 654. For example, in some implementations, the proximal end portion 662 includes a flexible or bendable portion that allow each arm 656, 658 to bend or flex at the flexible or bendable portion to pivot relative to the coaptation element 654.

The first arm 656 and the second arm 658 can be moved between the open condition and the closed condition by any suitable means. In some implementations, an actuation element 672 (e.g., actuation wire, shaft, tube, hypotube, line, suture, braid, etc.) can be provided to move the first arm 656 and the second arm 658 between the open condition and the closed condition. Any suitable actuation element 672 can be used, such as for example, any actuation element disclosed herein.

In some implementations, each of the arms 656, 658 include one or more contact surfaces 674 (e.g., pad, ridge, projection, block, plane, or other suitable surface) configured to contact a leaflet when the leaflet is grasped. In some implementations, the contact surfaces 674 can be configured to output energy (e.g., heat up in response to radiofrequency, ultrasonic or other suitable energy) to fuse the leaflets. In some implementations, contact surfaces 674 are located on the inner face 666 of the first arm 656 and second arm 658.

In some implementations, the contact surface 674 can be a continuous surface that extends along a portion of the inner face 666. In some implementations, the contact surface 674 can be discontinuous (e.g., a plurality of discrete surfaces on the end surfaces 634) or can extend the entire length of the inner face 666.

In some implementations, the coaptation element 654 is configured to extend between the arms 656, 658 in the closed condition. In some implementations, the coaptation element 604 allows the arms 656, 658 to capture the leaflets 20, 22 against the coaptation element 654.

In some implementations, the device 650 is configured to fuse the leaflets 20, 22 to the coaptation element 654, in addition to or as an alternative to, fusing the leaflets 20, 22 directly together. For example, the coaptation element 654 can include collagen and/or elastin tissue that can be fused to the leaflets 20, 22. Thus, as shown in FIG. 50, the leaflets 20, 22 can be fused directly onto an exterior surface 676 of the coaptation element 654.

FIG. 49 illustrates the device 650 in position in the mitral valve with the first arm 656 and the second arm 658 in position to capture the leaflets 20, 22 against the coaptation element 654. FIG. 50 illustrates the first arm 656 and the second arm 658 having captured the leaflets 20, 22 against the coaptation element 654 and fusing the leaflets 20, 22 to the exterior surface 676 of the coaptation element 654. In the illustrated example, the exterior surface 676 of the coaptation element 654 is cylindrical. In some implementations, however, the exterior surface 676 can be shaped other than cylindrical. In some implementations, the coaptation element 654 is annular.

In the illustrated example, the coaptation element 654 includes a longitudinal passage 678. In some implementations, the actuation element 672 extends through the longitudinal passage 678. In some implementations, the longitudinal passage 678 is configured to allow the device (e.g., the first arm 656 and the second arm 658) to be withdrawn through the longitudinal passage 678 after the leaflets 20, 22 have been fused to the coaptation element 654.

Referring to FIG. 51, an example coaptation element 684 useable and/or for use with a leaflet attachment device (e.g., leaflet attachment device 650) is illustrated. The coaptation element 684 can be substantially like the coaptation element 654 of FIGS. 49-50. For example, the coaptation element 684 is configured such that native valve leaflets (e.g., leaflets 20, 22) can be fused to the coaptation element 654. Thus, in some implementations, the coaptation element 658 includes collagen and/or elastin tissue that can be fused to the leaflets 20, 22.

In the illustrated example, the coaptation element 684 is annular and includes an exterior surface 686 and a longitudinal passage 688. In some implementations, the exterior surface 686 is configured to fuse to the leaflets 20, 22 and the longitudinal passage 688 can be configured to receive an actuation member therethrough and allow for the leaflet attachment device to be withdrawn through longitudinal passage 688 after the leaflets 20, 22 have been fused to the exterior surface 686. While the exterior surface 676 of the illustrated coaptation element 654 is cylindrical, the exterior surface 686 of the coaptation element 684 can be conical (i.e., tapers inward in the distal direction).

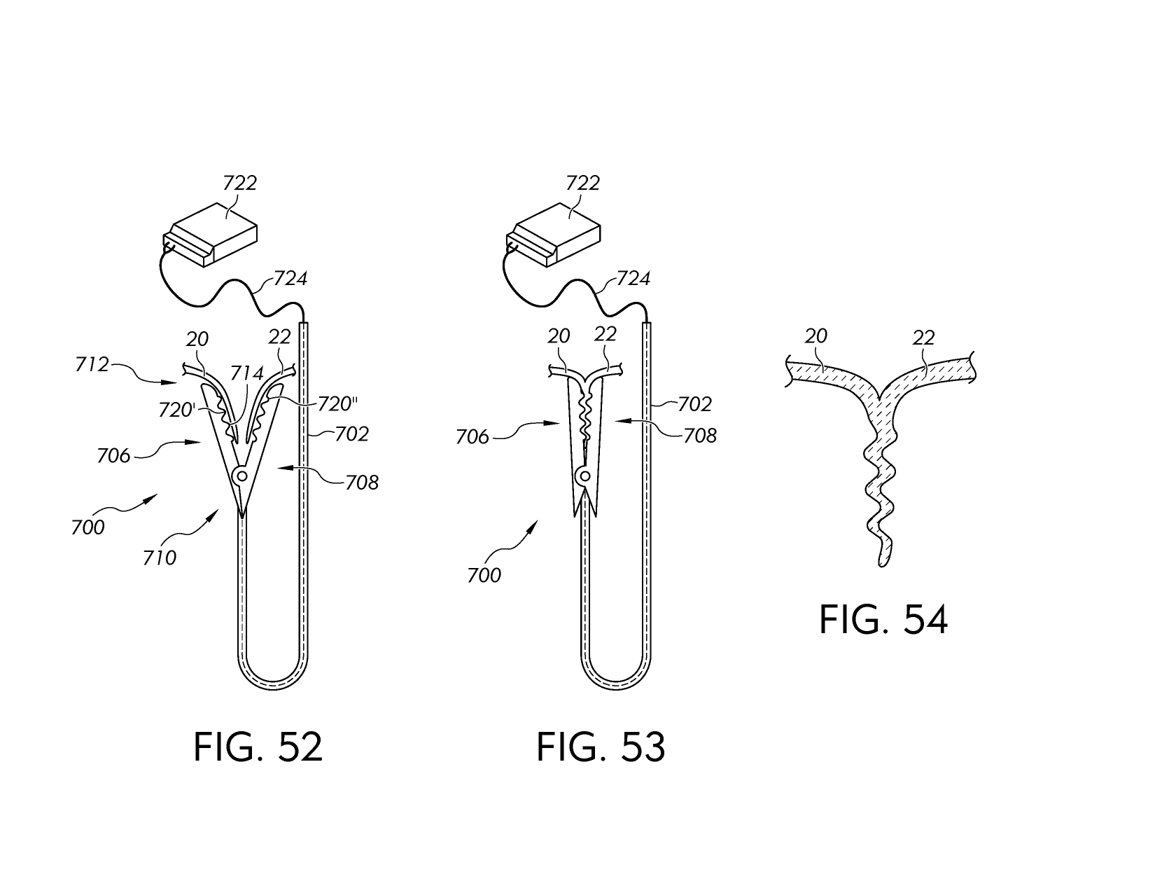

FIGS. 52-53 schematically illustrate an example leaflet attachment device 700. The leaflet attachment device 700 is configured to be delivered to the location of a native heart valve (e.g., mitral valve, tricuspid valve). The leaflet attachment device 700 can be configured to be delivered via a delivery system 702 or other means for delivery. The delivery system 702 can be any suitable delivery system. For example, the delivery system 702 can be the same as, or similar to any delivery system described herein (e.g., delivery system 102) and can comprise one or more of a catheter, a sheath, a guide catheter/sheath, a delivery catheter/sheath, a steerable catheter, an implant catheter, a tube, a channel, a pathway, combinations of these, etc. In some implementations, the device 700 is disposed at the end of a catheter of the delivery system 702.

In some implementations, once delivered into position, the leaflet attachment device 700 is configured to engage leaflets (e.g., leaflets 20, 22, 30, 32, 34) of the native heart valve to attach the leaflets together (i.e., directly or indirectly). In some implementations, the leaflet attachment device 700 is configured to capture a first leaflet (e.g., the anterior leaflet 20 of the mitral valve MV) and a second leaflet (e.g., the posterior leaflet 22 of the mitral valve MV) of a native heart valve.

In some implementations, once captured, the leaflet attachment device 700 is configured to attach (e.g., by tissue fusion, an adhesive, stitches, anchors, etc.) the first leaflet and the second leaflet together, either directly to one another or to an intermediate structure. In the illustrated example, the leaflet attachment device 700 includes a first arm 706 and a second arm 708. In some implementations, the leaflet attachment device 700 can include a coaptation element, such as any coaptation element disclosed herein.

The first arm 706 and the second arm 708 can be configured in a variety of ways. In some implementations, the first arm 706 and the second arm 708 are configured the same or similar to each other. In some implementations, each of the first arm 706 and the second arm 708 includes a proximal end portion 710, a distal end portion 712, and an inner face 714 extending between the proximal end portion 710 and the distal end portion 712.

In some implementations, the first arm 706 and the second arm 708 are configured to move between an open condition (FIG. 52) and a closed condition (FIG. 53). The first arm 706 and the second arm 708 can be moved between the open condition and the closed condition by any suitable means, such as any actuation member or means disclosed herein. In some implementations, the first arm 706 and the second arm 708 are pivotal relative to each other.

In some implementations, the first arm 706 and the second arm 708 include one or more contact surfaces 720 (e.g., pad, ridge, projection, block, plane, or other suitable surface) configured to contact a leaflet when the leaflet is grasped. The contact surfaces 720 can be configured to output energy (e.g., heat up in response to radiofrequency, ultrasonic or other suitable energy) to fuse the leaflets. In some implementations, contact surfaces 720 are located on the inner face 714 of the first arm 706 and the second arm 708. The contact surface 720 can be a continuous surface that extends along a portion of the inner face 714. In other implementations, the contact surface 720 can be discontinuous or can extend the entire length of the inner face 714.

In some implementations, the inner face 714 and/or the one or more contact surfaces 720 includes have an uneven or textured surface configuration (e.g., serrations, bumps, dimples, projections, ridges, protuberances, undulations, etc.). In some implementations, the uneven or textured surface configuration of the first arm 706 can be complementary to the uneven or textured surface configuration of the second arm 708. In the illustrated example, the first arm 706 include a wavy or serrated contact surface 720’ and the second arm include a complementary wavy or serrated contact surface 720’’ that is offset such that a convex portion (i.e., a peak) of the wavy or serrated contact surface 720’ of the first arm 706 is aligned with a concave portion (i.e., a valley) of the of the wavy or serrated contact surface 720’’ of the second arm 708. As a result, when the first arm 706 and the second arm 708 fuse the leaflets 20, 22 together, the resulting fused area between the leaflets is contoured (FIG. 54) due to the wavy or serrated profile of the contact surfaces 720’, 720’ which results in a stronger bond.

In the illustrated example, the leaflet attachment device 700 is coupled to an energy source 722 capable of supplying energy to the leaflet attachment device 700 that is sufficient to fuse the leaflets. Any suitable energy source 722 can be used, such as, for example a radio frequency generator, an ultrasonic energy generator, etc. In some implementations, the energy source 722 can be coupled to the leaflet attachment device 700 by a line 724 (e.g., cable, wire, cord, etc.) configured to transmit energy from the energy source 722 to the leaflet attachment device 700 (e.g., to the contact surfaces of the leaflet attachment device). In some implementations, the line 724 can extend from the energy source 722 to the leaflet attachment device 700 through the delivery system 702.

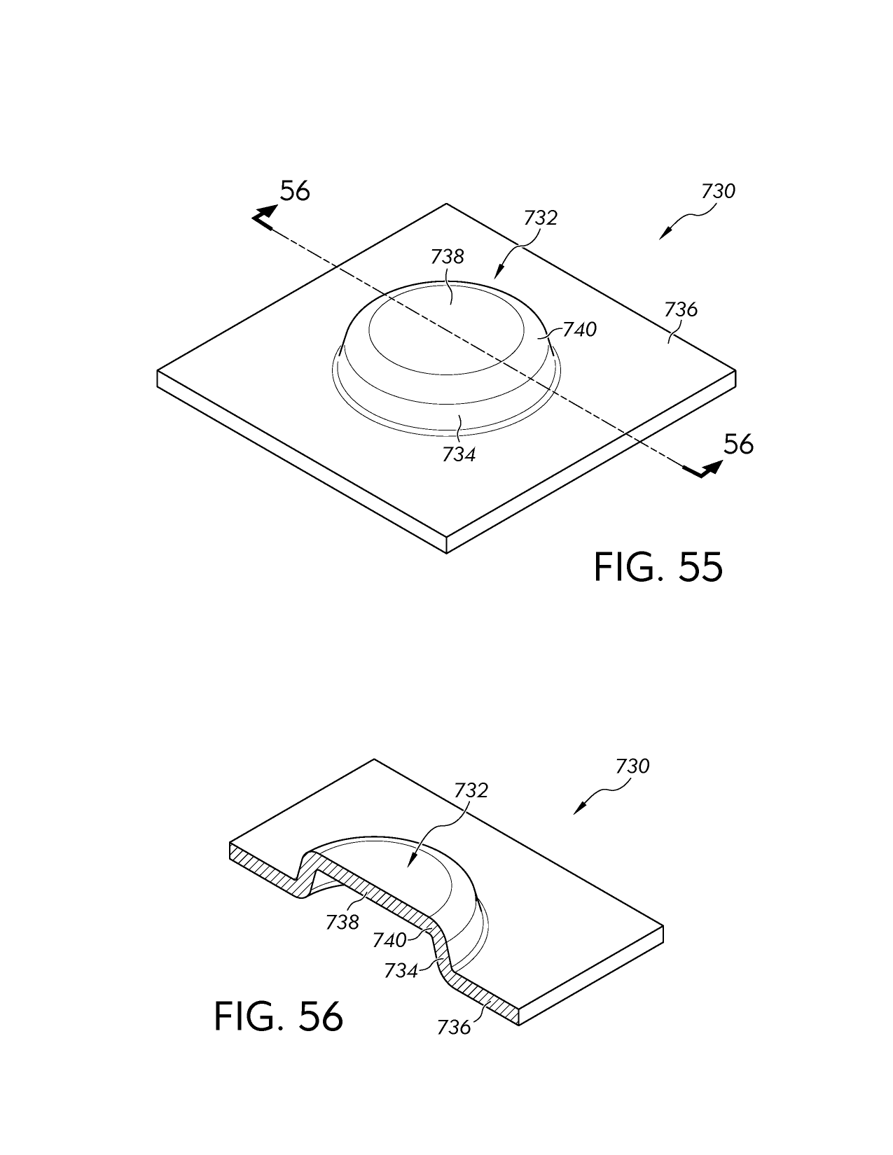

FIGS. 55-56 illustrate an example uneven or textured surface configuration of the inner face and/or the one or more contact surfaces of any of the leaflet attachment devices disclosed herein. In some implementations, the surface configuration of the inner face and/or the one or more contact surfaces can be formed from one or more surface attachments 730. In the illustrated example, a single surface attachment 730 is shown having a single projection 732 (e.g., dimple, bumps, protuberances, etc.). In some implementations, the surface attachment 730 can have a plurality of spaced-apart projections.

In some implementations, the entire inner face and/or contact surface is formed by a single surface attachment 730 having a plurality of spaced-apart projections. In some implementations, the plurality of spaced-apart projections can be integrally formed with an arm of the leaflet attachment device.

The projection(s) 732 can be configured in a variety of ways. In the illustrated example, the projection 732 is a round dome having a side wall 734 extending outward from a base 736, a top wall 738 generally parallel with the base 736, and a curved wall 740 connecting the side wall 734 to the top wall 738.

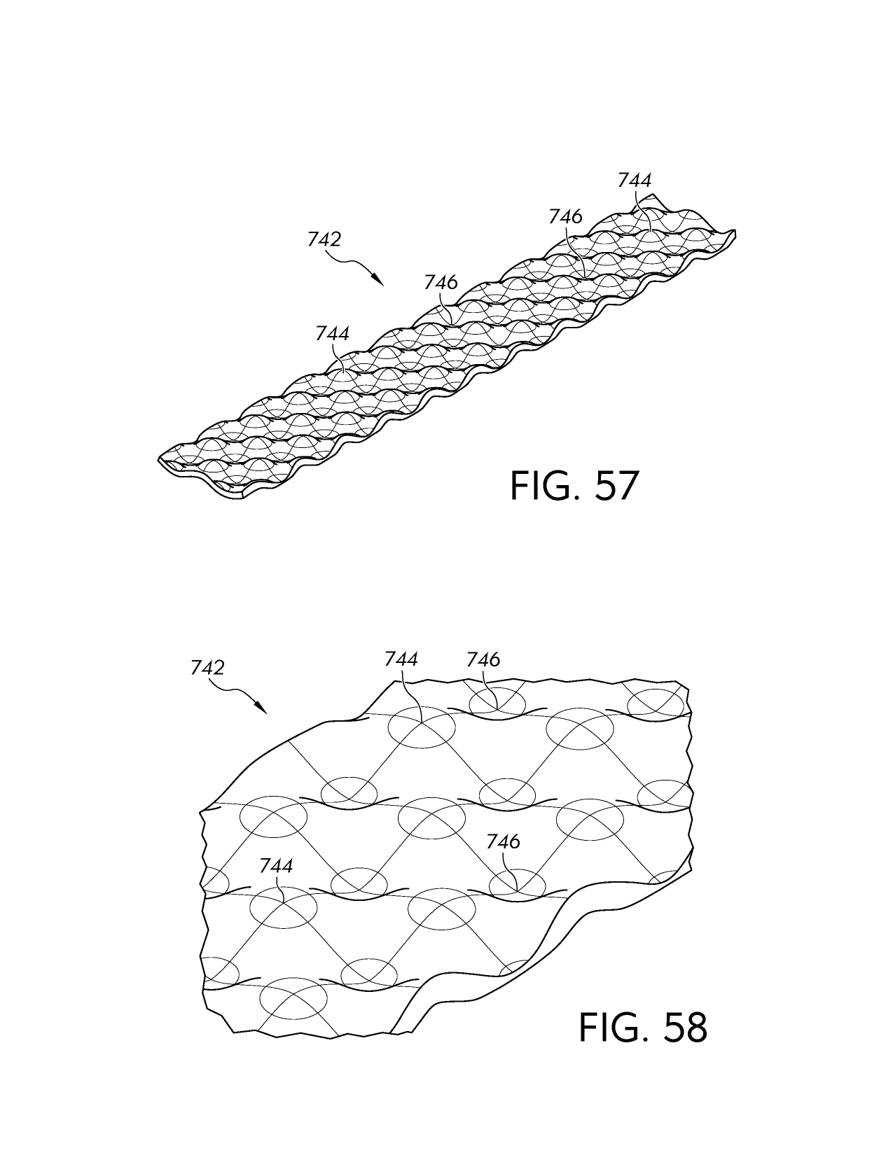

Referring to FIGS. 57-58, the leaflets can be clamped between the contact surfaces of two arms of a leaflet attachment device and fused together. In some implementations, the inner face and/or the one or more contact surfaces of the leaflet attachment device can include a plurality of the projections 732 to create a contoured fused area or surface 742 between leaflets. As with the wavy or serrated contact surface 720’, 720’’, the contact surfaces having the plurality of projections 730 can be offset and complementary to create a contoured, undulating fused area or surface 742 with convex portions 744 (i.e., peaks) and concave portions 746 (i.e., valleys) formed by the projections 730, which results in a stronger bond.

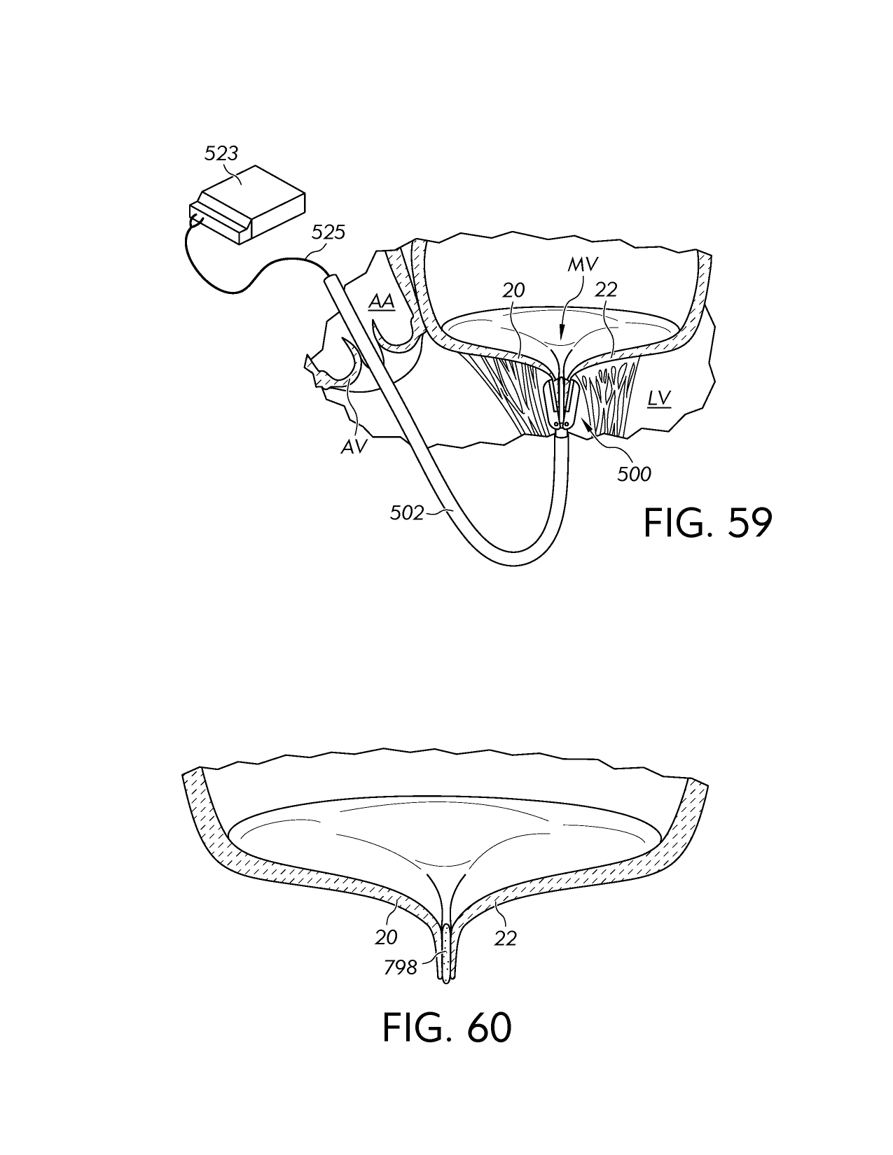

FIG. 59 illustrates the tissue attachment device 500 of FIGS. 31-40 deployed at the mitral valve MV. In particular, the tissue attachment device 500 is disposed at the end of a catheter of the delivery system 502. In the illustrated example, the tissue attachment device 500 is delivered to the mitral valve MV from the ascending aorta AA. In particular, the catheter of the delivery system 502 can be extended through the aortic valve AV and into the left ventricle LV. In some implementations, while in the left ventricle LV, the catheter can be steered to position the tissue attachment device 500 below the mitral valve MV and capture the leaflets of the mitral valve MV, as shown in FIG. 59.

In the illustrated example, the leaflet attachment device 500 is coupled to an energy source 523 capable of supplying energy to the leaflet attachment device 500 that is sufficient to fuse the leaflets together 20, 22. Any suitable energy source 523 can be used, such as, for example, a radio frequency generator, an ultrasonic energy generator, etc. The energy source 523 can be coupled to the leaflet attachment device 500 by a line 525 (e.g., cable, wire, cord, etc.) configured to transmit energy from the energy source 523 to the leaflet attachment device 500 (e.g., to the contact surfaces of the leaflet attachment device). In some implementations, the line 525 can extend from the energy source 523 to the leaflet attachment device 500 through the delivery system 502.

FIG. 60 illustrates an example approach for attaching two native heart valve leaflets (e.g., valve leaflets 20, 22) together. In the example implementation of FIG. 60, the leaflets are attached together by a bio-compatible adhesive 798, in addition to fusing the leaflets together with energy or instead of fusing the leaflets together with energy.

The bio-compatible adhesive 798 can be configured in a variety of ways. For example, the type of adhesive used can be any suitable bio-compatible adhesive capable of forming a sufficient bond to attach and hold two native heart valve leaflets together while the heart is pumping. Suitable bio-compatible adhesives include, but are not limited to, Bogle® from Artivion or Dermabond® from Ethicon. The adhesive 798 can be delivered in a variety of ways. For example, in some implementations, the adhesive 798 is applied between to captures leaflets without any additional structure (i.e., only the adhesive is positioned between the two valves leaflets). In other implementations, the adhesive 798 can be applied onto other structure that is positioned between the leaflets.

In some implementations, the adhesive is positioned on an exterior surface or embedded into a coaptation element (e.g., graft, spacer, coaption element, gap filler, membrane, sheet, plug, wedge, balloon, etc.) that is positioned between the heart valves. In some implementations, the adhesive is positioned on a graft or substrate that is positioned between the heart valves. In some implementations, the coaptation element and/or graft/substrate includes additional attachment-enhancing features, such as barbs, hooks, folds, ridges, extensions, loops, etc.

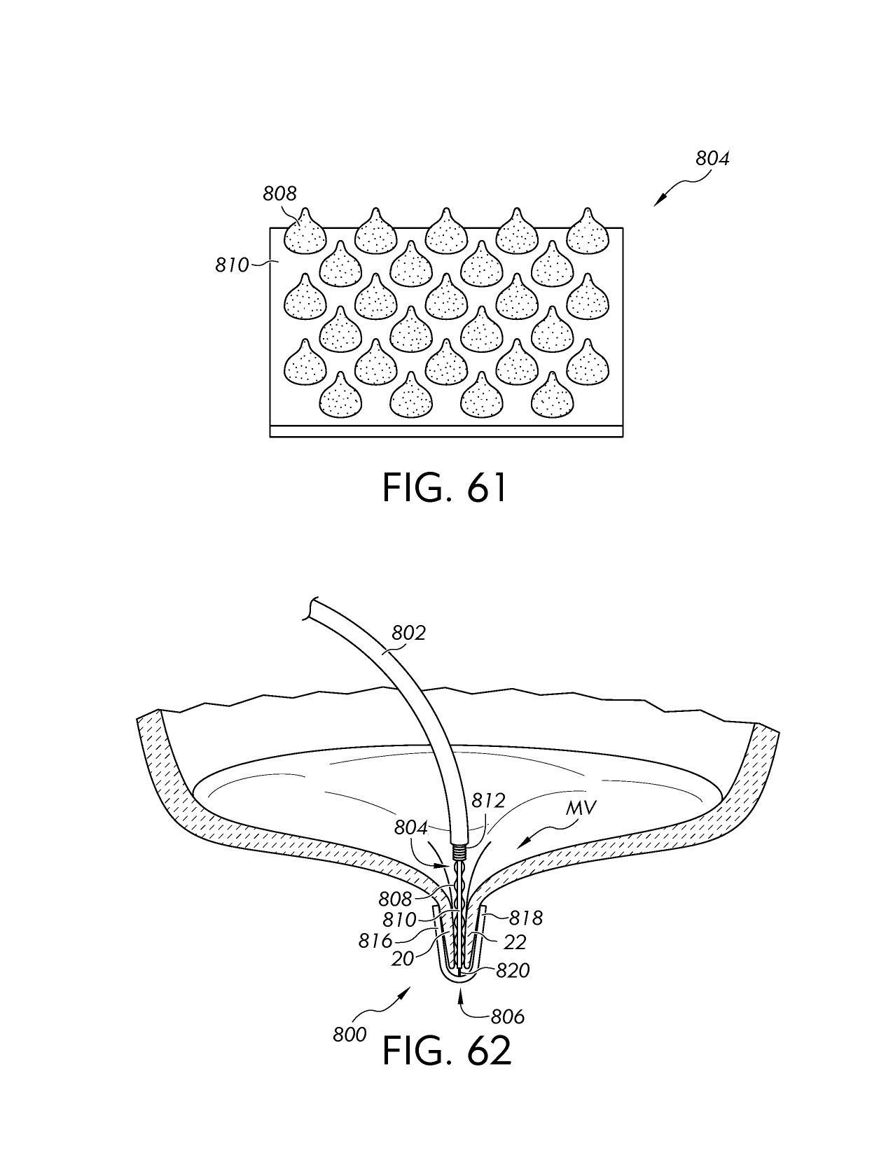

FIGS. 61-62 schematically illustrate an example leaflet attachment device 800. The leaflet attachment device 800 is configured to be delivered to the location of a native heart valve (e.g., mitral valve, tricuspid valve). The leaflet attachment device 800 can be configured to be delivered via a delivery system 802 or other means for delivery. The delivery system 802 can be any suitable delivery system. For example, the delivery system 802 can comprise one or more of a catheter, a sheath, a guide catheter/sheath, a delivery catheter/sheath, a steerable catheter, an implant catheter, a tube, a channel, a pathway, combinations of these, etc. In some implementations, at least a portion of the device 800 is disposed at the end of a catheter of the delivery system 802.

Referring to FIG. 62, in the illustrated example, the device 800 includes a coaptation element 804 (e.g., graft, implant, spacer, coaption element, gap filler, membrane, sheet, plug, wedge, balloon, etc.) and leaflet pinching mechanism 806. In some implementations, the coaptation element 804 is configured to be implanted between leaflets (e.g., leaflets 20, 22) of a native heart valve. In some implementations, the coaptation element 804 is disposed at the end of a catheter of the delivery system 802 by a releasable coupler 812. Thus, the coaptation element 804 can be released and implanted between leaflets 20, 22.

The coaptation element 804 can be configured in a variety of ways. Referring to FIG. 61, in some implementations, the coaptation element 804 is configured to house (e.g., embedded with) an adhesive 808. For example, the coaptation element 804 can include body 810 made of a spongey material with the adhesive embedded in the spongey body. In the illustrated examples, the body 810 is configured as a rectangular sheet. In other implementation, the body 810 can be any suitable shape that can be positioned between leaflets to bond the leaflets together. In some implementations, the coaptation element 804 and/or body 810 includes additional attachment-enhancing features, such as barbs, hooks, folds, ridges, extensions, loops, etc.

In some implementations, the leaflet pinching mechanism 806 is configured to engage the leaflets 20, 22 and pinch the coaptation element 804 between the leaflets 20, 22. The leaflet pinching mechanism 806 can be configured in a variety of ways. For example, the leaflet pinching mechanism 806 can be any device disclosed herein that can pinch leaflets together or against the coaptation element 804. In some implementations, the leaflet pinching mechanism 806 is a separate mechanism used in conjunction with the coaptation element 804.

In some implementations, the leaflet pinching mechanism can be configured to detach from the delivery system and stay with the adhesive and the coaptation element or the leaflet pinching mechanism can be configured to be removed from the native valve by the delivery system and leave only the adhesive and/or the coaptation element in the native valve.

In the illustrated example, the leaflet pinching mechanism 806 includes a first arm 816 and a second arm 818 that are movable between an open condition and a closed condition. The first arm 816 and a second arm 818 can be moved between the open condition and the closed condition by any suitable means. In some implementations, an actuation element 820 (e.g., actuation wire, shaft, tube, hypotube, line, suture, braid, etc.) can be provided to move the arms 816, 818 between the open condition and the closed condition.

As shown in FIG. 62, to attach the leaflets 20, 22 together the leaflet pinching mechanism 806 is positioned at the native valve. For example, in the illustrated example, the leaflet pinching mechanism 806 is positioned below the mitral valve MV such that the first arm 816 and the second arm 818 are positioned outside of the leaflets 20, 22. In some implementations, the coaptation element 804 is positioned between the leaflets 20, 22 such that when the leaflet pinching mechanism 806 is moved to the closed condition, the arms 816, 818 pinch the leaflets 20, 22 against the coaptation element 804.

In some implementations, when the coaptation element 804 is squeezed between the leaflets 20, 22, the adhesive 808 in the adhesive-infused body 810 becomes exposed and activated.

In some implementations, the leaflet pinching mechanism 806 can be kept in place a sufficient time to allow the adhesive to cure to bond the leaflets together. In some implementations, once the adhesive is cured, the coaptation element 804 can be released from the delivery system and will remain in place and the leaflet pinching mechanism 806 can be withdrawn from the valve (e.g., through an unbonded portion of the valve).

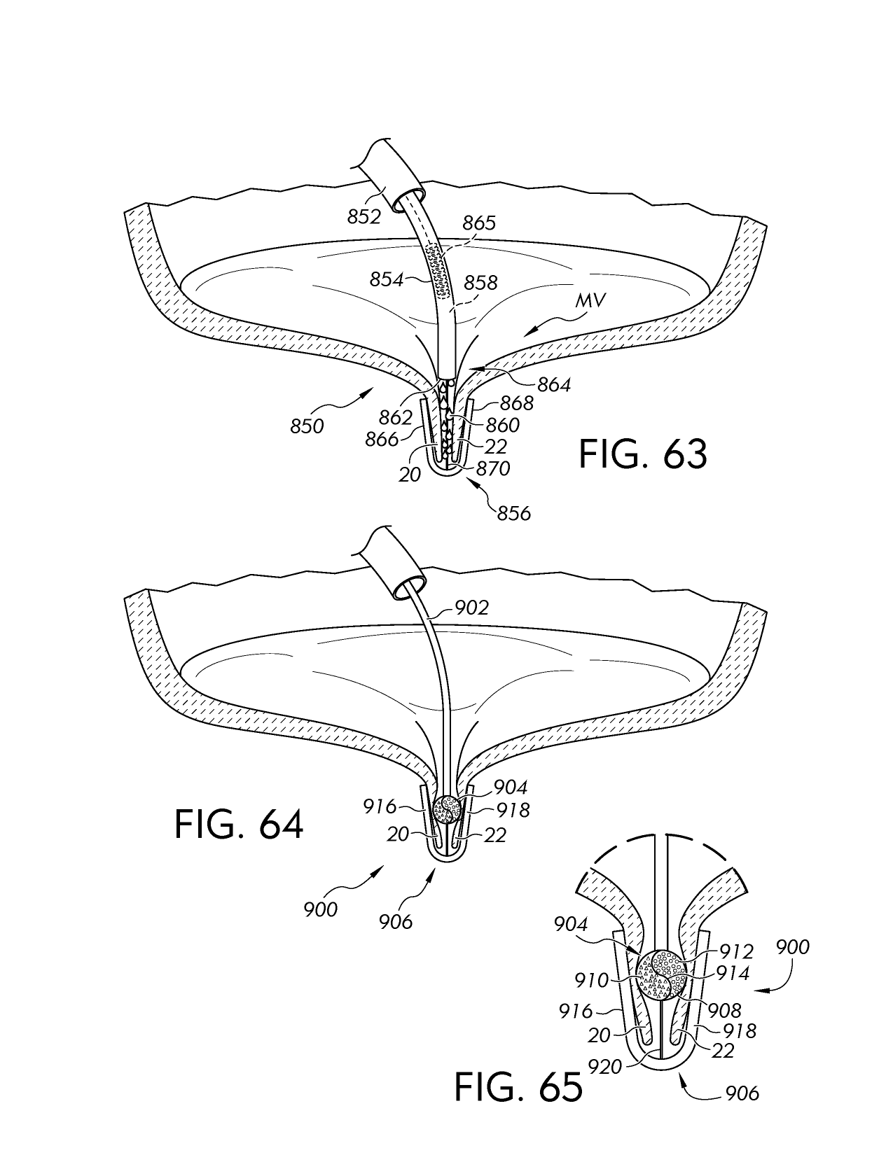

FIG. 63 schematically illustrates an example leaflet attachment device 850. The leaflet attachment device 850 is configured to be delivered to the location of a native heart valve (e.g., mitral valve, tricuspid valve). The leaflet attachment device 850 can be configured to be delivered via a delivery system 852 or other means for delivery. The delivery system 852 can be any suitable delivery system. For example, the delivery system 852 can comprise one or more of a catheter, a sheath, a guide catheter/sheath, a delivery catheter/sheath, a steerable catheter, an implant catheter, a tube, a channel, a pathway, combinations of these, etc.

In illustrated example, the device 850 includes an adhesive delivery conduit 854 and leaflet pinching mechanism 856. In some implementations, the adhesive delivery conduit 854 includes an internal passage 858 configured to deliver adhesive 860 therethrough. The adhesive delivery conduit 854 can include an adhesive outlet 862 at a distal end 864 of the adhesive delivery conduit 854. In some implementations, a static mixer 865 can be positioned in the internal passage 858. Where the adhesive 860 is a two-part adhesive or multi-part adhesive, the static mixer 865 can be configured to mix the adhesive 860 prior to the adhesive 860 exiting the adhesive outlet 862. Any suitable static mixer configuration can be used.

In some implementations, the leaflet pinching mechanism 856 is configured to engage the leaflets 20, 22 and pinch the leaflets 20, 22 together. The leaflet pinching mechanism 856 can be configured in a variety of ways. For example, the leaflet pinching mechanism 856 can be any device disclosed herein that can pinch leaflets together. In some implementations, the leaflet pinching mechanism 856 is a separate mechanism used in conjunction with adhesive delivery conduit 854.

In the illustrated example, the leaflet pinching mechanism 856 includes a first surface or a first arm 866 and a second surface or a second arm 868 that are movable between an open condition and a closed condition. The first surface or first arm 866 and second surface or second arm 868 can be moved between the open condition and the closed condition by any suitable means. In some implementations, an actuation element 870 (e.g., actuation wire, shaft, tube, hypotube, line, suture, braid, etc.) can be provided to move the surfaces or arms 866, 868 between the open condition and the closed condition.

To attach the leaflets 20, 22 together, the leaflet pinching mechanism 856 is positioned at the native valve. For example, in the illustrated example, the leaflet pinching mechanism 856 is positioned below the mitral valve MV such that the first arm 866 and the second arm 868 are positioned outside of the leaflets 20, 22.

In some implementations, the adhesive delivery conduit 854 is positioned such that adhesive 860 exiting the adhesive outlet 862 is introduced between the leaflets 20, 22. In some implementations, the leaflet pinching mechanism 856 can be kept in place a sufficient time to allow the adhesive 860 to cure and bond the leaflets 20, 22 together. Once the adhesive 860 is cured, the leaflet pinching mechanism 806 can be withdrawn from the valve (e.g., through an unbonded portion of the valve).

In some implementations, the leaflet pinching mechanism 806 can be detached from the delivery system and left with the native valve with the adhesive.

In some implementations, the adhesive can be used in conjunction with heat fusing of the leaflets to one another or to a coaptation element.

FIGS. 64-65 schematically illustrate an example leaflet attachment device 900. The leaflet attachment device 900 is configured to be delivered to the location of a native heart valve (e.g., mitral valve, tricuspid valve). The leaflet attachment device 900 can be configured to be delivered via a delivery system 902 or other means for delivery. The delivery system 902 can be any suitable delivery system. For example, the delivery system 902 can comprise one or more of a catheter, a sheath, a guide catheter/sheath, a delivery catheter/sheath, a steerable catheter, an implant catheter, a tube, a channel, a pathway, combinations of these, etc.

In illustrated example, the device 900 includes an encapsulated adhesive 904 and leaflet pinching mechanism 906. The encapsulated adhesive 904 can be configured in a variety of ways. Any suitable configuration that allows the adhesive 904 to be delivered to a native valve (e.g., the mitral valve MV) via the distal end of a catheter and then activated once in position can be used. In some implementations, the adhesive includes a first component and a second component, at least one of the first component and second component being encapsulated in a manner that prevents or inhibits its interaction with the other component until de-encapsulated. In some implementations, the adhesive 904 can be any of the multi-component compositions disclosed in U.S. Provisional Patent Application Ser. No. 63/497,437, entitled Multi-Component Composition and Uses Thereof, filed April 20, 2023, the entire disclosure of which is incorporated herein by reference.

In some implementations, the encapsulated adhesive 904 includes an adhesive within an encapsulating structure 908 (e.g., a casing, a matrix, a lattice, a cavity, a balloon, an implant, a capsule, etc.). In some implementations, the adhesive 904 is a multi-part composition having a first component 910 separated from a second component 912 by a barrier 914 (FIG. 65) (e.g., one or both of the first component or the second component are encapsulated). In some implementations, the adhesive composition can include two component or can include more than two components. In some implementations, the encapsulated adhesive 904 is disposed at a distal end of a catheter of the delivery system 902.

In some implementations, the leaflet pinching mechanism 906 is configured to engage the leaflets 20, 22 and pinch the leaflets 20, 22 together. The leaflet pinching mechanism 906 can be configured in a variety of ways. For example, the leaflet pinching mechanism 906 can be any device disclosed herein that can pinch leaflets together. In some implementations, the leaflet pinching mechanism 906 is a separate mechanism used in conjunction with the catheter used to deliver the encapsulated adhesive 904.

In the illustrated example, the leaflet pinching mechanism 906 includes a first arm 916 and a second arm 918 that are movable between an open condition and a closed condition. The first arm 916 and a second arm 918 can be moved between the open condition and the closed condition by any suitable means. In some implementations, an actuation element 920 (e.g., actuation wire, shaft, tube, hypotube, line, suture, braid, etc.) can be provided to move the arms 866, 868 between the open condition and the closed condition.

To attach the leaflets 20, 22 together, the leaflet pinching mechanism 906 is positioned at the native valve. For example, in the illustrated example, the leaflet pinching mechanism 906 is positioned below the mitral valve MV such that the first arm 916 and the second arm 918 are positioned outside of the leaflets 20, 22.

In some implementations, encapsulated adhesive 904 is positioned between the leaflets 20, 22. In some implementations, when the surfaces or arms 916, 918 are moved to the closed condition, the leaflet pinching mechanism 906 squeezes the encapsulated adhesive 904 and bursts the encapsulating structure 908 such that the first part 910 and the second part 912 of the adhesive 904 mix and are activated. In some implementations, the encapsulating structure 908 can be made of a dissolvable material such that the encapsulating structure 908 dissolves rather than bursts. In some implementations, the encapsulating structure 908 includes a porous material (e.g., cloth) such that the adhesive 904 will pass through the encapsulating structure 908 (e.g., in time or in response to pressure applied by the leaflet pinching mechanism. The leaflet pinching mechanism 906 can be kept in place a sufficient time to allow the adhesive 904 to cure and bond the leaflets 20, 22 together. Once the adhesive 904 is cured, the leaflet pinching mechanism 906 can be withdrawn from the valve (e.g., through an unbonded portion of the valve).

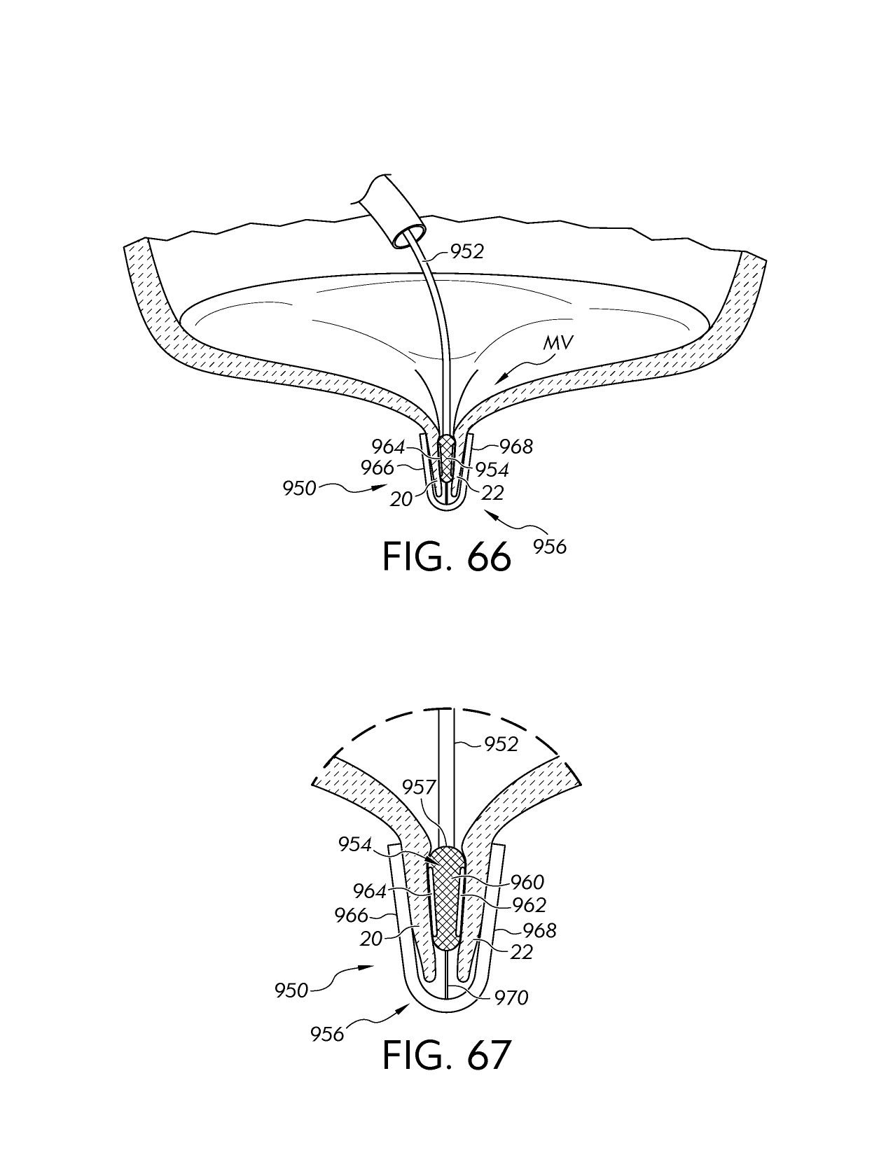

FIGS. 66-67 schematically illustrate an example leaflet attachment device 950. The leaflet attachment device 950 is configured to be delivered to the location of a native heart valve (e.g., mitral valve, tricuspid valve). The leaflet attachment device 950 can be configured to be delivered via a delivery system 952 or other means for delivery. The delivery system 952 can be any suitable delivery system. For example, the delivery system 952 can comprise one or more of a catheter, a sheath, a guide catheter/sheath, a delivery catheter/sheath, a steerable catheter, an implant catheter, a tube, a channel, a pathway, combinations of these, etc. In some implementations, at least a portion of the device 950 is disposed at the end of a catheter of the delivery system 952.

In the illustrated example, the device 950 includes a coaptation element 954 (e.g., graft, implant, spacer, coaption element, gap filler, membrane, sheet, plug, wedge, balloon, etc.) and leaflet pinching mechanism 956. In some implementations, the coaptation element 954 is configured to be implanted between leaflets (e.g., leaflets 20, 22) of a native heart valve. In some implementations, the coaptation element 954 is disposed at the end of a catheter of the delivery system 952 by a releasable coupler 957. Thus, the coaptation element 954 can be released and implanted between leaflets 20, 22.

The coaptation element 954 can be configured in a variety of ways. In some implementations, the coaptation element 954 includes a body 960 having an exterior surface 962. In some implementations, an adhesive 964 can be applied to the exterior surface 962 either prior to delivery or once in place within the valve. In some implementations, the adhesive 964 is disposed on or embedded into a body or substrate that is attached to the coaptation element 954.

In some implementations, the leaflet pinching mechanism 956 is configured to engage the leaflets 20, 22 and pinch the coaptation element 954 between the leaflets 20, 22. The leaflet pinching mechanism 956 can be configured in a variety of ways. For example, the leaflet pinching mechanism 956 can be any device disclosed herein that can pinch leaflets together or against the coaptation element 954. In some implementations, the leaflet pinching mechanism 956 is a separate mechanism used in conjunction with the coaptation element 954.

In the illustrated example, the leaflet pinching mechanism 956 includes a first arm 966 and a second arm 968 that are movable between an open condition and a closed condition. The first arm 966 and a second arm 968 can be moved between the open condition and the closed condition by any suitable means. In some implementations, an actuation element 970 (e.g., actuation wire, shaft, tube, hypotube, line, suture, braid, etc.) can be provided to move the arms 966, 968 between the open condition and the closed condition.

To attach the leaflets 20, 22 together, the leaflet pinching mechanism 956 is positioned at the native valve. For example, in the illustrated example, the leaflet pinching mechanism 956 is positioned below the mitral valve MV such that the first arm 966 and the second arm 968 are positioned outside of the leaflets 20, 22.

In some implementations, coaptation element 954 is positioned between the leaflets 20, 22 such that when the leaflet pinching mechanism 956 is moved to the closed condition, the surfaces or arms 966, 968 pinch the leaflets 20, 22 together and/or against a coaptation element 954.

In some implementations, when the coaptation element 954 is squeezed between the leaflets 20, 22, the adhesive 964 bonds the leaflets 20, 22 to the coaptation element 954. In some implementations, the leaflet pinching mechanism 956 is configured to move the first surface or first arm 966 and the second surface or second arm 968 independent of each other such a first of the leaflets can be captured and adhered to the coaptation element 954 prior to the second of the leaflets. Thus, the device 950 can be moved into positioned to capture the second leaflet while the first leaflet is already captured. The leaflet pinching mechanism 956 can be kept in place a sufficient time to allow the adhesive to cure to bond the leaflets together. Once the adhesive is cured, the coaptation element 954 can be released from the delivery system 952 and will remain in place and the leaflet pinching mechanism 956 can be withdrawn from the valve (e.g., through an unbonded portion of the valve).

FIGS. 66-67 schematically illustrate an example leaflet attachment device 950. The leaflet attachment device 950 is configured to be delivered to the location of a native heart valve (e.g., mitral valve, tricuspid valve). The leaflet attachment device 950 can be configured to be delivered via a delivery system 952 or other means for delivery. The delivery system 952 can be any suitable delivery system. For example, the delivery system 952 can comprise one or more of a catheter, a sheath, a guide catheter/sheath, a delivery catheter/sheath, a steerable catheter, an implant catheter, a tube, a channel, a pathway, combinations of these, etc. In some implementations, at least a portion of the device 950 is disposed at the end of a catheter of the delivery system 952.

In the illustrated example, the device 950 includes a coaptation element 954 (e.g., graft, implant, spacer, coaption element, gap filler, membrane, sheet, plug, wedge, balloon, etc.) and leaflet pinching mechanism 956. In some implementations, the coaptation element 954 is configured to be implanted between leaflets (e.g., leaflets 20, 22) of a native heart valve. In some implementations, the coaptation element 954 is disposed at the end of a catheter of the delivery system 952 by a releasable coupler 957. Thus, the coaptation element 954 can be released and implanted between leaflets 20, 22.

The coaptation element 954 can be configured in a variety of ways. In some implementations, the coaptation element 954 includes a body 960 having an exterior surface 962. An adhesive 964 can be applied to the exterior surface 962 either prior to delivery or once the coaptation element 954 is positioned within the valve. In some implementations, the coaptation element 954 can be biodegradable.

The leaflet pinching mechanism 956 is configured to engage the leaflets 20, 22 and pinch the coaptation element 954 between the leaflets 20, 22. The leaflet pinching mechanism 956 can be configured in a variety of ways. For example, the leaflet pinching mechanism 956 can be any device disclosed herein that can pinch leaflets together or against the coaptation element 954. In some implementations, the leaflet pinching mechanism 956 is a separate mechanism used in conjunction with the coaptation element 954.

In the illustrated example, the leaflet pinching mechanism 956 includes a first arm 966 and a second arm 968 that are movable between an open condition and a closed condition. The first arm 966 and a second arm 968 can be moved between the open condition and the closed condition by any suitable means. In some implementations, an actuation element 970 (e.g., actuation wire, shaft, tube, hypotube, line, suture, braid, etc.) can be provided to move the arms 966. 968 between the open condition and the closed condition.

To attach the leaflets 20, 22 together, the leaflet pinching mechanism 956 is positioned at the native valve. For example, in the illustrated example, the leaflet pinching mechanism 956 is positioned below the mitral valve MV such that the first surface or first arm 966 and the second surface or second arm 968 are positioned outside of the leaflets 20, 22. T

In some implementations, the coaptation element 954 is positioned between the leaflets 20, 22 such that when the leaflet pinching mechanism 956 is moved to the closed condition, the arms 966, 968 pinch the leaflets 20, 22 together and/or against the coaptation element 954. In some implementations, when the coaptation element 954 is squeezed between the leaflets 20, 22, the adhesive 964 bonds the leaflets 20, 22 to the coaptation element 954. In some implementations, the leaflet pinching mechanism 956 is configured to move the first surface or first arm 966 and the second surface or second arm 968 independent of each other such a first of the leaflets can be captured and adhered to the coaptation element 954 prior to the second of the leaflets. Thus, the device 950 can be moved into positioned to capture the second leaflet while the first leaflet is already captured. The leaflet pinching mechanism 956 can be kept in place a sufficient time to allow the adhesive to cure to bond the leaflets together. Once the adhesive is cured, the coaptation element 954 can be released from the delivery system 952 and will remain in place and the leaflet pinching mechanism 956 can be withdrawn from the valve (e.g., through an unbonded portion of the valve).

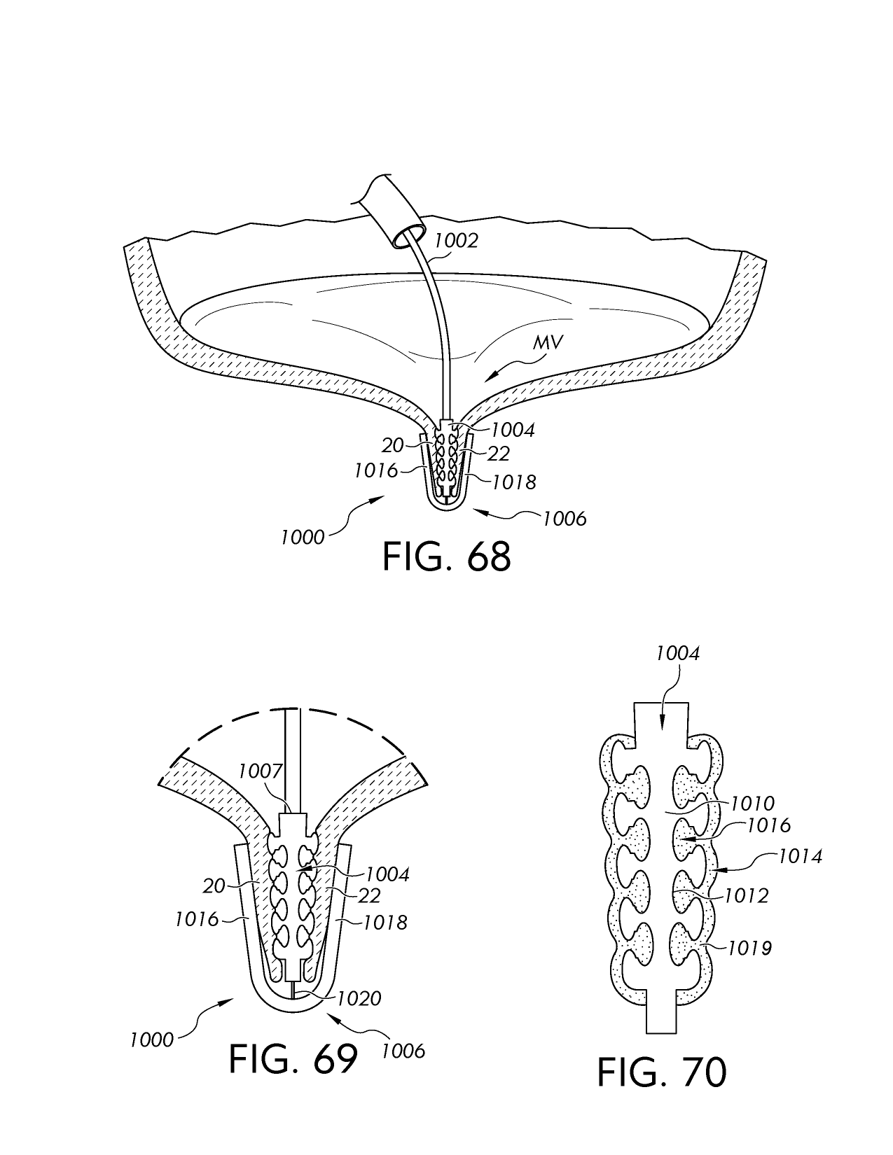

FIGS. 68-70 schematically illustrate an example leaflet attachment device 1000. The leaflet attachment device 1000 is configured to be delivered to the location of a native heart valve (e.g., mitral valve, tricuspid valve). The leaflet attachment device 1000 can be configured to be delivered via a delivery system 1002 or other means for delivery. The delivery system 1002 can be any suitable delivery system. For example, the delivery system 1002 can comprise one or more of a catheter, a sheath, a guide catheter/sheath, a delivery catheter/sheath, a steerable catheter, an implant catheter, a tube, a channel, a pathway, combinations of these, etc. In some implementations, at least a portion of the device 1000 is disposed at the end of a catheter of the delivery system 1002.

In the illustrated example, the device 1000 includes a coaptation element 1004 (e.g., graft, implant, spacer, coaption element, gap filler, membrane, sheet, plug, wedge, balloon, etc.) and a leaflet pinching mechanism 1006. In some implementations, the coaptation element 1004 is configured to be implanted between leaflets (e.g., leaflets 20, 22) of a native heart valve. In some implementations, the coaptation element 1004 is disposed at the end of a catheter of the delivery system 1002 by a releasable coupler 1007. Thus, the coaptation element 1004 can be released and implanted between leaflets 20, 22.

The coaptation element 1004 can be configured in a variety of ways. In some implementations, the coaptation element 1004 includes a body 1010 having an exterior surface 1012 configured to increase the overall exterior surface area. For example, in the illustrated example, the exterior surface 1012 includes a plurality of peaks or projections 1014 and valleys or recesses 1016. In some implementations, an adhesive 1019 can be applied to the exterior surface 1012 either prior to delivery or once the coaptation element 1004 is positioned within the valve. In some implementations, the coaptation element 1004 can be biodegradable.

In some implementations, the leaflet pinching mechanism 1006 is configured to engage the leaflets 20, 22 and pinch the coaptation element 1004 between the leaflets 20, 22. The leaflet pinching mechanism 1006 can be configured in a variety of ways. For example, the leaflet pinching mechanism 1006 can be any device disclosed herein that can pinch leaflets together or against the coaptation element 1004. In some implementations, the leaflet pinching mechanism 1006 is a separate mechanism used in conjunction with the coaptation element 1004.

In the illustrated example, the leaflet pinching mechanism 1006 includes a first arm 1016 and a second arm 1018 that are movable between an open condition and a closed condition. The first arm 1016 and a second arm 1018 can be moved between the open condition and the closed condition by any suitable means. In some implementations, an actuation element 1020 (e.g., actuation wire, shaft, tube, hypotube, line, suture, braid, etc.) can be provided to move the arms 1016, 1018 between the open condition and the closed condition.

To attach the leaflets 20, 22 together, the leaflet pinching mechanism 1006 is positioned at the native valve. For example, in the illustrated example, the leaflet pinching mechanism 1006 is positioned below the mitral valve MV such that the first arm 1016 and the second arm 1018 are positioned outside of the leaflets 20, 22.

In some implementations, the coaptation element 1004 is positioned between the leaflets 20, 22 such that when the leaflet pinching mechanism 1016 is moved to the closed condition, the arms 1016, 1018 pinch the leaflets 20, 22 against the coaptation element 1004. In some implementations, when the coaptation element 1004 is squeezed between the leaflets 20, 22, the adhesive 1019 bonds the leaflets 20, 22 to the coaptation element 1004. In some implementations, the leaflet pinching mechanism 1016 is configured to move the first arm 1016 and the second arm 1018 independent of each other such a first of the leaflets can be captured and adhered to the coaptation element 1004 prior to the second of the leaflets. Thus, the device 1000 can be moved into positioned to capture the second leaflet while the first leaflet is already captured. The leaflet pinching mechanism 1006 can be kept in place a sufficient time to allow the adhesive to cure to bond the leaflets together. Once the adhesive is cured, the coaptation element 1004 can be released from the delivery system 1002 and will remain in place and the leaflet pinching mechanism 1006 can be withdrawn from the valve (e.g., through an unbonded portion of the valve).

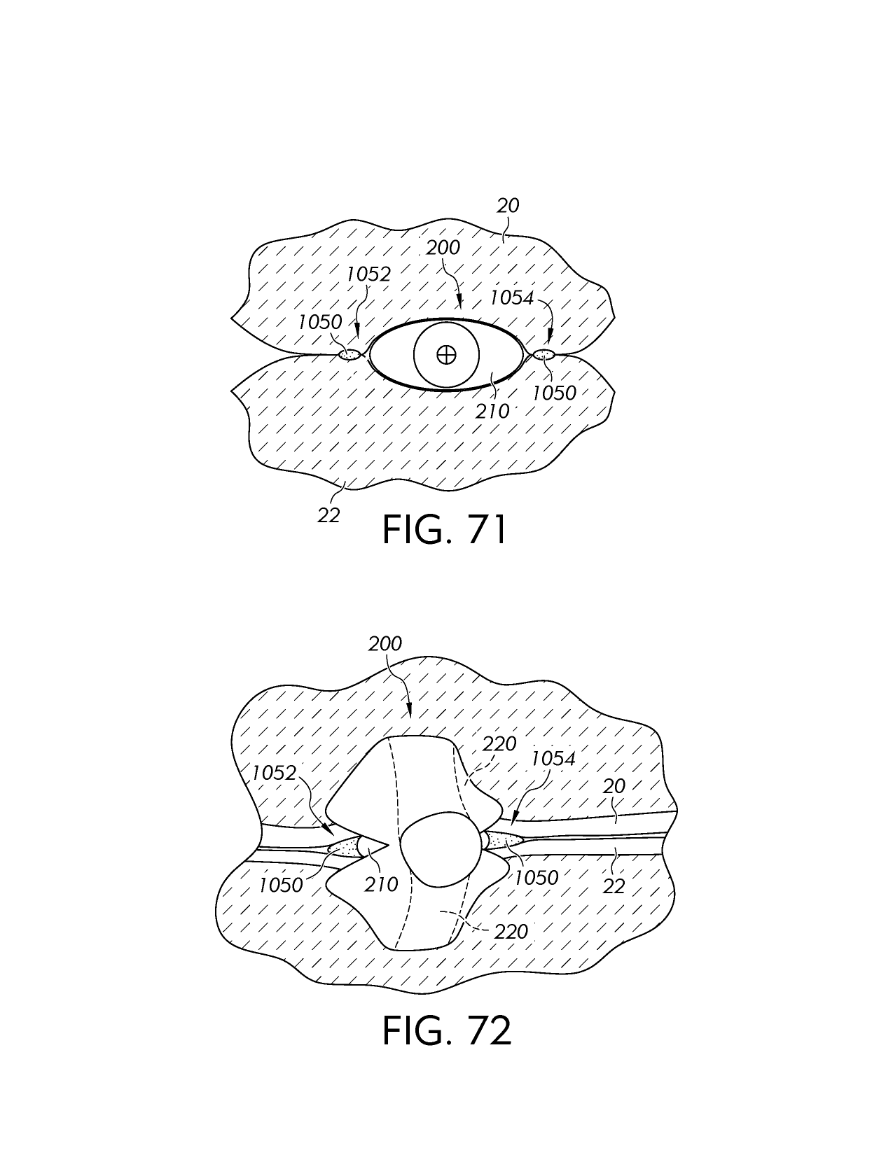

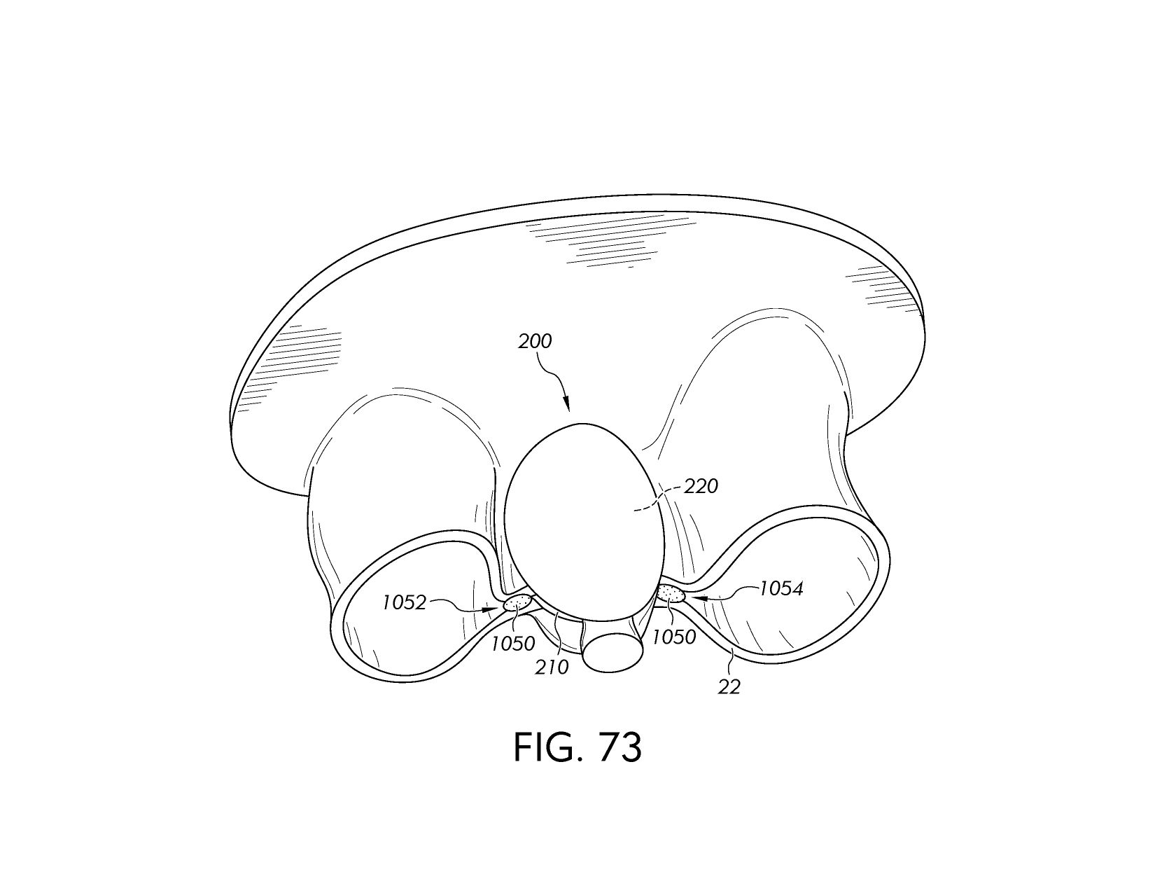

Referring to FIGS. 71-73, an example of an implantable device or implant is shown used in conjunction with adhesive and/or fusion to repair a native heart valve. The device can include any other features for any of the devices or implants disclosed herein. The example of FIGS. 71-73 illustrates the device 200 of FIG. 22 deployed in the mitral valve MV with the leaflets 20, 22 captured. In particular, the outer paddles 220 are illustrated in the closed condition with the leaflets 20, 22 pinched between the outer paddles 220 and the coaptation element 210.

In addition to the implantable device or implant 200, the first leaflet 20 can also be bonded to the second leaflet 22 by adhesive 1050 in one or more locations (e.g., at or near a gap between the leaflets adjacent to the coaptation element). In some implementations, the first leaflet 20 and the second leaflet 22 are bonded by adhesive at a first location 1052 and at a second location 1054. In the illustrated example, the first location 1052 is lateral to and adjacent the device 200 and the second location 1054 is lateral to and adjacent the device 200 opposite the first location 1052.

The adhesive 1050 can be applied in any suitable manner, such as for example, by any device disclosed herein. In some implementations, the implantable device or implant 200 can be left implanted in the native valve or can be removed once the adhesive 1050 has cured. In the illustrated examples, the area of the native valve between the first location 1052 and the second location 1054 is unbonded. Thus, the implantable device or implant 200 can be removed through the unbonded portion should removing the device or implant 200 be desired.

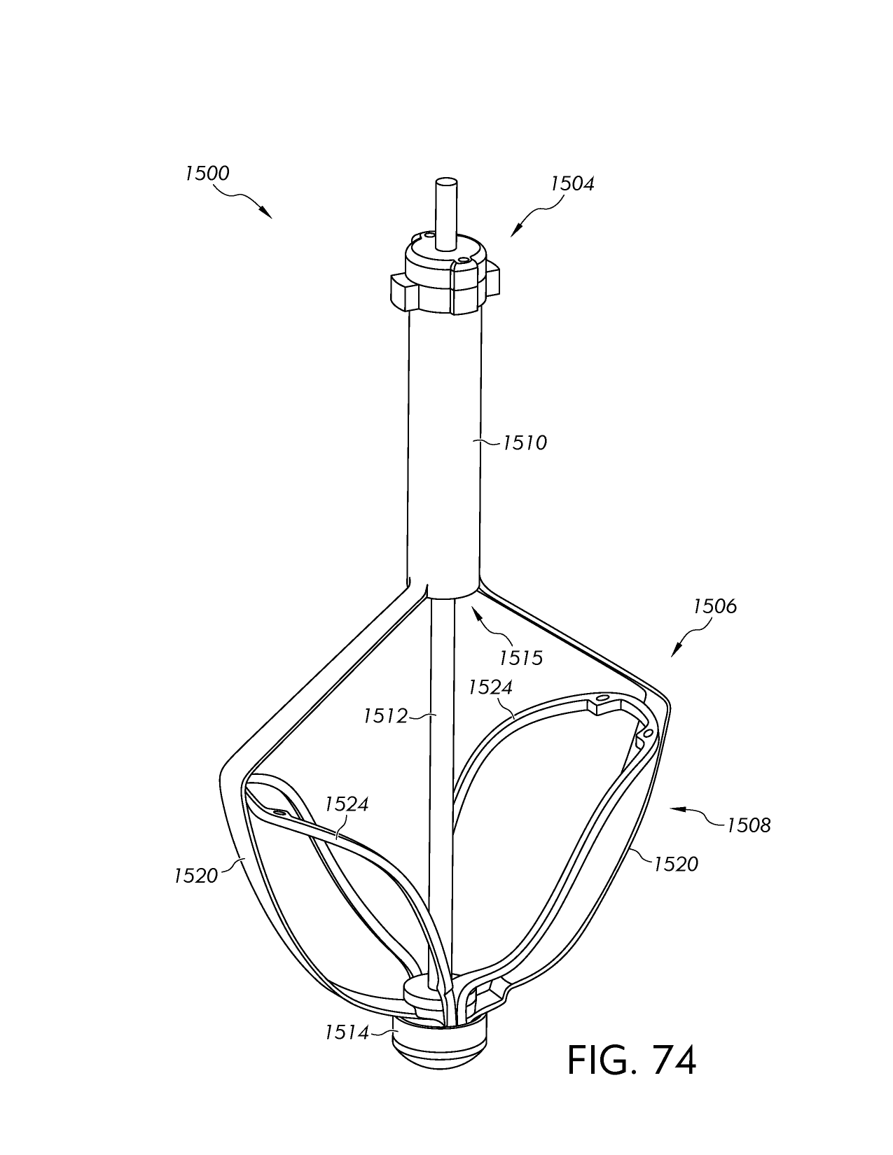

FIG. 74 illustrates an example implantable device or implant 1500. The device 1500 is configured to be positioned to engage leaflets of 20, 22 as part of any suitable valve repair system (e.g., any valve repair system disclosed in the present application). The device 1500 can include any of the features for an implantable device or implant discussed in the present application. The device 1500 can move between an elongated or fully open condition (e.g., device 100 in FIG. 8) and a fully closed condition (e.g., device 100 in FIG. 14). In the illustrated example of FIG. 74, the device 1500 is in a partially closed, capture ready condition.

In some implementations, the implantable device or implant 1500 includes a coaptation portion 1504 and an anchor portion 1506. In some implementations, the coaptation portion 1504 of the device 1500 optionally includes a coaptation element 1510 (e.g., spacer, coaptation element, gap filler, membrane, sheet, plug, wedge, balloon, etc.) for implantation between leaflets of a native valve. In some implementations, the anchor portion 1506 includes a plurality of anchors 1508. In some implementations, each anchor 1508 includes arms or paddles 1520 and paddle extension members or paddle frames 1524. In some implementations, the arms or paddles 1520 are attached at one end to a distal portion 1515 of the coaptation element 1510 and at the other end to an end cap 1514.

In some implementations, the coaptation element 1510 and paddles 1520 are formed from a flexible material that can be collapsible wires, a metal fabric, such as a mesh, woven, braided, or formed in any other suitable way or a laser cut or otherwise cut flexible material. The material can be shape memory wires—such as Nitinol—to provide shape-setting capability, or any other flexible material suitable for implantation in the human body.

In some implementations, the paddles 1520 are made from wires such that the paddles 1520 can be readily collapsed into the elongated condition. As shown in FIG. 74, the paddles 1520 can be narrowly configured such that the device 1500 presents a narrower profile (e.g., width). The narrower width can be advantageous in deployment of the device 1500 and withdrawal of the device 1500 if desired. For example, in some implementations, the device 1500 can be narrowly configured to be withdrawn between the first location 1052 and the second location 1054 while keeping the unbonded portion between the first location 1052 and the second location 1054 narrow.

The paddles 1520 can be moved between the open condition and the closed condition by any suitable means. In some implementations, an actuation element 1512 (e.g., actuation wire, shaft, tube, hypotube, line, suture, braid, etc.) can be provided to move the paddles 1520 between the open condition and the closed condition. In the illustrated example, the actuation element 1512 extends through the coaptation element 1510 and is attached to the end cap 1514 distal to the coaptation element 1510. Movement of the actuation element 1512 distally relative to the coaptation element 1510 moves the device toward the fully open condition and movement of the actuation element 1512 proximally relative to the coaptation element 1510 moves the device toward the fully closed condition.

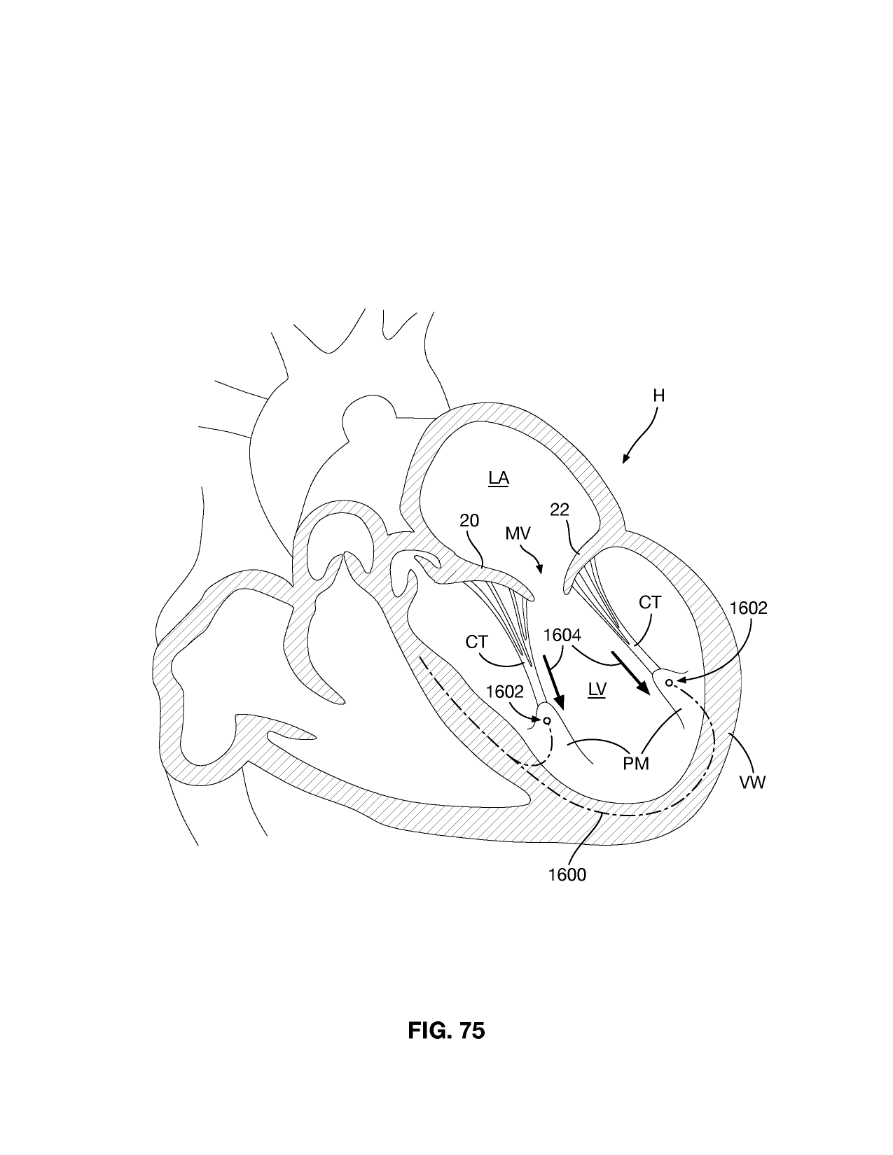

Referring now to FIGS. 75–77, cutaway illustrations of the heart are shown to illustrate an example device and an example procedure for repairing valve regurgitation due to the improper contraction of the ventricle wall caused by, for example, myocardial infarction or dilated cardiomyopathy. A dilated ventricle is larger and can have a thinner and stretched wall such that during systole the ventricle wall does not properly contract. When the ventricle wall does not contract as far as it should and the papillary muscles continue operating properly, the resulting tension applied to the chordae tendineae can hold the leaflets of the mitral or tricuspid valve open, thereby allowing for regurgitation through the valve. The cutaway illustration of the heart in FIG. 75 shows a heart with a dilated left ventricle LV and resulting failure of the mitral valve MV to close. The regurgitation of blood flow through the mitral valve MV into the left atrium LA is caused by the contraction of the papillary muscles PM from a dilated position of the ventricle wall VW so that tension applied to the chordae tendineae CT holds the valve leaflets 20, 22 in an open condition rather than merely prohibiting prolapse of the leaflets 20, 22 into the left atrium LA.

FIG. 75 also illustrates in an abstract or simplified manner a portion of the conductive pathways 1600 through the heart H that cause the contraction of the heart muscle tissue. For example, a signal flows along the illustrated conductive pathways 1600 in the wall of heart H to cause a triggering event 1602 in the papillary muscles PM. Having been triggered, the papillary muscles PM contract and pull the chordae tendineae CT in the direction indicated by the arrows 1604 drawn in FIG. 75. The tension applied to the leaflets 20, 22 by the chordae tendineae CT tethers the leaflets 20, 22, thereby prohibiting or inhibiting the leaflets 20, 22 from proper coaptation.

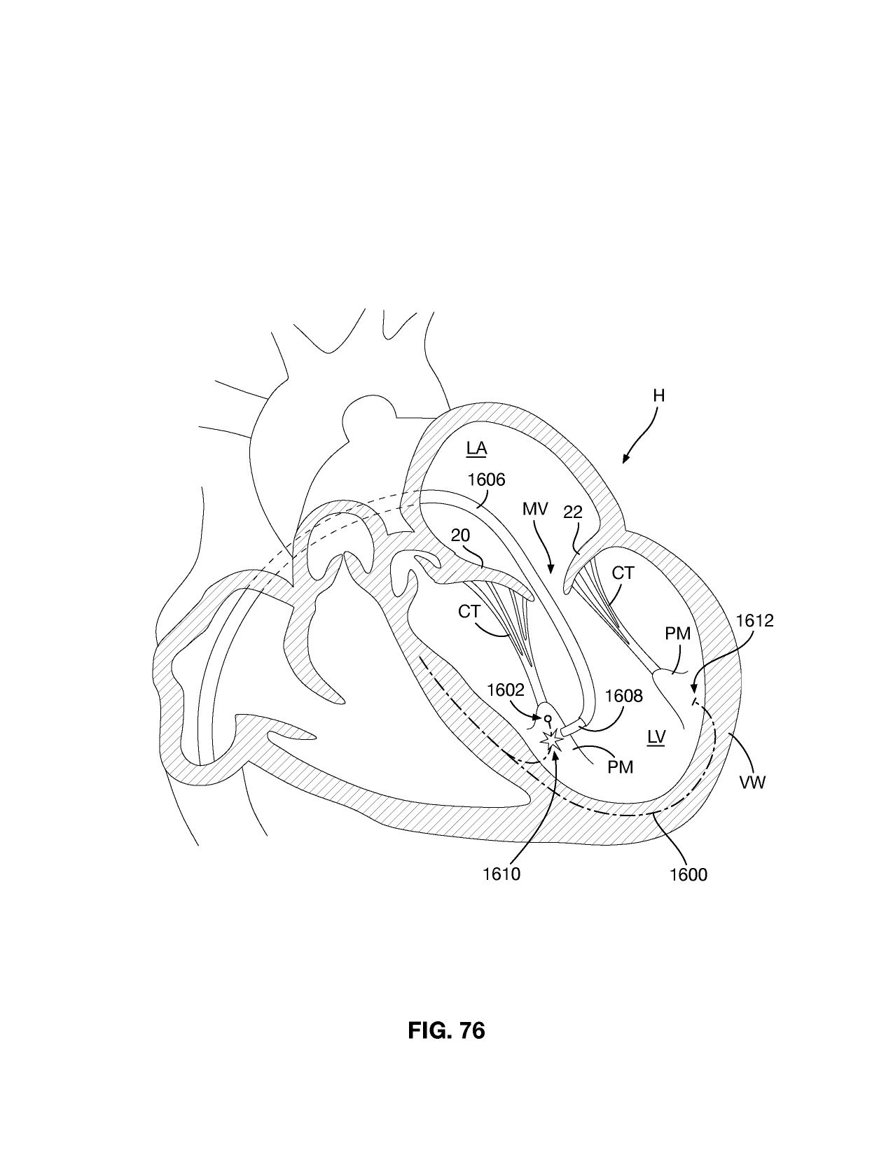

Referring now to FIG. 76, a process is illustrated being performed on a conductive system of the heart. During the example process for relaxing a portion of the heart (e.g., a papillary muscle PM, a portion of heart wall, etc.), a catheter 1606 is used to deliver an ablation device 1608 to a location inside a heart (e.g., proximate a heart wall, proximate a papillary muscle PM, etc.) so that tissue of the heart can be ablated 1610 to disrupt the conductive pathway 1600, e.g., so that the conductive pathway leads to a non-conductive or non-functioning end 1612.

In some implementations, during the example procedure, the catheter 1606 is inserted through a vein and up into the heart. In some implementations, the operator can optionally guide the catheter 1606 through a native valve, e.g., the mitral valve MV, the tricuspid valve TV, the Aortic Valve, the Pulmonic Valve, etc.), and into a corresponding chamber of the heart (e.g., a ventricle of the heart).

In some implementations, a suitable detection device can be used before or during the procedure to monitor the movement of the heart structures and to detect electrical signals transmitted through the conductive pathways 1600. That is, the operator can select one or more portions of the heart (e.g., one or more papillary muscles, one or more regions of the heart wall, etc.) to be relaxed via ablation using the detection device.

In some implementations, an imaging device—separate from or combined with the detection device—can be used to monitor movement of the heart structures and of the catheter 1606 and ablation device 1608.

In some implementations, an example process is performed on the conductive system of a papillary muscle PM. During the example process for relaxing the papillary muscle PM, a catheter 1606 is used to deliver an ablation device 1608 to the papillary muscle PM so that the tissue can be ablated 1610 to disrupt the conductive pathways 1600 and so that the conductive pathway leads to a non-conductive or non-functioning end 1612.

In some implementations, once the catheter 1606 has been delivered to a desired location, the ablation device 1608 is extended from the catheter 1606 to contact the tissue to be relaxed. Energy can be applied to the tissue by the ablation device 1608 by any suitable means, such as, for example, via radio frequency ablation or cryoablation. If a detection device is employed during the procedure, the relaxation of the papillary muscle PM can be confirmed after the ablation device 1608 is used.

In some implementations, the step of ablating the tissue can be repeated multiple times (e.g., 1 time, 2 times, 3 times, 4 times, etc.) in the same or different locations to achieve the desired result. In some implementations, how many ablation steps and/or locations can vary and be optimized based on the geometry of the heart of the patient, e.g., how much the ventricle has dilated, the geometry of the valve leaflets 20, 22, the amount of tension applied to the chordae tendineae CT by the various papillary muscles PM, and the like.

In some implementations, after the tissue of the heart has been ablated to disrupt the conductive pathways 1600, the non-conductive ends 1612 no longer transmit a conductive signal to the tissue, so that the tissue is relaxed during systole and does not contract at all or does not contract to the same degree (e.g., contracts to a reduced or lesser degree). That is, the non-conductive ends 1612 of the conductive pathways 1600 are no longer able to trigger the ablated tissue, so that the ablated tissue cannot be contracted or is contracted less.

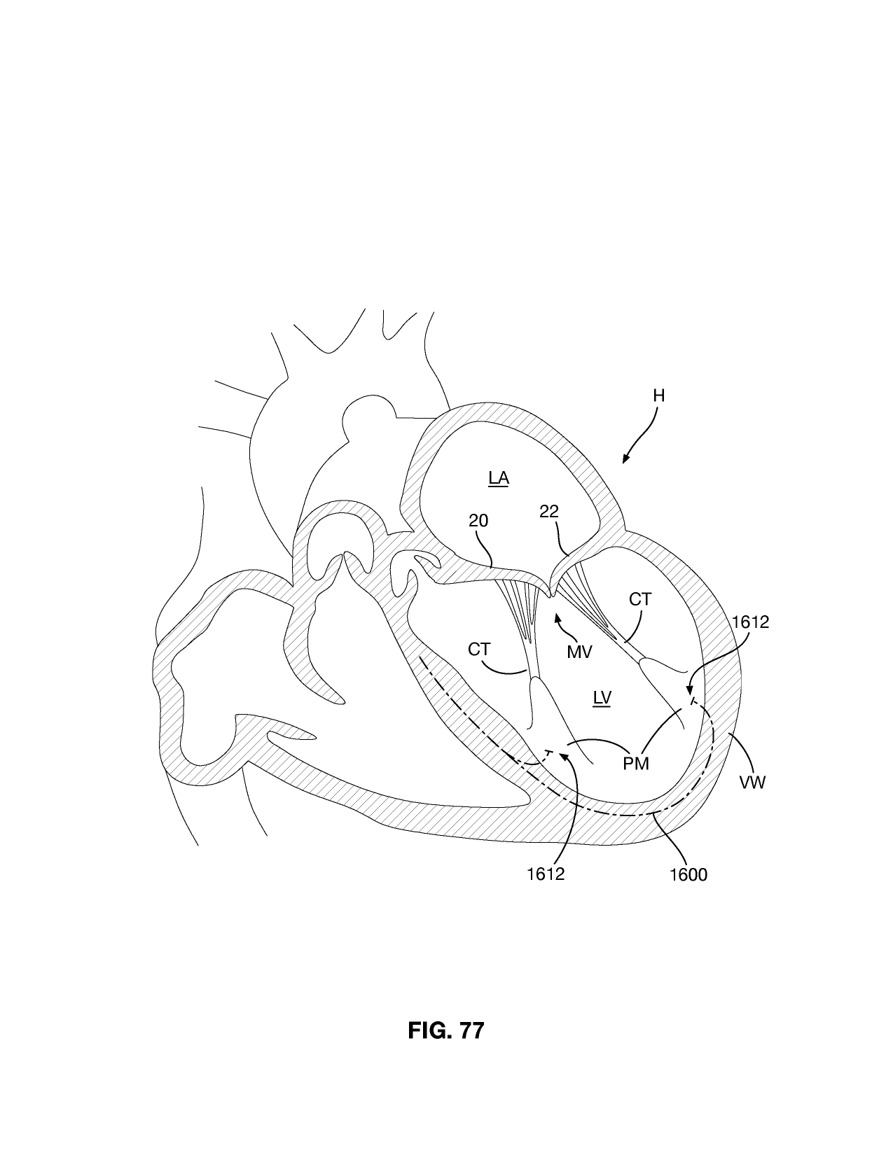

In some implementation, the step of ablating is performed on only one papillary muscle PM. In some implementations, the step of ablating the tissue is repeated on multiple papillary muscles PM. In some implementations, after the tissue of a papillary muscle PM has been ablated to disrupt the conductive pathways 1600, the non-conductive ends 1612 no longer transmit a conductive signal to the papillary muscle PM so that the papillary muscle PM is relaxed during systole and does not contract at all or does not contract to the same degree. That is, the non-conductive ends 1612 of the conductive pathways 1600 are no longer able to trigger the papillary muscles PM so that the papillary muscles PM cannot be contracted to apply tension to the chordae tendineae CT, or does contract but not to the same degree as before the ablation procedure. Consequently, as is shown in FIG. 77, the leaflets 20, 22 of the mitral valve MV are able to coapt. Because the mitral valve can now close, the valve regurgitation present in FIG. 75 is no longer an issue even though the left ventricle LV remains unable to fully contract.

### Examples

Example 1. A device for repairing a native valve, comprising:

a first surface and a second surface, wherein the first surface and the second surface are movable between an open condition and a closed condition to pinch a first leaflet and a second leaflet of the native valve therebetween; and

wherein the device is configured to attach the first leaflet to the second leaflet.

Example 2.The device of example 1, wherein the device is configured to directly attach the first leaflet to the second leaflet.

Example 3. The device of example 1 or 2, wherein the device is configured to attach the first leaflet to the second leaflet by fusing the first leaflet to the second leaflet.

Example 4. The device of any of examples 1-3, wherein the first surface is a first contact surface on a first arm of the device, and the second surface is a second contact surface on a second arm of the device, wherein the first contact surface and the second contact surface are configured to output energy to the first leaflet and the second leaflet, respectively, to fuse the first leaflet to the second leaflet.

Example 5. The device of example 4, wherein the first contact surface and the second contact surface are configured to fuse the first leaflet to the second leaflet at two or more spaced apart locations.

Example 6. The device of example 5, wherein the first surface has an inner face that is U-shaped.

Example 7. The device of any of examples 1-6, further comprising a coaptation element, wherein the first surface is configured to capture the first leaflet against the coaptation element.

Example 8. The device of example 7, wherein the second surface is configured to capture the second leaflet against the coaptation element.

Example 9. The device of example 8, wherein the second surface is movable to capture the second leaflet independent of the first surface.

Example 10. The device of example 8 or 9, wherein the coaptation element is movable from between the first leaflet and the second leaflet prior to attaching the first leaflet to the second leaflet.

Example 11. The device of example 8 or 9, wherein the coaptation element is movable from between the first leaflet and the second leaflet after the first leaflet is attached to the second leaflet.

Example 12. The device of any of examples 7-11, wherein the first surface is configured to fuse the first leaflet to the second leaflet at a first location lateral to the coaptation element and at a second location lateral to the coaptation element and opposite the first location.

Example 13. The device of any of example 7, wherein the first surface is configured to capture the first leaflet against a first portion of the coaptation element and against a second portion of the coaptation element, and wherein the first surface is configured to fuse the first leaflet to the second leaflet at a location between the first portion and the second portion.

Example 14. The device of example 1, wherein the device is configured to attach the first leaflet to the second leaflet indirectly via a coaptation element.

Example 15. The device of example 14, wherein the first surface is configured to fuse the first leaflet to the coaptation element.

Example 16. The device of example 15, wherein the second surface is configured to fuse the second leaflet to the coaptation element.

Example 17. The device of example 16, wherein the first surface is configured to fuse the first leaflet to the coaptation element independent of the second surface.

Example 18. The device of any of examples 14-17, wherein the coaptation element has an exterior surface configured to be fused to the first leaflet, wherein the exterior surface is cylindrical.

Example 19. The device of any of examples 14-17, wherein the coaptation element has an exterior surface configured to be fused to the first leaflet, wherein the exterior surface is conical.

Example 20. The device of any of examples 18 or 19, wherein the exterior surface comprises collagen.

Example 21. The device of any of examples 1-20, further comprising a third surface and a fourth surface, wherein the third surface and the fourth surface are movable between an open condition and a closed condition to pinch the first leaflet and the second leaflet of the native valve therebetween at a second location spaced apart from the first location at which the first surface and the second surface pinch the first leaflet and the second leaflet together.

Example 22. The device of any of examples 1-21 wherein the first surface comprises a first inner face and the second surface comprises a second inner face, wherein the first inner face and the second inner face have complementary textured surface configurations.

Example 23. The device of example 22, wherein textured surface configuration comprises an undulating surface.

Example 24. The device of example 22, wherein textured surface configuration comprises a plurality of projections.

Example 25. The device of any of examples 3-24, further comprising a radio frequency generator operatively coupled to one or more of the first surface and the second surface.

Example 26. The device of example 1, wherein the device is configured to attach the first leaflet to the second leaflet with an adhesive.

Example 27. The device of example 26, further comprising a coaptation element, wherein the first surface is configured to capture the first leaflet against the coaptation element.

Example 28. The device of example 27, wherein the adhesive is embedded in the coaptation element.

Example 29. The device of example 28, wherein the coaptation element comprises a spongey material infused with the adhesive.

Example 30. The device of example 26, further comprising an adhesive delivery conduit configured to deliver the adhesive between the first leaflet and the second leaflet.

Example 31. The device of example 30, wherein the adhesive delivery conduit includes a static mixer configured to mix the adhesive prior to the adhesive being delivered between the first leaflet and the second leaflet.

Example 32. The device of example 26, wherein at least one component of the adhesive is encapsulated by an encapsulating structure.

Example 33. The device of example 32, wherein the encapsulating structure is configured to de-encapsulate when the adhesive is positioned between the first leaflet and the second leaflet, and the first surface and the second surface pinch the first leaflet and second leaflet therebetween.

Example 34. The device of example 32, wherein the encapsulating structure is dissolvable.

Example 35. The device of example 32, wherein the adhesive is a two-component adhesive.

Example 36. The device of example 27, wherein the adhesive is embedded in a substate attached to an exterior surface of the coaptation element.

Example 37. The device of example 27, wherein the adhesive is disposed onto a substrate attached to an exterior surface of the coaptation element.

Example 38. The device of example 27, wherein the adhesive is disposed on an exterior surface of the coaptation element.

Example 39. The device of example 38, wherein the exterior surface of the coaptation element includes a textured surface configuration.

Example 40. The device of example 39, wherein the textured surface configuration comprises a plurality of recesses and projections.

Example 41. A system for repairing a native valve, comprising:

a delivery system including a catheter; and

the device of any of examples 1-40 operatively coupled to a distal end of the catheter.

Example 42. A method for repairing a native valve, comprising:

pinching a first leaflet of the native valve and a second leaflet of the native valve together; and

attaching the first leaflet to the second leaflet.

Example 43. The method of example 42, wherein attaching the first leaflet to the second leaflet further comprises fusing the first leaflet to the second leaflet.

Example 44. The method of example 43, further comprising fusing the first leaflet to the second leaflet with radio frequency energy.

Example 45. The method of example 43 or 44 wherein fusing the first leaflet to the second leaflet further comprises fusing the first leaflet to the second leaflet at two or more spaced apart locations.

Example 46. The method of any of examples 42-45 further comprising capturing the first leaflet against a coaptation element.

Example 47. The method of example 46, further comprising capturing the second leaflet against the coaptation element.

Example 48. The method of example 47, wherein the second leaflet is captured independent of the first leaflet.

Example 49. The method of example 47, further comprising removing the coaptation element from between the first leaflet and the second leaflet prior to attaching the first leaflet to the second leaflet.

Example 50. The method of example 47, further comprising removing the coaptation element from between the first leaflet and the second leaflet after attaching the first leaflet to the second leaflet.

Example 51. The method of example 47, wherein fusing the first leaflet to the second leaflet further comprises fusing the first leaflet to the second leaflet at a first location lateral to the coaptation element and fusing the first leaflet to the second leaflet at a second location lateral to the coaptation element and opposite the first location.

Example 52. The method of any of example 47, wherein capturing the first leaflet against the coaptation element further comprises pinching the first leaflet against the coaptation element at a first location and at a second location spaced apart from the first location.

Example 53. The method of example 52, wherein fusing the first leaflet to the second leaflet further comprising fusing the first leaflet to the second leaflet at a third location between the first location and the second location.

Example 54. The method of example 47, wherein attaching the first leaflet to the second leaflet further comprises fusing the first leaflet and the second leaflet to an exterior surface of the coaptation element.

Example 55. The method of any of examples 42-54, wherein pinching the first leaflet of the native valve and the second leaflet of the native valve together further comprises indenting the first leaflet and the second leaflet.

Example 56. The method of any of examples 42-55, wherein pinching the first leaflet of the native valve and the second leaflet of the native valve together further comprises creating an undulating pattern in the first leaflet and the second leaflet.

Example 57. The method of example 42, wherein attaching the first leaflet to the second leaflet further comprises attaching the first leaflet to the second leaflet with an adhesive.

Example 58. The method of example 57, further comprising capturing the first leaflet against a coaptation element.

Example 59. The method of example 58, wherein pinching the first leaflet of the native valve and the second leaflet of the native valve together, further comprises pinching the coaptation element between the first leaflet and the second leaflet.

Example 60. The method of example 59, wherein the adhesive is embedded in the coaptation element and pinching the coaptation element between the first leaflet and the second leaflet releases the adhesive.

Example 61. The method of example 57, delivering the adhesive between the first leaflet and the second leaflet via an adhesive delivery conduit.

Example 62. The method of example 61, further comprising mixing the adhesive within the adhesive delivery conduit.

Example 63. The method of example 57, wherein the adhesive is encapsulated by an encapsulating structure.

Example 64. The method of example 63, further comprises positioning the adhesive encapsulated in the encapsulating structure between the first leaflet and the second leaflet, and wherein pinching the first leaflet of the native valve and the second leaflet of the native valve together further comprises de-encapsulating the adhesive.

Example 65. The method of example 64, wherein the de-encapsulating the adhesive further comprises bursting the encapsulating structure.

Example 66. The method of example 58, further comprising disposing the adhesive on a substrate attached to an exterior surface of the coaptation element.

Example 67. The method of example 58, further comprising disposing the adhesive onto an exterior surface of the coaptation element.

Example 68. The method of example 67, wherein the exterior surface of the coaptation element includes a plurality of recesses and projections, and wherein the adhesive is received in the plurality of recesses.

Example 69. A method for repairing a native valve, comprising:

inserting a catheter through the native valve;

extending an ablation device from a distal end of the catheter to a papillary muscle; and

ablating tissue of the papillary muscle with the ablation device to disable a conductive pathway to the papillary muscle.

Example 70. The method of example 69, wherein the native valve is a mitral valve, and the catheter is inserted through the mitral valve.

Example 71. The method of example 69, wherein the native valve is a tricuspid valve, and the catheter is inserted through the tricuspid valve.

Example 72. The method of example 69, further comprising a step of detecting electrical signals transmitted through the conductive pathway.

Example 73. The method of example 72, further comprising a step of selecting the papillary muscle to ablate based on detected the electrical signals.

Example 74. The method of example 69, further comprising a step of selecting the papillary muscle to ablate based on a geometry of a leaflet of the native valve or a geometry of a ventricle connected to the native valve.

Example 75. The method of example 69, wherein the step of ablating is performed only on one papillary muscle.

Example 76. The method of example 69, wherein the step of ablating is repeated for each papillary muscle.

Example 77. The method of example 69, wherein the step of ablating is repeated for a single papillary muscle.

Example 78. The method of example 69, wherein the ablation device is a radiofrequency ablation device.

Example 79. The method of example 69, wherein the ablation device is a cryoablation device.

Example 80. The method, device, or system of any preceding example, wherein one or more components used in the method or included in the device or system are sterilized.

Any of the various systems, assemblies, devices, components, apparatuses, etc. in this disclosure can be sterilized (e.g., with heat, radiation, ethylene oxide, hydrogen peroxide, etc.) to ensure they are safe for use with patients, and the methods herein can comprise (or additional methods comprise or consist of) sterilization of the associated system, device, component, apparatus, etc. (e.g., with heat, radiation, ethylene oxide, hydrogen peroxide, etc.).

While various inventive aspects, concepts and features of the disclosures can be described and illustrated herein as embodied in combination in the examples herein, these various aspects, concepts, and features can be used in many alternative examples, either individually or in various combinations and sub-combinations thereof. Unless expressly excluded herein all such combinations and sub-combinations are intended to be within the scope of the present application. Still further, while various alternative examples as to the various aspects, concepts, and features of the disclosures—such as alternative materials, structures, configurations, methods, devices, and components, alternatives as to form, fit, and function, and so on—may be described herein, such descriptions are not intended to be a complete or exhaustive list of available alternative examples, whether presently known or later developed. Those skilled in the art can readily adopt one or more of the inventive aspects, concepts, or features into additional examples and uses within the scope of the present application even if such examples are not expressly disclosed herein.

Additionally, even though some features, concepts, or aspects of the disclosures may be described herein as being a preferred arrangement or method, such description is not intended to suggest that such feature is required or necessary unless expressly so stated. Still further, example or representative values and ranges may be included to assist in understanding the present application, however, such values and ranges are not to be construed in a limiting sense and are intended to be critical values or ranges only if so expressly stated.

Moreover, while various aspects, features and concepts may be expressly identified herein as being inventive or forming part of a disclosure, such identification is not intended to be exclusive, but rather there may be inventive aspects, concepts, and features that are fully described herein without being expressly identified as such or as part of a specific disclosure, the disclosures instead being set forth in the appended claims. Descriptions of example methods or processes are not limited to inclusion of all steps as being required in all cases, nor is the order that the steps are presented to be construed as required or necessary unless expressly so stated. The words used in the claims have their full ordinary meanings and are not limited in any way by the description of the examples in the specification.

What is claimed is:

1. A method for repairing a native valve, comprising:
advancing a valve repair device to the native valve with a catheter;
pinching a first leaflet of the native valve and a second leaflet of the native valve together with the valve repair device;
fusing the first leaflet to the second leaflet by applying radiofrequency energy to the first leaflet and the second leaflet with the valve repair device; and
removing the valve repair device from the native valve with the catheter.

2. The method of claim 1, wherein the valve repair device pinches the first leaflet and the second leaflet at two spaced apart locations and fuses the first leaflet to the second leaflet at the two spaced apart locations.

3. The method of claim 1, further comprising capturing the first leaflet against a coaptation element of the valve repair device.

4. The method of claim 3, further comprising capturing the second leaflet against the coaptation element of the valve repair device.

5. The method of claim 4, wherein the second leaflet is captured independent of the first leaflet.

6. The method of claim 4, further comprising removing the coaptation element from between the first leaflet and the second leaflet prior to attaching the first leaflet to the second leaflet.

7. The method of claim 4, further comprising removing the coaptation element from between the first leaflet and the second leaflet after attaching the first leaflet to the second leaflet.

8. The method of claim 4, wherein fusing the first leaflet to the second leaflet further comprises fusing the first leaflet to the second leaflet at a first location lateral to the coaptation element and fusing the first leaflet to the second leaflet at a second location lateral to the coaptation element and opposite the first location.

9. The method of claim 4, wherein capturing the first leaflet against the coaptation element further comprises pinching the first leaflet against the coaptation element at a first location and at a second location spaced apart from the first location.

10. A method for repairing a native valve, comprising:
advancing a valve repair device to the native valve with a catheter;
pinching a first leaflet of the native valve and a second leaflet of the native valve together with the valve repair device;
delivering an adhesive between the first leaflet and the second leaflet to adhere the first leaflet and the second leaflet together; and
removing the valve repair device from the native valve with the catheter.

11. The method of claim 10, wherein the adhesive is delivered through the catheter.

12. The method of claim 10, wherein the adhesive is delivered with the valve repair device.

13. The method of claim 10, further comprising capturing the first leaflet against a coaptation element.

14. The method of claim 13, wherein pinching the first leaflet of the native valve and the second leaflet of the native valve together, further comprises pinching the coaptation element between the first leaflet and the second leaflet.

15. The method of claim 14, wherein the adhesive is embedded in the coaptation element and pinching the coaptation element between the first leaflet and the second leaflet releases the adhesive.

16. The method of claim 10, wherein the adhesive is delivered between the first leaflet and the second leaflet via an adhesive delivery conduit.

17. The method of claim 10, wherein the adhesive is encapsulated by an encapsulating structure.

18. The method of claim 17, further comprises positioning the adhesive encapsulated in the encapsulating structure between the first leaflet and the second leaflet, and wherein pinching the first leaflet of the native valve and the second leaflet of the native valve together de-encapsulates the adhesive.

19. A method for repairing a native valve, comprising:
inserting a catheter through the native valve;
extending an ablation device from a distal end of the catheter to a papillary muscle; and
ablating tissue of the papillary muscle with the ablation device to disable a conductive pathway to the papillary muscle.

20. The method of claim 19, further comprising removing the ablation device.
